(19) United States
(12) Patent Application Publication
(10) Pub. No.: US 2005/0124625 A1
(43) Pub. Date: Jun. 9, 2005

(54) Piperazine derivatives and their use as modulators of nuclear hormone receptor function

(76) Inventors: Mark E. Salvati, Lawrenceville, NJ (US); Soong-Hoon Kim, Titusville, NJ (US)

Correspondence Address: STEPHEN B. DAVIS;BRISTOL-MYERS SQUIBB COMPANY, PATENT DEPARTMENT, P O BOX 4000, PRINCETON, NJ, 08543-4000, US

(21) Appl. No.: 10/969,594

(22) Filed: Oct. 20, 2004

Related U.S. Application Data
(60) Provisional application No. 60/513,175, filed on Oct. 21, 2003.

(51) Int. Cl. A61K 31/53; A61K 31/506; A61K 31/496; C07D 43/14

(52) U.S. Cl. 514/253.01; 514/253.04; 514/242; 514/253.06; 514/252.18; 544/360; 544/362; 544/363; 544/295; 544/182

(57) ABSTRACT

The present invention provides piperazine derivatives and methods of using such compounds in the treatment of nuclear hormone receptor-associated conditions such as cancer and immune disorders.

### CROSS-REFERENCE TO RELATED APPLICATIONS

The present application claims the priority benefit of U.S. Provisional Application No. 60/513,175, filed Oct. 21, 2003.

### FIELD OF THE INVENTION

The present invention relates to piperazine derivatives, to methods of using such compounds in the treatment of nuclear hormone receptor-associated conditions such as cancer, and to pharmaceutical compositions containing such compounds.

### BACKGROUND OF THE INVENTION

Nuclear hormone receptors (NHR's) constitute a large super-family of ligand-dependent and sequence-specific transcription factors. Members of this family influence transcription either directly through specific binding to the promoter target genes (Evans, in Science 240: 889-895 (1988)), or indirectly via protein-protein interactions with other transcription factors (Jonat et al., Cell 62: 1189-1204 (1990), Schuele et al., Cell 62: 1217-1226 (1990), and Yang-Yen et al., Cell 62: 1205-1215 (1990)). The nuclear hormone receptor super-family (also known in the art as the “steroid/thyroid hormone receptor super-family”) includes receptors for a variety of hydrophobic ligands, including cortisol, aldosterone, estrogen, progesterone, testosterone, vitamin D3, thyroid hormone and retinoic acid (Evans, 1988, supra). In addition to these conventional NHR'S, the super-family contains a number of proteins that have no known ligands, termed orphan nuclear hormone receptors (Mangelsdorf et al., Cell 83: 835-839 (1995), O'Malley et al., Mol. Endocrinol. 10: 1293 (1996), Enmark et al., Mol. Endocrinol. 10, 1293-1307 (1996) and Giguere, Endocrin. Rev. 20, 689-725 (1999)).

The conventional NHR's are generally transactivators in the presence of ligand, and can either be active repressors or transcriptionally inert in the absence of ligand. Some orphan receptors behave as if they are transcriptionally inert in the absence of ligand. Others, however, behave as either constitutive activators or repressors. These orphan NHR's are either under the control of ubiquitous ligands that have not been identified, or do not need to bind ligand to exert these activities.

In common with other transcription factors, the NHR's have a modular structure, being comprised of three distinct domains: an N-terminal domain of variable size containing a transcriptional activation function AF-1, a highly-conserved DNA binding domain, and a moderately conserved ligand-binding domain. The ligand-binding domain is not only responsible for binding the specific ligand but also contains a transcriptional activation function called AF-2 and a dimerisation domain (Wurtz et al., Nature Struc. Biol. 3, 87-94 (1996), Parker et al., Nature Struc. Biol. 3, 113-115 (1996) and Kumar et al., Steroids 64, 310-319 (1999)). Although the overall protein sequence of these receptors can vary significantly, all share both a common structural arrangement indicative of divergence from an ancestral archetype and substantial homology (especially, sequence identity) at the ligand-binding domain.

A sub-family of NHR's are the steroid binding NHR's (SB-NHR's). The SB-NHR's are related in that they share a stronger sequence homology to one another, particularly in the ligand-binding domain (LBD), than to other members of the NHR super-family (Evans, 1988, supra) and they all utilize steroid-based ligands. Some examples of this sub-family of NHR's are the androgen receptor (AR), the estrogen receptor (ER), the progesterone receptor (PR), the glucocorticoid receptor (GR), the mineralocorticoid receptor (MR), the aldosterone receptor (ALDR), and the steroid and xenobiotic receptor (SXR) (Evans et al., WO 99/35246). Based on the strong sequence homology in the LBD, several orphan receptors may also be members of the SB-NHR sub-family.

Consistent with the high sequence homology found in the LBD for each of the SB-NHR's, the natural ligands for each is derived from a common steroid core. Examples of some steroid-based ligands utilized by members of the SB-NHR's include cortisol, aldosterone, estrogen, progesterone, testosterone, and dihydrotestosterone. Specificity of a particular steroid-based ligand for one SB-NHR versus another SB-NHR is obtained by differential substitution about the steroid core. High affinity binding to a particular SB-NHR, coupled with high level specificity for that particular SB-NHR, can be achieved with minor structural changes about the steroid core (e.g., Waller et al., Toxicol. Appl. Pharmacol. 137, 219-227 (1996) and Mekenyan et al., Environ. Sci. Technol. 31, 3702-3711 (1997), binding affinity for progesterone towards the androgen receptor as compared to testosterone).

Numerous synthetically derived steroidal and non-steroidal agonists and antagonists have been described for the members of the SB-NHR family. Many of these agonist and antagonist ligands are used clinically in man to treat a variety of medical conditions. RU486 is an example of a synthetic agonist of PR, which is utilized as a birth control agent (Vegeto et al., Cell 69: 703-713 (1992)), and Flutamide is an example of an antagonist of AR, which is utilized for the treatment of prostate cancer (Neri et al, Endo. 91, 427-437 (1972)). Tamoxifen is a tissue specific modulator of ER function used in the treatment of breast cancer (Smigel, J. Natl. Cancer Inst. 90, 647-648 (1998)). Tamoxifen can function as an antagonist of ER in breast tissue while acting as an agonist of ER in bone (Grese et al., Proc. Natl. Acad. Sci. USA 94, 14105-14110 (1997)). Because of the tissue selective effects seen for Tamoxifen, this agent and agents like it are referred to as “partial-agonist” or “partial-antagonist”.

Besides synthetically derived non-endogenous ligands, non-endogenous ligands for NHR's can be obtained from food sources (Regal et al., Proc. Soc. Exp. Biol. Med. 223, 372-378 (2000) and Hempstock et al., J. Med. Food 2, 267-269 (1999)). The flavanoid phytoestrogens are an example of an unnatural ligand for SB-NHR's that are readily obtained from a food source, e.g., soy (Quella et al., J. Clin. Oncol. 18, 1068-1074 (2000) and Banz et al., J. Med. Food 2, 271-273 (1999)). Because the transcriptional activity of individual NHR's can be modulated by the addition of a small molecule ligands, the NHR's are good targets for the development of pharmaceutical agents for a variety of disease states.

As mentioned above, non-natural ligands can be synthetically engineered to serve as modulators of the function of NHR's. In the case of SB-NHR's, engineering of an unnatural ligand can include the identification of a core structure which mimics the natural steroid core system. This can be achieved by random screening against several SB-NHR's or through directed approaches using the available crystal structures of a variety of NHR ligand-binding domains (Bourguet et al., Nature 375, 377-382 (1995), Brzozowski, et al., Nature 389, 753-758 (1997), Shiau et al., Cell 95, 927-937 (1998) and Tanenbaum et al., Proc. Natl. Acad. Sci. USA 95, 5998-6003 (1998)). Differential substitution about such a steroid mimic core can provide agents with selectivity for one receptor versus another. In addition, such modifications can be employed to obtain agents with agonist or antagonist activity for a particular SB-NHR. Differential substitution about the steroid mimic core can result in the formation of a series of high affinity agonists and antagonists with specificity for, for example, ER versus PR versus AR versus GR versus MR. Such an approach of differential substitution has been reported, for example, for quinoline-based modulators of steroid NHR in J. Med. Chem., 41, 623 (1999); WO 97/49709; U.S. Pat. No. 5,696,133; U.S. Pat. No. 5,696,130; U.S. Pat. No. 5,696,127; U.S. Pat. No. 5,693,647; U.S. Pat. No. 5,693,646; U.S. Pat. No. 5,688,810; U.S. Pat. No. 5,688,808 and WO 9619458, all incorporated herein by reference. Compounds that reportedly modulate AR are also disclosed in EP 1 222 242 A1 to Yamanouchi Pharmaceuticals (WO 0017163).

There remains an unfulfilled need for compounds that may be used as agonists and/or antagonists of SB-NHR's. The present invention is directed to these, as well as other important ends.

### SUMMARY OF THE INVENTION

The present invention provides methods of treating NHR-associated conditions with piperazine compounds and salts and pharmaceutical compositions thereof, which compounds are especially useful as modulators of nuclear hormone receptor function. Specifically, in one aspect of the invention, there is provided a method of treating a NHR-associated condition comprising administering to a subject in need of treatment thereof, an effective amount of a compound of Formula (I): 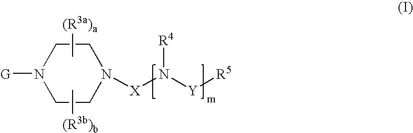
wherein:
G is Ar¹, Ar², or Ar³;
Ar¹ is a bicyclic or tricyclic aryl or heteroaryl optionally substituted with one to four R⁶;
Ar² is a monocyclic five-membered heteroaryl optionally substituted with one to two R⁶; 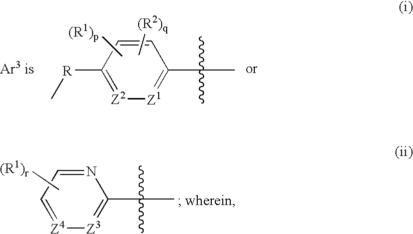
Z¹ and Z² are each independently nitrogen or carbon, the carbon atoms of Z¹ and Z² each being bonded to a hydrogen atom or being substituted with a group R¹ or R²;
one of Z³ and Z⁴ is nitrogen, and the other of Z³ and Z⁴ is carbon, the carbon atom of Z³ or Z⁴ being bonded to a hydrogen atom or being substituted with a group R¹;
R is R², hydrogen, halogen, haloalkyl, haloalkoxy, alkyl, substituted alkyl, —C(═O)R⁷, —C(═O)—O—R⁷, or —C(═O)NR⁹R¹⁰, and additionally, when either (i) p and q taken together are at least two, and/or (ii) q is at least one, then R may also be cyano or nitro, provided that when Z¹ and Z² are carbon and R is cyano, then the carbon atom of Z¹ is substituted with the group R¹ or R²;
R¹ is alkyl, substituted alkyl, alkenyl, substituted alkenyl, alkynyl, substituted alkynyl, halo, cyano, nitro, cycloalkyl, substituted cycloalkyl, aryl, substituted aryl, heterocyclo, substituted heterocyclo, —OC(═O)R⁷, —C(═O)—O—R⁷, —C(═O)R⁷, —C(═S)R⁷, —C(═O)NR⁹R¹⁰, —CR¹¹R¹²OR⁷, —OR⁷, —NR⁹R¹⁰, —SR⁷, —S(═O)R⁷, —SO₂R⁷, —SO₂OR⁷, and/or —SO₂NR⁷R⁸;
R² is optionally-substituted aryl, cycloalkyl, and/or heterocyclo;
R^{3a} and R^{3b} are each independently alkyl, substituted alkyl, alkenyl, substituted alkenyl, alkynyl, substituted alkynyl, cycloalkyl, substituted cycloalkyl, heterocyclo, substituted heterocyclo, aryl, substituted aryl, cyano, —OR¹³, —C(═O)R¹³, —OC(═O)R¹³, —C(═O)OR¹³, —C(═O)NR¹⁴R¹⁵, —SO₂R¹³, —SO₂OR¹³, —SO₂NR¹⁴R¹⁵, and/or a carbamoyl group which may be substituted by 1 or 2 lower alkyl;
X is —C(═O)—, —C(═S)—, or —SO₂—;
R⁴ is hydrogen, alkyl, substituted alkyl, cyano, -O-lower alkyl, a carbamoyl group which may be substituted by 1 or 2 lower alkyl; a lower alkylene-C(═O)—, or a lower alkylene-O—C(═O)— group;
Y is a bond, lower alkylene, —C(═O)—, or —SO₂—;
R⁵ is alkyl, substituted alkyl, alkenyl, substituted alkenyl, cycloalkyl, substituted cycloalkyl, heterocyclo, substituted heterocyclo, aryl, substituted aryl, —OR⁷, —C(═O)R⁷, —OC(═O)R⁷, —C(═O)OR⁷, or amido which may be substituted by 1 or 2 lower alkyl, aryl, heterocycle or cycloalkyl each of which in turn may optionally be substituted;
alternatively, when m is 1, R⁴ and R⁵ may be linked together to optionally form a five- or six-membered heterocycle;
R⁶ is alkyl, substituted alkyl, alkenyl, substituted alkenyl, alkynyl, substituted alkynyl, halo, cycloalkyl, substituted cycloalkyl, aryl, substituted aryl, heterocyclo, substituted heterocyclo, cyano, nitro, —OC(═O)R⁷, —C(═O)—O—R⁷, —C(═O)R⁷, —C(═S)R⁷, —C(═O)NR⁹R¹⁰, —CR¹¹R¹²OR⁷, —OR⁷, —NR⁹R¹⁰, —SR⁷, —S(═O)R⁷, —SO₂R⁷, —SO₂OR⁷, —SO₂NR⁷R⁸, —P(═O)(OR⁷)(OR⁸), —P(═O)(R⁷)(R⁸), and/or —P(═O)(R⁸)(NHR⁹);
R⁷, R⁸ and R¹³ are each, independently of each other, and at each occurrence, independently hydrogen, alkyl, substituted alkyl, alkenyl, substituted alkenyl, alkynyl, substituted alkynyl, cycloalkyl, substituted cycloalkyl, heterocyclo, substituted heterocyclo, aryl, and/or substituted aryl, except when R⁷ or R¹³ is attached to a sulfonyl group as in —S(═O)R⁷, S(═O)R¹³, —SO₂R⁷, —SO₂R¹³, —SO₂OR⁷, and —SO₂OR¹³, then R⁷ and R¹³ are not hydrogen;
R⁹ and R¹⁰ are each independently hydrogen, alkyl, substituted alkyl, alkenyl, substituted alkenyl, alkynyl, substituted alkynyl, cycloalkyl, substituted cycloalkyl, heterocyclo, substituted heterocyclo, aryl, substituted aryl, —C(═O)R⁷, —C(═O)NHR⁷, —SO₂OR⁷, and/or —SO₂NR⁷R⁸;
R¹¹ and R¹² are each independently hydrogen, alkyl, substituted alkyl, alkenyl, substituted alkenyl, alkynyl, substituted alkynyl, cycloalkyl, substituted cycloalkyl, heterocyclo, substituted heterocyclo, aryl, substituted aryl, halo, cyano, hydroxylamine, hydroxamide, alkoxy, substituted alkoxy, —NR⁷R⁸, thiol, alkylthio, and/or substituted alkylthio;
R¹⁴ and R¹⁵ are each independently hydrogen, alkyl, substituted alkyl, cycloalkyl, substituted cycloalkyl, heterocyclo, substituted heterocyclo, aryl, and/or substituted aryl;
one of a and b is 1, and the other of a and b is 0 or 1;
m is 0 or 1;
p and q are 0, 1 and/or 2; and
r is 0, 1, 2 or 3;
or a pharmaceutically acceptable salt, solvate or N-oxide thereof.

According to another aspect of the invention, there are provided compounds useful in treating NHR-associated conditions, having the formula (I), 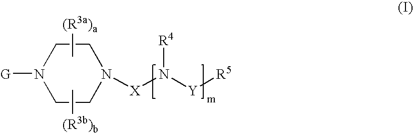
wherein:
G is Ar^{1a}, Ar^{2a}, or Ar^{3a};
Ar^{1a} is a bicyclic or tricyclic aryl or heteroaryl optionally substituted with one to four
R⁶, provided, however, that if Ar¹ is 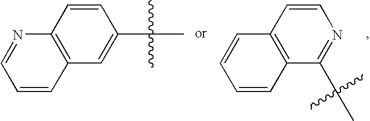
   then a and b taken together are at least two;
Ar^{2a} is a monocyclic five-membered heteroaryl optionally substituted with one to two R⁶; 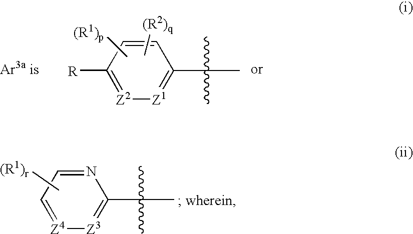
Z¹ and Z² are each independently nitrogen or carbon, the carbon atoms of Z¹ and Z² each being bonded to a hydrogen atom or being substituted with a group R¹ or R²;
one of Z³ and Z⁴ is nitrogen and the other of Z³ and Z⁴ is carbon, the carbon atom of Z³ or Z⁴ being bonded to a hydrogen atom or being substituted with a group R¹; provided however, that if Z¹ or Z⁴ is nitrogen, then a and b taken together are at least two;
R is R², hydrogen, halogen, haloalkyl, haloalkoxy, alkyl, substituted alkyl, —C(═O)R⁷, —C(═O)—O—R⁷, or —C(═O)NR⁹R¹⁰, and additionally, when either (i) p and q taken together are at least two, and/or (ii) q is at least one, then R may also be cyano or nitro, provided that if Z¹ and Z² are carbon and R is cyano, then the carbon atom of Z¹ is substituted with the group R¹ or R²;
R¹ is alkyl, substituted alkyl, alkenyl, substituted alkenyl, alkynyl, substituted alkynyl, halo, cyano, nitro, cycloalkyl, substituted cycloalkyl, aryl, substituted aryl, heterocyclo, substituted heterocyclo, —OC(═O)R⁷, —C(═O)—O—R⁷, —C(═O)R⁷, —C(═S)R⁷, —C(═O)NR⁹R¹⁰, —CR¹¹R¹²OR⁷, —OR⁷, —NR⁹R¹⁰, —SR⁷, —S(═O)R⁷, —SO₂R⁷, —SO₂OR⁷, and/or —SO₂NR⁷R⁸;
R² is optionally-substituted aryl, cycloalkyl, or heterocyclo;
R^{3a} and R^{3b} are each independently alkyl, substituted alkyl, alkenyl, substituted alkenyl, alkynyl, substituted alkynyl, cycloalkyl, substituted cycloalkyl, heterocyclo, substituted heterocyclo, aryl, substituted aryl, cyano, —OR¹³, —C(═O)R¹³, —OC(═O)R¹³, —C(═O)OR¹³, —C(═O)NR¹⁴R¹⁵, —SO₂R¹³, —SO₂OR¹³, —SO₂NR¹⁴R¹⁵, and/or a carbamoyl group which may be substituted by 1 or 2 lower alkyl;
X is —C(═O)—, —C(═S)—, or —SO₂—;
R⁴ is hydrogen, alkyl, substituted alkyl, cyano, —O-lower alkyl, a carbamoyl group which may be substituted by 1 or 2 lower alkyl; a lower alkylene-C(═O)—, or a lower alkylene-O—C(═O)— group;
Y is a bond, lower alkylene, —C(═O)—, or —SO₂—;
R⁵ is alkyl, substituted alkyl, alkenyl, substituted alkenyl, cycloalkyl, substituted cycloalkyl, heterocyclo, substituted heterocyclo, aryl, substituted aryl, —OR⁷, —C(═O)R⁷, —OC(═O)R⁷, —C(═O)OR⁷, or amido which may be substituted by 1 or 2 lower alkyl, aryl, heterocycle or cycloalkyl each of which in turn may optionally be substituted;
alternatively, when m is 1, R⁴ and R⁵ may be linked together to optionally form a five- or six-membered heterocycle;
R⁶ is alkyl, substituted alkyl, alkenyl, substituted alkenyl, alkynyl, substituted alkynyl, halo, cycloalkyl, substituted cycloalkyl, aryl, substituted aryl, heterocyclo, substituted heterocyclo, cyano, nitro, —OC(═O)R⁷, —C(═O)—O—R⁷, —C(═O)R⁷, —C(═S)R⁷, —C(═O)NR⁹R¹⁰, —CR¹¹R¹²OH, —CH₂OR⁷, —OR⁷, —NR⁹R¹⁰, —SR⁷, —S(═O)R⁷, —SO₂R⁷, —SO₂NR⁷R⁸, —P(═O)(OR⁷)(OR⁸), —P(═O)(R⁷)(R⁸), and/or —P(═O)(R⁸)(NHR⁹);
R⁷, R⁸ and R¹³ are each independently hydrogen, alkyl, substituted alkyl, alkenyl, substituted alkenyl, alkynyl, substituted alkynyl, cycloalkyl, substituted cycloalkyl, heterocyclo, substituted heterocyclo, aryl, and/or substituted aryl, except when R⁷ or R¹³is attached to a sulfonyl group as in —S(═O)R⁷, —S(═O)R¹³, —SO₂R⁷, —SO₂R¹³, —SO₂OR⁷, and —SO₂OR¹³, then R⁷ and R¹³ are not hydrogen;
R⁹ and R¹⁰ are each independently hydrogen, alkyl, substituted alkyl, alkenyl, substituted alkenyl, alkynyl, substituted alkynyl, cycloalkyl, substituted cycloalkyl, heterocyclo, substituted heterocyclo, aryl, substituted aryl, —C(═O)R⁷, —C(═O)NHR⁷, —SO₂OR⁷, and/or —SO₂NR⁷R⁸;
R¹¹ and R¹² are each independently hydrogen, alkyl, substituted alkyl, alkenyl, substituted alkenyl, alkynyl, substituted alkynyl, cycloalkyl, substituted cycloalkyl, heterocyclo, substituted heterocyclo, aryl, substituted aryl, halo, cyano, hydroxylamine, hydroxamide, alkoxy, substituted alkoxy, —NR⁷R⁸, thiol, alkylthio, and/or substituted alkylthio;
R¹⁴ and R¹⁵ are each independently hydrogen, alkyl, substituted alkyl, cycloalkyl, substituted cycloalkyl, heterocyclo, substituted heterocyclo, aryl, and/or substituted aryl;
one of a and b is 1, and the other of a and b is 0 or 1;
m is 0 or 1;
p and q are 0, 1, and/or 2; and
r is 0, 1, 2, or 3;
or a pharmaceutically acceptable salt, solvate, or N-oxide thereof.

These as well as other important aspects of the invention will become more apparent from the following detailed description.

### FURTHER DESCRIPTION OF THE INVENTION

The following are definitions of terms used in the present specification. The initial definition provided for a group or term herein applies to that group or term throughout the present specification individually or as part of another group, unless otherwise indicated.

The terms “alkyl” and “alk” refers to a straight or branched chain alkane (hydrocarbon) radical containing from 1 to 12 carbon atoms, preferably 1 to 6 carbon atoms. Exemplary groups include, but are not limited to, methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl, dodecyl, and the like.

“Substituted alkyl” refers to an alkyl group substituted with one or more substituents, preferably 1 to 4 substituents, at any available point of attachment on the alkyl straight or branched chain. Exemplary substituents include one or more of the following groups: halo (e.g., a single halo substituent or multiple halo substituents forming, in the latter case, groups such as a perfluoroalkyl group or an alkyl group bearing Cl₃ or CF₃), nitro, cyano, hydroxy, alkoxy, haloalkoxy (e.g., trifluoromethoxy), —O-aryl, —O-heterocyclo, —O-alkylene-aryl, —O-haloalkyl, alkylthio, carboxy (i.e., —COOH), alkoxycarbonyl, alkylcarbonyloxy, carbamoyl, substituted carbamoyl, carbamate, substituted carbamate, urea, substituted urea, amidinyl, substituted amidinyl, aryl, heterocycle, cycloalkyl, —NR^{c}R^{d}, —OC(═O)NR^{c}R^{d}, —C(═O)NR^{c}R^{d}, —NR^{e}C(═O)NR^{c}R^{d}, —NR^{e}C(O)²—NR^{c}R^{d}—N(R^{e})S(O)₂NR^{c}R^{d}, —N(R^{e})P(O)₂NR^{c}R^{d}, (wherein each of R^{c} and R^{d} is independently selected from hydrogen, alkyl, aryl, and heterocyclo, and R^{e} is hydrogen, alkyl, or phenyl); and —SR^{f}, —S(═O)R^{g}, —S(O)₂R^{g}, —NR^{e}S(O)₂—R^{g}, —P(O)₂—R^{g}, —NR^{e}P(O)₂—R^{g}, —NR^{e}C(═O)R^{f}, —NR^{e}C(O)₂R^{f}, —OC(═O)R^{f}, —OC(═O)OR^{f}, —C(═O)OR^{f} or —C(═O)R^{f} (wherein R^{e} is defined as immediately above, R^{f} is hydrogen, alkyl, aryl or heterocyclo, and R^{g} is alkyl, aryl, or heterocyclo). In the aforementioned substituents, in each instance, the alkyl, aryl, heterocyclo or cycloalkyl groups (R^{c}, R^{d}, R^{f}, and R^{g}) in turn can be optionally substituted with one to four, preferably one to three further groups, selected from R^{k}, —O—R^{k}, cyano, nitro, haloalkyl, haloalkoxy, halogen, —NR^{k}R^{m}, —OC(═O)NR^{k}R^{m}, —C(═O)NR^{k}R^{m}, —NR^{k}C(═O)R^{m}, —SR^{k}, —S(═O)Rⁿ, —S(O)₂Rⁿ, —OC(═O)R^{k}, —C(═O)OR^{k}, —C(═O)R^{k}, phenyl, benzyl, phenyloxy, or benzyloxy, or a lower alkyl substituted with one to two of —O—R^{k}, cyano, nitro, haloalkyl, haloalkoxy, halogen, —NR^{k}R^{m}, —OC(═O)NR^{k}R^{m}, —C(═O)NR^{k}R^{m}, —NR^{k}C(═O)R^{m}, —SR^{k}, —S(═O)Rⁿ, —S(O)₂Rⁿ, —OC(═O)R^{k}, —C(═O)OR^{k}, —C(═O)R^{k}, phenyl, benzyl, phenyloxy, or benzyloxy, wherein R^{k} and R^{m} are selected from hydrogen, lower alkyl, hydroxy(lower alkyl), halo(lower alkyl), cyano(lower alkyl), and amino(lower alkyl), and Rⁿ is lower alkyl.

When a subscript is used following a group, as in C₁₋₄alkyl, this refers to the number of carbon atoms that the group may contain, in addition to heteroatoms or other substituents. Thus, for example, C_{1,4}alkyl refers to alkyl groups having from one to four carbon atoms; —O—C₁₋₃alkyl (or —O—C₁₋₃alkoxy) refers to alkoxy groups having from one to three carbon atoms, i.e., methoxy, ethoxy and propoxy; and optionally-substituted C₁₋₄alkyl refers to alkyl groups of one to four carbon atoms optionally substituted with one to four groups selected from those recited above for substituted alkyl.

As used herein, “alkylene” refers to a bivalent alkyl radical having the general formula —(CH₂)ₙ—, where n is 1 to 10. Non-limiting examples include methylene, dimethylene, trimethylene, tetramethylene, pentamethylene, and hexamethylene. The term “lower alkylene” herein refers to those alkylene groups having from about 1 to about 6 carbon atoms. “Substituted alkylene” refers to an alkylene group substituted with one or more substituents, preferably 1 to 4 substituents, at any available point of attachment. Exemplary substituents include, but are not limited to alkyl, substituted alkyl, and those groups recited above as exemplary alkyl substituents.

When the term alkyl is used as a subscript following another particularly-named group, as in “arylalkyl”, “substituted arylalkyl,” “cycloalkylalkyl,” etc., or as in hydroxy(lower alkyl), this refers to an alkyl group having one or two (preferably one) substituent selected from the other, particularly-named group. Thus, for example, arylalkyl includes benzyl, biphenyl and phenylethyl. A “substituted arylalkyl” will be substituted on the alkyl portion of the radical with one or more groups selected from those recited above for alkyl, and/or will be substituted on the aryl portion of the radical with one or more groups selected from those recited below for substituted aryl.

The term “alkenyl” refers to a straight or branched chain hydrocarbon radical containing from 2 to 12 carbon atoms and at least one carbon-carbon double bond. Exemplary groups include ethenyl or allyl. “Substituted alkenyl” refers to an alkenyl group substituted with one or more substituents, preferably 1 to 4 substituents, at any available point of attachment. Exemplary substituents include, but are not limited to, alkyl, substituted alkyl, and those groups recited above as exemplary alkyl substituents.

The term “alkenylene” refers to a straight or branched chain bivalent hydrocarbon radical containing from 2 to 12 carbon atoms and at least one carbon-carbon double bond. Exemplary groups include ethenylene or allylene. “Substituted alkenylene” refers to an alkenylene group substituted with one or more substituents, preferably 1 to 4 substituents, at any available point of attachment. Exemplary substituents include, but are not limited to, alkyl, substituted alkyl, and those groups recited above as exemplary alkyl substituents.

The term “alkynyl” refers to a straight or branched chain hydrocarbon radical containing from 2 to 12 carbon atoms and at least one carbon to carbon triple bond. Exemplary groups include ethynyl. “Substituted alkynyl” refers to an alkynyl group substituted with one or more substituents, preferably 1 to 4 substituents, at any available point of attachment. Exemplary substituents include, but are not limited to, alkyl, substituted alkyl, and those groups recited above as exemplary alkyl substituents.

The term “alkynylene” refers to a straight or branched chain bivalent hydrocarbon radical containing from 2 to 12 carbon atoms and at least one carbon to carbon triple bond. Exemplary groups include ethynylene. “Substituted alkynylene” refers to an alkynylene group substituted with one or more substituents, preferably 1 to 4 substituents, at any available point of attachment. Exemplary substituents include, but are not limited to, alkyl, substituted alkyl, and those groups recited above as exemplary alkyl substituents.

The term “cycloalkyl” refers to a fully saturated cyclic hydrocarbon group containing from 1 to 3 rings and 3 to 8 carbons per ring. Exemplary groups include cyclopropyl, cyclobutyl, cyclopentyl, and cyclohexyl. The term “cycloalkyl” also includes groups having a carbon-carbon bridge of one to two bridgehead carbon atoms, and bicyclic and tricyclic groups in which at least one of the rings is a saturated, carbon-containing ring, in which case the second or third ring may be carbocyclic or heterocyclic, provided that the point of attachment is to the cycloalkyl group. The further rings may be attached to the saturated, carbon-containing ring in a spiro or fused fashion. “Substituted cycloalkyl” refers to a cycloalkyl group substituted with one or more substituents, preferably 1 to 4 substituents, at any available point of attachment. Exemplary substituents include, but are not limited to, alkyl, substituted alkyl, oxo(=O), and those groups recited above as exemplary alkyl substituents.

The term “cycloalkylene” refers to a bivalent cycloalkyl group as defined above. Exemplary groups include cyclopropylene, cyclobutylene, cyclopentylene and cyclohexylene. “Substituted cycloalkylene” refers to a cycloalkylene group substituted with one or more substituents, preferably 1 to 4 substituents, at any available point of attachment selected from those recited for substituted cycloalkyl.

The term “cycloalkenyl” refers to a partially unsaturated cyclic hydrocarbon group containing 1 to 3 rings and 4 to 8 carbons per ring. Exemplary groups include cyclobutenyl, cyclopentenyl, and cyclohexenyl. The term “cycloalkenyl” also includes bicyclic and tricyclic groups in which at least one of the rings is a partially unsaturated, carbon-containing ring and the second or third ring may be carbocyclic or heterocyclic, provided that the point of attachment is to the cycloalkenyl group. “Substituted cycloalkenyl” refers to a cycloalkenyl group substituted with one or more substituents, preferably 1 to 4 substituents, at any available point of attachment selected from those recited above for cycloalkyl groups.

The term “cycloalkenylene” refers to a bivalent cycloalkenyl group, as defined above. Exemplary groups include cyclobutenylene, cyclopentenylene, and cyclohexenylene. “Substituted cycloalkenylene” refers to a cycloalkenylene group substituted with one or more substituents, preferably 1 to 4 substituents, at any available point of attachment, selected from those recited for substituted cycloalkyl.

The terms “alkoxy” or “alkylthio” refer to an alkyl group as described above bonded through an oxygen linkage (—O—) or a sulfur linkage (—S—), respectively. The terms “substituted alkoxy” or “substituted alkylthio” refer to a substituted alkyl group as described above bonded through an oxygen or sulfur linkage, respectively. “Thiol” refers to —SH.

The term “alkoxycarbonyl” refers to an alkoxy group bonded through a carbonyl group (i.e., —C(═O)—O-alkyl).

The term “alkylcarbonyl” refers to an alkyl group bonded through a carbonyl group (i.e., —C(═O)alkyl).

The term “alkylcarbonyloxy” refers to an alkylcarbonyl group bonded through an oxygen linkage (i.e., —O—C(═O)-alkyl).

The term “amido” refers to the group —NHC(═O)H, and amidinyl refers to the group —C(═NH)(NH₂). A “substituted amido” refers to the group —NR^{p}C(═O)R^{q}, and a “substituted amidinyl” refers to the group —C(═NR^{p})(NR^{q}R^{r}), wherein R^{p}, R^{q}, and R^{r} are selected from hydrogen, alkyl, substituted alkyl, cycloalkyl, substituted cycloalkyl, aryl, substituted aryl, heterocyclo, and substituted heterocyclo, provided that at least one of R^{p}, R^{q}, and R^{r} is other than hydrogen.

The term “aryl” encompasses monocyclic and polycyclic aryl groups. The term “monocyclic aryl” refers to phenyl, and the term “polycyclic aryl” refers to napthyl and anthracenyl, to phenyl rings having at least a second ring fused thereto, and to napthyl rings having a third ring fused thereto. In the case of a polycyclic aryl consisting of a phenyl ring having a second or third ring fused thereto, or a napthyl ring having a third ring fused thereto, the additional rings may be aromatic or non-aromatic carbocyclic or heterocyclic rings, provided that in such cases the point of attachment will be to the carbocyclic aromatic ring. Additionally, a ring carbon atom of the second and third further rings may be replaced with a carbonyl [—C(═O)group] (e.g., when such rings are non-aromatic). “Substituted aryl” refers to an aryl group substituted by one or more substituents, preferably 1 to 4 substituents (more preferably 1 or 2), at any point of attachment of any ring, selected from alkyl, substituted alkyl, and the substituents recited above for substituted alkyl groups.

Accordingly, examples of aryl groups include: 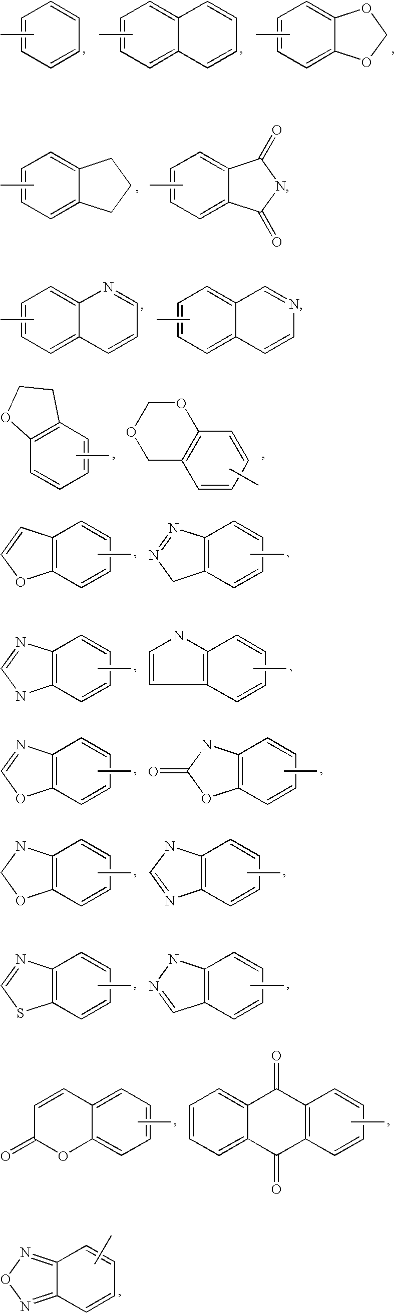
and the like.

The term “arylene” refers to bivalent aryl groups as defined above.

“Carbamoyl” refers to the group —C(═O)—NR^{h}Rⁱ, wherein R^{h} and Rⁱ are selected from hydrogen, alkyl, cycloalkyl, aryl, and heterocyclo.

“Carbamate” refers to the group —O—C(═O)—NR^{h}Rⁱ, and urea refers to the groups NH—C(═O)—NR^{h}Rⁱ and N(alkyl)-C(═O)—NR^{h}Rⁱ′ wherein R^{h} and Rⁱ are selected from the same groups recited for carbamoyl.

“Substituted carbamoyl,” “substituted carbamate,” and “substituted urea” refer to the groups —C(═O)—NR^{h}Rⁱ, —O—C(═O)—NR^{h}Rⁱ, and —N(R^{j})—C(═O)—NR^{h}Rⁱ, respectively, wherein R^{h}, Rⁱ, and R^{j} are selected from hydrogen, alkyl, substituted alkyl, cycloalkyl, substituted cycloalkyl, aryl, substituted aryl, heterocyclo, and substituted heterocyclo, provided that at least one of R^{h}, Rⁱ, and R^{j} is substituted alkyl, substituted cycloalkyl, substituted aryl, or substituted heterocyclo.

The terms “heterocycle”, “heterocyclic” and “heterocyclo” refer to fully saturated, partially unsaturated, or fully unsaturated, including aromatic (i.e., “heteroaryl”) cyclic groups (for example, 3 to 7 membered monocyclic, 7 to 11 membered bicyclic, or 10 to 16 membered tricyclic ring systems) which have at least one heteroatom in at least one carbon atom-containing ring. Thus, the term “heteroaryl” is a subset of heterocyclo groups. Each ring of the heterocyclic group containing a heteroatom may have 1, 2, 3, or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. (The term “heteroarylium” refers to a heteroaryl group bearing a quaternary nitrogen atom and thus a positive charge.) Additionally, one or more (preferably one) carbon rings atoms of the heterocyclo ring may as valence allows be replaced with carbonyl group, i.e., —C(═O)—. The heterocyclic group may be attached to the remainder of the molecule at any heteroatom or carbon atom of the ring or ring system.

Exemplary monocyclic heterocyclic groups include ethylene oxide, azetidinyl, pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, hexahydrodiazepinyl, 4-piperidonyl, pyridyl, pyrazinyl, pyrimidinyl, pyridazinyl, triazinyl, triazolyl, tetrazolyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, and the like.

Exemplary bicyclic heterocyclic groups include indolyl, isoindolyl, benzothiazolyl, benzodioxolyl, benzoxazolyl, benzoxadiazolyl, benzothienyl, quinuclidinyl, quinolinyl, tetrahydroisoquinolinyl, isoquinolinyl, benzimidazolyl, benzopyranyl, indolizinyl, benzofuryl, benzofurazanyl, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]pyridinyl] or furo[2,3-b]pyridinyl), dihydrobenzodioxinyl, dihydrodioxidobenzothiophenyl, dihydroisoindolyl, dihydroindolyl, dihydroquinolinyl, dihydroquinazolinyl (such as 3,4-dihydro-4-oxo-quinazolinyl), triazinylazepinyl, tetrahydroquinolinyl and the like. Exemplary tricyclic heterocyclic groups include carbazolyl, benzidolyl, phenanthrolinyl, dibenzofuranyl, acridinyl, phenanthridinyl, xanthenyl and the like.

The term “heterocyclene” refers to bivalent heterocycle groups as defined above.

“Substituted heterocycle,” “substituted heterocyclic,” and “substituted heterocyclo” (such as “substituted heteroaryl”) refer to heterocycle, heterocyclic or heterocyclo groups substituted with one or more substituents, preferably 1 to 4 substituents, at any available point of attachment, wherein the substituents are selected from those recited above for substituted cycloalkyl groups.

The term “quaternary nitrogen” refers to a tetravalent positively charged nitrogen atom including, for example, the positively charged nitrogen in a tetraalkylammonium group (e.g., tetramethylammonium, N-methylpyridinium), the positively charged nitrogen in protonated ammonium species (e.g., trimethyl-hydroammonium, N-hydropyridinium), the positively charged nitrogen in amine N-oxides (e.g., N-methyl-morpholine-N-oxide, pyridine-N-oxide), and the positively charged nitrogen in an N-amino-ammonium group (e.g., N-aminopyridinium).

The term “heteroaryl” refers to five and six membered monocyclic aromatic heterocyclo groups, as well as bicyclic and tricyclic heterocyclic ring systems in which the point of attachment of the ring system to another group is via a five or six membered aromatic ring of the ring system. Thus, for example, the term heteroaryl includes groups such as five or six membered heteroaryl groups, such as thienyl, pyrrolyl, oxazolyl, pyridyl, pyrazinyl, and the like, wherein fused rings completing bicyclic and tricyclic groups may contain only carbon atoms and may be saturated, partially saturated, or unsaturated. Heteroaryl groups which are bicyclic or tricyclic must include at least one fully aromatic ring but the other fused ring or rings may be aromatic or non-aromatic.

Exemplary monocyclic heteroaryl groups include pyrrolyl, pyrazolyl, imidazolyl, oxazolyl, isoxazolyl, thiazolyl (i.e., 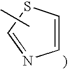
thiadiazolyl, isothiazolyl, furanyl, thienyl, oxadiazolyl, pyridyl, pyrazinyl, pyrimidinyl, and the like.

Exemplary bicyclic heteroaryl groups include indolyl, benzothiazolyl, benzodioxolyl, benzoxaxolyl, benzothienyl, quinolinyl, isoquinolinyl, benzimidazolyl, benzopyranyl, indolizinyl, benzofuranyl, chromonyl, coumarinyl, benzopyranyl, cinnolinyl, quinoxalinyl, indazolyl, pyrrolopyridyl, furopyridinyl, and the like.

Exemplary tricyclic heteroaryl groups include carbazolyl, benzidolyl, phenanthrollinyl, acridinyl, phenanthridinyl, xanthenyl and the like.

The terms “halogen” or “halo” refer to chlorine, bromine, fluorine or iodine.

The terms “hydroxylamine” and “hydroxylamide” refer to the groups —NH—OH and —C(═O)—NH—OH, respectively.

The term “heteroatoms” shall include oxygen, sulfur and nitrogen.

The term “haloalkyl” means an alkyl having one or more halo substituents.

The term “haloalkoxy” means an alkoxy group having one or more halo substituents. For example, “haloalkoxy” includes —OCF₃.

The term “carbocyclic” means a saturated or unsaturated monocyclic or bicyclic ring in which all atoms of all rings are carbon. Thus, the term includes cycloalkyl and aryl rings. The carbocyclic ring may be substituted in which case the substituents are selected from those recited above for cycloalkyl and aryl groups.

When the term “unsaturated” is used herein to refer to a ring or group, the ring or group may be fully unsaturated or partially unsaturated.

When it is stated that a group may be optionally-substituted, this is intended to include unsubstituted groups and substituted groups wherein the substituents are selected from those recited above for the particularly named group. Thus, when reference is made to an optionally substituted aryl, this intended to refer to unsubstituted aryl groups, such as phenyl, or napthyl, and such groups having one or more (preferably 1 to 4, and more preferably 1 or 2), substituents selected from alkyl, substituted alkyl, and those substituents recited for substituted alkyl groups. When the term “optionally substituted” precedes a Markush group, the term “optionally-substituted” is intended to modify each one of the species recited in the Markush group. Thus, for example, the phrase “optionally-substituted aryl, cycloalkyl, or heterocycle” includes aryl, substituted aryl, cycloalkyl, substituted cycloalkyl, heterocycle, and substituted heterocycle.

The term “when m=1, R⁴ and R⁵ may together form a five- or six-membered heterocycle which may have other hetero atoms,” means a five- or six-membered heteroaryl group or saturated heterocycle having from 1 to 3 heteroatoms selected from nitrogen atoms, oxygen atoms and/or sulfur atoms, and it may have oxo groups or the like substituent groups. Examples of such heteroaryl groups include pyrrole, imidazole, pyrazole, pyridine, pyrazine, pyrimidine, pyridazine, triazole and the like, and examples of saturated heterocycles include pyrrolidinyl, piperidinyl, piperazinyl, morpholyl, thiomorpholyl, 1,4-diazepan, thiomorpholine-1-oxide, thiomorpholine-1,1-dioxido, 1,4-oxazepan group and the like. Preferred is a five- or six-membered saturated heterocycle having, in addition to the nitrogen atom to which R⁴ is bonded, one further heteroatom selected from nitrogen, oxygen and sulfur atoms, and more preferred is a thiomorpholino group.

Among the compounds of the invention, in the case of a compound which has a sulfide, the sulfur atom may be converted into oxido at an appropriate oxidation state, and all of these oxido derivatives are included herein.

“N-oxide” refers to compounds wherein the basic nitrogen atom of either a heteroaromatic ring or tertiary amine is oxidized to give a quaternary nitrogen bearing a positive formal charge and an attached oxygen atom bearing a negative formal charge.

“Solvate” refers to a molecular or ionic complex of molecules or ions of solvent with molecules or ions of solute.

When a functional group is termed “protected”, this means that the group is in modified form to mitigate, especially preclude, undesired side reactions at the protected site. Suitable protecting groups for the methods and compounds described herein include, without limitation, those described in standard textbooks, such as Greene, T. W. et al., Protective Groups in Organic Synthesis, Wiley, N.Y. (1991).

When a term such as “(CRR)ₙ” is used, it denotes an optionally substituted alkyl chain existing between the two fragments to which it is bonded, the length of which chain is defined by the range described for the term n. An example of this is n=0-3, implying from zero to three (CRR) units existing between the two fragments, which are attached to the primary and terminal (CRR) units. In the situation where the term n is set to zero (n=0) then a bond exists between the two fragments attached to (CRR).

Unless otherwise indicated, any heteroatom with unsatisfied valences is assumed to have hydrogen atoms sufficient to satisfy the valences.

Carboxylate anion refers to a negatively charged group —COO⁻.

It should be understood that one skilled in the field may make appropriate selections for the substituents for the aryl and heteroaryl groups to provide stable compounds and compounds useful as pharmaceutically-acceptable compounds and/or intermediate compounds useful in making pharmaceutically-acceptable compounds. Thus, for example, in compounds of formula (I), when G is a phenyl ring, preferably the ring will not have three or more (NO₂) groups, and so forth.

Compounds of the present invention may form salts which are also within the scope of this invention. Reference to compounds of Formula I herein is understood to include reference to salts thereof, unless otherwise indicated. The term “salt(s)”, as employed herein, denotes acidic and/or basic salts formed with inorganic and/or organic acids and bases. In addition, when a compound of the present invention contains both a basic moiety, such as but not limited to a pyridine or imidazole, and an acidic moiety such as but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful, e.g., in isolation or purification steps which may be employed during preparation. Salts of the compounds of formula I may be formed, for example, by reacting the involved compound with an amount of acid or base, such as an equivalent amount, in a medium such as one in which the salt precipitates or in an aqueous medium followed by lyophilization.

Compounds of formula I which contain a basic moiety, such as but not limited to an amine or a pyridine or imidazole ring, may form salts with a variety of organic and inorganic acids. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, dodecylsulfates, cyclopentanepropionates, digluconates, ethanesulfonates, glucoheptanoates, fumarates, glycerophosphates, hemisulfates, heptanoates, hexanoates, hydrochlorides, hydrobromides, hydroiodides, hydroxyethanesulfonates lactates, maleates, (e.g., 2-hydroxyethanesulfonates), methanesulfonates, naphthalenesulfonates (e.g., 2-naphthalenesulfonates), nicotinates, nitrates, oxalates, pectinates, persulfates, phenylpropionates (e.g., 3-phenylpropionates), phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

Compounds of formula I which contain an acidic moiety, such as but not limited to a carboxylic acid, may form salts with a variety of organic and inorganic bases. Exemplary basic salts include ammonium salts, alkali metal salts such as sodium, lithium and potassium salts, alkaline earth metal salts such as calcium and magnesium salts, salts with organic bases (for example, organic amines) such as benzathines, dicyclohexylamines, hydrabamines (formed with N,N-bis(dehydroabietyl)ethylenediamine), N-methyl-D-glucamines, N-methyl-D-glycamides, t-butyl amines, and salts with amino acids such as arginine, lysine and the like. Basic nitrogen-containing groups may be quaternized with agents such as lower alkyl halides (e.g. methyl, ethyl, propyl, and butyl chlorides, bromides and iodides), dialkyl sulfates (e.g. dimethyl, diethyl, dibutyl, and diamyl sulfates), long chain halides (e.g. decyl, lauryl, myristyl and stearyl chlorides, bromides and iodides), aralkyl halides (e.g. benzyl and phenethyl bromides), and others.

Prodrugs and solvates of the compounds of the invention are also contemplated herein. The term “prodrug” as employed herein denotes a compound which, upon administration to a subject, undergoes chemical conversion by metabolic or chemical processes to yield a compound of Formula I or a salt, solvate and/or N-oxide thereof. Solvates of the compounds of Formula I include, for example, hydrates.

Compounds of the present invention, and salts thereof, may exist in their tautomeric form (for example, as an amide or imino ether). All such tautomeric forms are contemplated herein as part of the present invention.

All stereoisomers of the present compounds (for example, those which may exist due to asymmetric carbons on various substituents), including enantiomeric forms and diastereomeric forms, are contemplated within the scope of this invention. Individual stereoisomers of the compounds of the invention may, for example, be substantially free of other isomers (e.g., as a pure or substantially pure optical isomer having a specified activity), or may be admixed, for example, as racemates or with all other, or other selected, stereoisomers. The chiral centers of the present invention may have the S or R configuration as defined by the IUPAC 1974 Recommendations. The racemic forms can be resolved by physical methods, such as, for example, fractional crystallization, separation or crystallization of diastereomeric derivatives or separation by chiral column chromatography. The individual optical isomers can be obtained from the racemates by any suitable method, including without limitation, conventional methods, such as, for example, salt formation with an optically active acid followed by crystallization.

All configurational isomers of the compounds of the present invention are contemplated, either in admixture or in pure or substantially pure form. The definition of compounds of the present invention embraces both cis (Z) and trans (E) alkene isomers, as well as cis and trans isomers of cyclic hydrocarbon or heterocyclo rings. As can be appreciated, the preferred configuration can be a function of the particular compound and its preferred activity. Separation of configurational isomers can be achieved by any suitable method, such as column chromatography.

Throughout the specification, groups and substituents thereof may be chosen to provide stable moieties and compounds.

When any variable occurs more than one time in any constituent or in any formula, its definition in each occurrence is independent of its definition at every other occurrence. Combinations of substituents and/or variables are permissible only if such combinations result in stable compounds.

As used herein, “effective amount” refers to an amount of a compound as described herein that may be therapeutically effective to inhibit, prevent or treat the symptoms of a particular disease, disorder or condition. Such diseases, disorders and conditions include, but are not limited to, those pathological conditions associated with NHR's, wherein the treatment or prevention comprises, for example, inhibiting the activity thereof by contacting cells, tissues or receptors with compounds of the present invention.

As used herein, “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio.

### Preferred Compounds

Although the full scope of the invention is set forth above in the Summary of Invention and appended claims, considering equivalents thereof as well, certain compounds are preferred. Accordingly, in one embodiment, preferred compounds of the present invention include pharmaceutically active compounds of formula I: 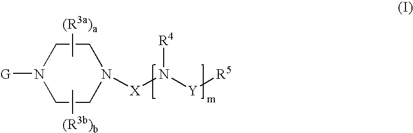
wherein:
G is Ar^{1a}, Ar^{2a}, or Ar^{3a};
Ar^{1a} is a bicyclic or tricyclic aryl or heteroaryl optionally substituted with one to four R⁶
Ar^{2a} is a monocyclic five-membered heteroaryl optionally substituted with one to two R⁶; 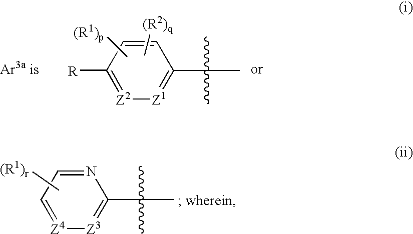
Z¹ and Z² are each independently nitrogen or carbon, the carbon atoms of Z¹ and Z² being bonded to a hydrogen atom or being substituted with a group R¹ or R²;
one of Z³ and Z⁴ is nitrogen and the other of Z³ and Z⁴ is carbon, the carbon atom of Z³ or Z⁴ bonded to a hydrogen atom or being substituted with a group R¹;
R is R², hydrogen, halogen, haloC₁₋₄alkyl, haloC₁₋₄alkoxy, or C₁₋₄alkyl, and additionally, when either (i) p and q taken together are at least two, and/or (ii) q is at least one, then R may also be cyano or nitro, provided that when Z¹ and Z² are carbon and R is cyano, then the carbon atom of Z¹ is substituted with the group R¹ or R²;
R¹ is C₁₋₄alkyl, substituted C₁₋₄alkyl, halo, cyano, nitro, cycloalkyl, substituted cycloalkyl, aryl, substituted aryl, heterocyclo, substituted heterocyclo, —OC(═O)R⁷, —C(═O)—O—R⁷, —C(═O)R⁷, —C(═S)R⁷, —C(═O)NR⁹R¹⁰, —CR¹¹R¹²OR⁷, —OR⁷, —NR⁹R¹⁰, —SR⁷, —S(═O)R⁷, —SO₂R⁷, —SO₂OR⁷, and/or —SO₂NR⁷R⁸;
R² is optionally-substituted aryl, cycloalkyl, or heterocyclo;
R^{3a} and R^{3b} are each independently C₁₋₄alkyl, substituted C₁₋₄alkyl, cycloalkyl, substituted cycloalkyl, heterocyclo, substituted heterocyclo, aryl, substituted aryl, cyano, —OR³, —C(═O)R¹³, —OC(═O)R¹³, —C(═O)OR¹³, —C(═O)NR¹⁴R¹⁵, —SO₂R¹³, —SO₂OR¹³, —SO₂NR¹⁴R¹⁵, and/or a carbamoyl group which may be substituted by 1 or 2 lower alkyl;
X is —C(═O)—;
R⁴ is hydrogen, alkyl, substituted alkyl, cyano, —O-lower alkyl, a carbamoyl group which may be substituted by 1 or 2 lower alkyl; a lower alkylene-C(═O)—, or a lower alkylene-O—C(═O)— group;
Y is a bond, lower alkylene, —C(═O)—, or —SO₂—;
R⁵ is alkyl, substituted alkyl, heterocyclo, substituted heterocyclo, aryl, or substituted aryl;
R⁶ is alkyl, substituted alkyl, alkenyl, substituted alkenyl, alkynyl, substituted alkynyl, halo, cycloalkyl, substituted cycloalkyl, aryl, substituted aryl, heterocyclo, substituted heterocyclo, cyano, nitro, —OC(═O)R⁷, —C(═O)—O—R⁷, —C(═O)R⁷, —C(═S)R⁷, —C(═O)NR⁹R¹⁰, —CR¹¹R¹²OH, —CH₂OR⁷, —OR⁷, —NR⁹R¹⁰, —SR⁷, —S(═O)R⁷, —SO₂R⁷, —SO₂OR⁷, —SO₂NR⁷R⁸, —P(═O)(OR⁷)(OR⁸), —P(═O)(R⁷)(R⁸), and/or —P(═O)(R⁸)(NHR⁹);
R⁷, R⁸ R⁹, R¹⁰, R¹¹, R¹², R¹³, R¹⁴ and R¹⁵ are each independently hydrogen, C₁₋₄alkyl, substituted C₁₋₄alkyl, C₃₋₇cycloalkyl, five or six membered heterocyclo, and/or phenyl;
a and b are both 1;
m is 1;
p and q are 0, 1, and/or 2; and
r is 0, 1, 2, or 3;
or a pharmaceutically acceptable salt, solvate or N-oxide thereof.

According to another aspect of the invention, preferred compounds are those the Formula (I*), 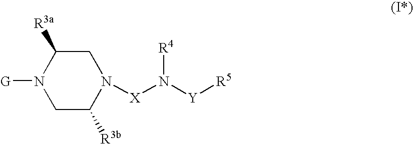
wherein G, X, Y, R^{3a}, R^{3b}, R⁴, R⁵, a and b are as defined above.

According to another aspect of the invention, preferred compounds are those of formula (I), above, wherein selections of G are those optionally-substituted aryl and heteroaryl groups corresponding to the group G in the specific Examples of WO 02/24702, assigned to the present assignee, the entire contents of which is incorporated herein by reference.

According to another aspect of the invention, preferred compounds are those of formula (I), above, wherein R^{3a} and R^{3b} are each independently alkyl or substituted alkyl, more preferably R^{3a} and R^{3b} are both lower alkyl, and most preferred are compounds wherein R^{3a} and R^{3b} are both methyl. More preferred are compounds of Formula (I*), wherein R^{3a} and R^{3b} are both methyl.

In certain embodiments of the compounds of formula I, one of a and b is 1, and the other is 0. In more preferred embodiments, a and b are both 1.

In certain preferred embodiments of the compounds of formula I, R⁴ is H, alkyl or substituted alkyl. More preferred are compounds wherein R⁴ is hydrogen or lower alkyl, and most preferred are compounds wherein R⁴ is H.

In preferred embodiments of the compounds of formula I, R⁵ is an optionally substituted aryl or heteroaryl group.

In certain preferred embodiments of the compounds of formula I, X is —C(═O)—.

In certain embodiments of the compounds of formula I, m is 0 or 1. In more preferred embodiments, m is 1.

In certain embodiments of the compounds of formula I, Y is a bond, lower alkylene, or —C(═O)—. In more preferred embodiments, Y is a bond.

According to another aspect of the invention, preferred compounds are those having the formula, 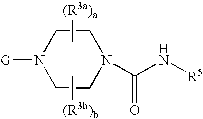
and pharmaceutically-acceptable salts thereof, wherein:
G is Ar^{1a}, Ar^{2a}, or Ar^{3a};
Ar^{1a} is a bicyclic or tricyclic aryl or heteroaryl optionally substituted with one to four R⁶
Ar^{2a} is a monocyclic five-membered heteroaryl optionally substituted with one to two R⁶; 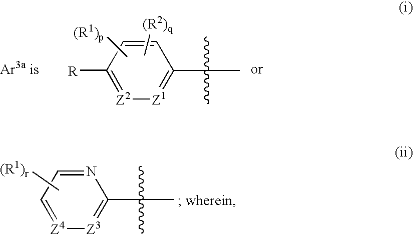
Z¹ and Z² are each independently nitrogen or carbon, the carbon atoms of Z¹ and Z² each being bonded to a hydrogen atom or being substituted with a group R¹ or R²;
one of Z³ and Z⁴ is nitrogen and the other of Z³ and Z⁴ is carbon, the carbon atom of Z³ or Z⁴ being bonded to a hydrogen atom or being substituted with a group R¹;
R is R², hydrogen, halogen, haloC₁₋₄alkyl, haloC₁₋₄alkoxy, C₁₋₄alkyl, cyano, or nitro, provided that if Z¹ and Z² are carbon and R is cyano, then the carbon atom of Z¹ is substituted with the group R¹ or R²;
R¹ is C₁₋₄alkyl, substituted C₁₋₄alkyl, halo, cyano, nitro, cycloalkyl, substituted cycloalkyl, aryl, substituted aryl, heterocyclo, substituted heterocyclo, —OC(═O)R⁷, —C(═O)—O—R⁷, —C(═O)R⁷, —C(═O)NR⁹R¹⁰, —CR¹¹R¹²OR⁷, —OR⁷, —NR⁹R¹⁰, —SR⁷, —S(═O)R⁷, —SO₂R⁷, —SO₂OR⁷, and/or —SO₂NR⁷R⁸;
R² is optionally-substituted aryl, cycloalkyl, and/or heterocyclo;
R⁵ is alkyl, substituted alkyl, phenyl, napthyl, cyclohexyl, O-lower alkylene-phenyl, thienyl, furyl, pyridyl, pyrazinyl, pyrimidinyl, ixosazolyl, thiaziazolyl, benzothiazolyl, benzoimidazolyl, tetrazolyl, morpholinyl, tetrahydrofuryl, thiamorpholinyl, benzofurazanyl, and/or —C(═O)O-(lower alkyl), each of which cyclic groups and alkyl groups of R⁵ may in turn optionally be substituted by one to three groups selected from R¹⁶;
R⁷, R⁸, R⁹, R¹⁰, R¹¹, and R¹² are each independently hydrogen, lower alkyl, and/or substituted lower alkyl;
R¹⁶ is lower alkyl, halo, nitro, trifluoromethyl, trifluoromethoxy, cyano, alkoxy, phenoxy, alkythio, hydroxy, carboxy, alkoxycarbonyl, alkylcarbonyloxy, amino, NR¹⁷R¹⁸, carbamoyl, thiol, phenyl, —C(═O)R¹⁷, —C(═O)NR¹⁷R¹⁸, —NR¹⁷SO₂(alkyl), —SO₂NR¹⁷R¹⁸, —S(═O)alkyl, —SO₂alkyl, —O(lower alkylene)-CF₃, phenyl, and/or morpholinyl;
R¹⁷ and R¹⁸ are independently hydrogen, alkyl, and/or phenyl;
a and b are each 1;
m is 1;
p and q are 0, 1, and/or 2, provided that either p and q taken together are at least two, and/or q is at least one; and
r is 0, 1, 2, or 3.

According to another aspect of the invention, in certain preferred embodiments of the invention, the group G is: 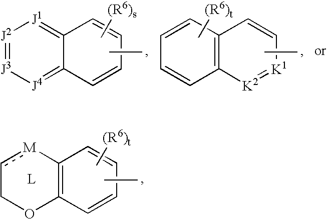
wherein J¹, J², J³ and J⁴ are nitrogen, CH, or CR^{6a}, provided that no more than two of J¹, J², J³, and J⁴ are nitrogen;
K¹ and K² are nitrogen, CH, or CR^{6a};
M is CH, CR^{6a}, nitrogen, NR^{6a}, or oxygen, wherein when M is oxygen or NR⁶, the bond between M and each adjoining carbon atom is a single bond;
R⁶ is attached to any available carbon atom of the bicyclic group G, and R⁶ and R^{6a} are independently alkyl, substituted alkyl, hydroxy, alkoxy, nitro, cyano, halogen, haloalkyl, haloalkoxy, —OC(═O)(alkyl), —C(═O)—O-(alkyl), —C(═O)H, —C(═O)alkyl, —C(═O)NR⁹R¹⁰, —NR⁹R¹⁰, —SH, —S(alkyl), —S(═O)(alkyl), —SO₂(alkyl), and/or —SO₂NR⁹R¹⁰, wherein R⁹ and R¹⁰ are independently hydrogen, alkyl, cycloalkyl, heterocyclo, and/or aryl, and additionally, R⁶ may be oxo when attached to a saturated or partially-saturated ring L;
s is 0, 1, 2, or 3; and
t is 0, 1, 2, 3, or 4.

According to another aspect of the invention, in certain preferred embodiments of the invention, the group G is 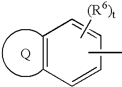
, wherein Q is a fused five membered ring selected from one of: 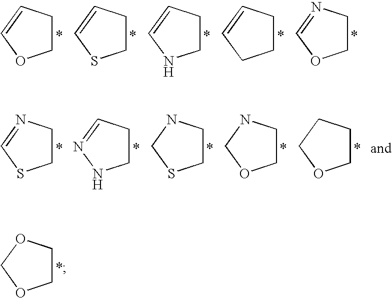
wherein each Q is fused to the phenyl ring along the bond designated with *;
R⁶ is attached to any available carbon atom of the bicyclic group G, including fused ring Q, and is alkyl, substituted alkyl, hydroxy, alkoxy, nitro, cyano, halogen, haloalkyl, haloalkoxy, —OC(═O)(alkyl), —C(═O)—O-(alkyl), —C(═O)H, —C(═O)alkyl, —C(═O)NR⁹R¹⁰, —NR⁹R¹⁰, —SH, —S(alkyl), —S(═O)(alkyl), —SO₂(alkyl), and/or —SO₂NR⁹R¹⁰, wherein R⁹ and R¹⁰ are independently hydrogen, alkyl, cycloalkyl, heterocyclo, and/or aryl, and additionally, where valence allows and R⁶ is attached to ring Q, R⁶ may be oxo; and p1 t is 0, 1, 2, 3, or 4.

According to another aspect of the invention, in certain preferred embodiments of the invention, the group G is: 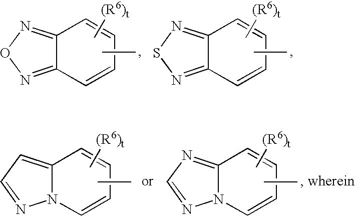
R⁶ is attached to any available carbon atom of the bicyclic group G, and is alkyl, substituted alkyl, hydroxy, alkoxy, nitro, cyano, halogen, haloalkyl, haloalkoxy, —OC(═O)(alkyl), —C(═O)—O-(alkyl), —C(═O)H, —C(═O)alkyl, —C(═O)NR⁹R¹⁰, —NR⁹R¹⁰, —SH, —S(alkyl), —S(═O)(alkyl), —SO₂(alkyl), and/or —SO₂—NR⁹R¹⁰, wherein R⁹ and R¹⁰ are independently hydrogen, alkyl, cycloalkyl, heterocyclo, and/or aryl; and
t is 0, 1, 2, 3, or 4.

According to another aspect of the invention, in certain preferred embodiments of the invention, the group G is: 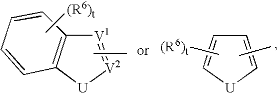
wherein
U is S, O, S(═O), S(O)₂, NH, or NR^{6a};
one of V¹ and V² is nitrogen and the other of V¹ and V² is carbon, the group G being attached to the piperazinyl ring via the atom V¹ or V² that is carbon;
R⁶ is attached to any available carbon atom of the group G, and R⁶ and R^{6a} are independently alkyl, hydroxy, alkoxy, nitro, cyano, halogen, haloalkyl, haloalkoxy, —OC(═O)(alkyl), —C(═O)—O-(alkyl), —C(═O)H, —C(═O)alkyl, —C(═O)NR⁹R¹⁰, —NR⁹R¹⁰, —SH, —S(alkyl), —S(═O)(alkyl), —SO₂(alkyl), and/or —SO₂—NR⁹R¹⁰, wherein R⁹ and R¹⁰ are independently hydrogen, alkyl, cycloalkyl, heterocyclo, and/or aryl; and
t is 0, 1, 2, 3, or 4.

According to another aspect of the invention, also preferred are compounds having the formula, 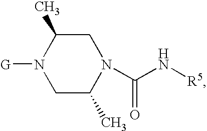
wherein,
R⁵ is an aryl or heteroaryl group optionally substituted with one to three groups selected from R¹⁶;
R¹⁶ is lower alkyl, halo, nitro, trifluoromethyl, trifluoromethoxy, cyano, alkoxy, phenoxy, alkythio, hydroxy, carboxy, alkoxycarbonyl, alkylcarbonyloxy, amino, NR¹⁷R¹⁸, carbamoyl, thiol, phenyl, —C(═O)R¹⁷, —C(═O)NR¹⁷R¹⁸, —NR¹⁷SO₂(alkyl), —SO₂NR¹⁷R¹⁸, —S(═O)alkyl, —SO₂alkyl, —O(lower alkylene)-CF₃, phenyl, and/or morpholinyl;
R¹⁷ and R¹⁸ are independently hydrogen, alkyl, and/or phenyl; and
G is an optionally substituted bicyclic aryl or heteroaryl.

Further preferred are compounds as immediately described above, wherein G is a more particularly preferred bicyclic aryl or heteroaryl as identified herein, e.g., as recited in certain dependent claims herein or as set forth in the specific examples.

Embodiments indicated herein as exemplary or preferred are intended to be illustrative and not limiting.

### Methods of Preparation

The compounds of the present invention may be prepared by methods such as those illustrated in the following Schemes. Solvents, temperatures, pressures, and other reaction conditions may readily be selected by one of ordinary skill in the art. Starting materials are commercially available or readily prepared by one of ordinary skill in the art. Combinatorial techniques may be employed in the preparation of compounds, for example, where the intermediates possess groups suitable for these techniques. See the following which describe other methods which may be employed in the preparation of compounds of the present invention: Williams, V. E.; Lemieux, R. P.; J. Chem. Soc., Chem. Commun. 1996, 19, 2259; Marcoux, J.-F; Wagaw S.; Buchwald, S. L.; J. Org. Chem. 1997, 62, 1568; Lovell, J. M.; Joule, J. A.; J. Chem. Soc., Perkin Trans. 1 1996, 19, 2391; Ishiyama, T.; Murata, M.; Miyaura, N.; J. Org. Chem. 1995, 60, 7508; Nakamura, H.; Fujiwara, M.; Yamamoto, Y.; Bull. Chem. Soc. Jpn. 2000, 73, 231; Lam, P. Y. S.; Clark, C. G.; Saubern, S.; Adams, J.; Averill, K. M.; Chan, D. M. T.; Combs, A.; Synlett 2000, 5, 674.

All documents cited in the present specification, such as those cited in this “Methods of Preparation” as well as other sections herein, are incorporated herein by reference in their entirety. Reference to any document herein is not to be construed as an admission that such document is prior art.

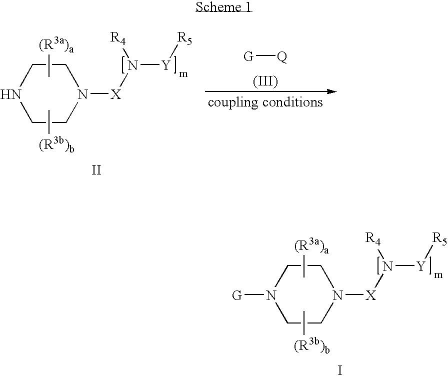

Generally, compounds of formula I may be made as illustrated in Scheme 1. Reaction of a compound of formula II, wherein X, Y, a, b, m, R^{3a}, R^{3b}, R⁴ and R⁵ are as previously defined, with an appropriately substituted G-Q compound III, wherein G is as previously defined and Q is a halogen, preferably Br, or B(OH)₂, under suitable amine coupling conditions provides compounds of formula I wherein X, Y, a, b, m, G, R^{3a}, R^{3b}, R⁴ and R⁵ are as previously defined.

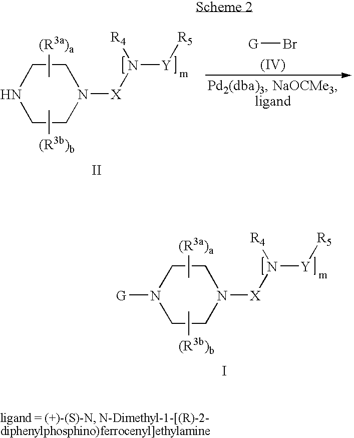

As illustrated in Scheme 2, compounds of formula I can be prepared by reaction of a compound of formula II, wherein X, Y, a, b, m, R^{3a}, R^{3b}, R⁴ and R⁵ are as previously described, with a compound of formula IV, wherein G is as previously described. Reaction of II and IV in a solvent such as toluene with a palladium catalyst such as Pd₂(dba)₃ and a base such as sodium tert-butoxide in the presence of a ligand such as (+)-(S)—N,N-dimethyl-1-[(R)-2-(diphenylphosphino)-ferrocenyl]-ethylamine provides a compound of formula I wherein X, Y, a, b, m, G, R^{3a}, R^{3b}, R⁴ and R⁵ are as previously described.

A compound of formula IV can be obtained from commercial sources or readily made by one skilled in the art, for example, in accordance with Williams, V. E.; Lemieux, R. P.; J. Chem. Soc., Chem. Commun. 1996, 19, 2259, and the references found therein.

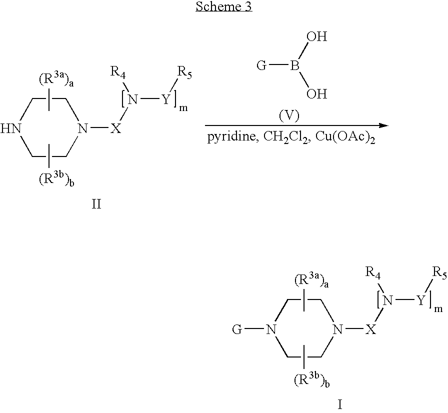

Compounds of formula I may also be prepared as illustrated in Scheme 3. A compound of formula II, wherein X, Y, a, b, m, R^{3a}, R^{3b}, R⁴, and R⁵ are as previously described, may be reacted with a compound of formula V, wherein G is as previously described, using a copper (II) reagent such as Cu(OAc)₂, in a solvent such as DCM using a base such a pyridine as described in Lam et al., Synlett 2000, 5, 674, and Marcoux, et al., J. Org. Chem. 1997, 62, 1568 (and references contained therein).

Compounds of formula VI can be obtained from commercial sources or may be readily prepared by one skilled in the art, for example, in accordance with Lovell, J. M.; Joule, J. A.; J. Chem. Soc., Perkin Trans. 1 1996, 19, 2391; Ishiyama, T.; Murata, M.; Miyaura, N.; J. Org. Chem. 1995, 60, 7508-7510; and Nakamura, H.; Fujiwara, M.; Yamamoto, Y.; Bull. Chem. Soc. Jpn. 2000, 73, 231, and the references cited therein.

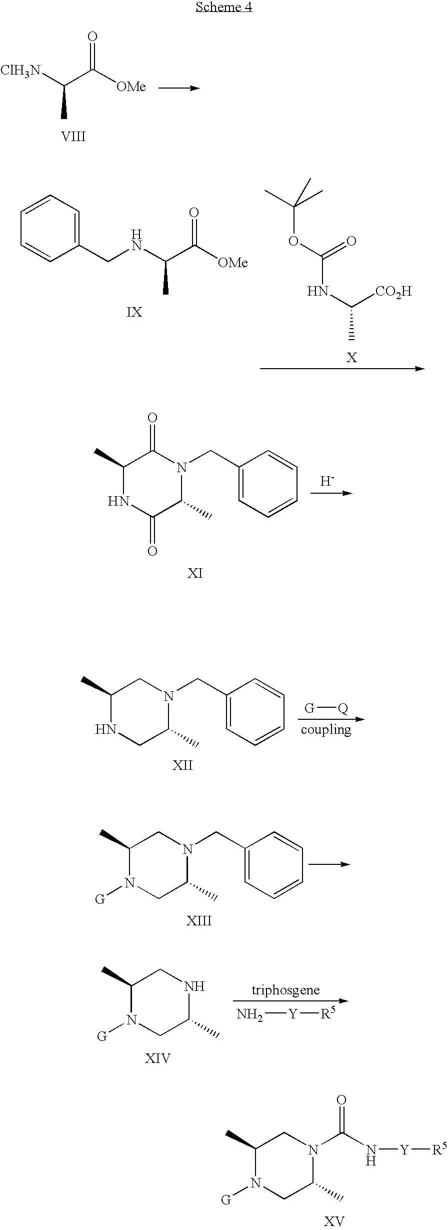

Compounds of formula XV, which are compounds of formula I wherein X is —C(═O)—, a and b are 1, R^{3a} is methyl, R^{3b} is methyl, R⁴ is H, m is 1, and G, Y, and R⁵ are as previously defined may be prepared according to Scheme 4.

Protection of D-alanine methyl ester hydrochloride with benzaldehyde and triethyl amine in a solvent such as THF, followed by reduction with NaBH₄ in NaOH, yields a compound of formula IX. Subsequent reaction with (2R)-2-benzylamino-propionic acid methyl ester and DCC in a solvent such as DCM at reduced temperature, followed by deprotection of the Boc group with TFA in DCM, provides a compound of formula XI. Reaction of a compound of formula XI with a reducing agent such as LiAlH₄ in a solvent such as THF then provides compound XII.

Coupling of compound XII with a compound of formula G-Q, wherein G is as previously described and Q is a halogen, preferably Br, or B(OH)₂, can be accomplished using conditions as described in Schemes 2 or 3 to provide a compound of formula XIII in accordance with Marcoux et al., J. Org. Chem. 1997, 62, 1568; Lam et al., Synlett 2000, 5, 674, and Lemieux, R. P.; J. Chem. Soc., Chem. Commun. 1996, 19, 2259; and the references described therein.

Deprotection of the compound XIII to afford compound XIV can be accomplished by treatment of compound XIII with 1-chloroethyl chloroformate in a solvent such as dichloroethane or MeOH, with heating. Subsequent reaction of compound XIV with triphosgene and NH₂—Y—R⁵, wherein Y and R⁵ are as previously described, affords compound XV which is a compound of formula I wherein X is —C(═O)—, a and b are 1, R^{3a} is methyl, R^{3b} is methyl, R⁴ is H, m is 1, and G, Y, and R⁵ are as previously defined.

The aforementioned approach(es) can be applied in a combinatorial fashion, for example, by utilizing a multi-well reaction block such as is described in Waldemar Ruediger, Wen-Jeng Li, John W., Allen Jr., and Harold N. Weller III, U.S. Pat. No. 5,961,925, Apparatus for Synthesis of Multiple Organic Compounds With Pinch Valve Block (incorporated herein by reference in its entirety). By utilizing the above-mentioned multi-well reaction block, one can, for example, perform multiples of 96 reactions at a time. Solvent can then be removed from the reaction tubes without removal from the reaction block and the crude products can be precipitated using a base such as sodium bicarbonate. The precipitates can be collected by filtration of the reaction block and then the desired products can be transferred directly to 96 well plates for screening. In this fashion, a large array of compounds of formula I can be synthesized, and tests conducted as desired by an automated approach.

### Use and Utility

Compounds of the present invention modulate the function of nuclear hormone receptors (NHR), and include compounds which are, for example, agonists, partial agonists, antagonists, or partial antagonists of the androgen receptor (AR), the estrogen receptor (ER), the progesterone receptor (PR), the glucocorticoid receptor (GR), the mineralocorticoid receptor (MR), the steroid and xenobiotic receptor (SXR), other steroid-binding NHR's, the Orphan receptors, or other NHR's. Selective modulation of one such NHR relative to others within the NHR family is preferred. “Modulation” includes, for example, activation (e.g., agonist activity such as selective androgen receptor agonist activity) or inhibition (e.g., antagonist activity).

The present compounds may thus be useful in the treatment of NHR-associated conditions. A “NHR-associated condition”, as used herein, denotes a condition or disorder which can be treated by modulating the function of a NHR in a subject, wherein treatment comprises prevention (e.g., prophylactic treatment), partial alleviation, or cure of the condition or disorder. Modulation may occur locally, for example, within certain tissues of the subject, or more extensively throughout a subject being treated for such a condition disorder.

The compounds of the present invention may be useful for the treatment of a variety of conditions and disorders including, but not limited to, those described following:

Compounds of Formula I may be applied as agonists, partial agonists, antagonists, or partial antagonists of the estrogen receptor, preferably selectively to that receptor, in an array of medical conditions which involve modulation of the estrogen receptor pathway. Applications of said compounds include but are not limited to: osteoporosis, hot flushes, vaginal dryness, prostate cancer, breast cancer, endometrial cancer, cancers expressing the estrogen receptor such as the aforementioned cancers and others, contraception, pregnancy termination, menopause, amennoreahea, and dysmennoreahea.

Compounds of Formula I may be applied as agonists, partial agonists, antagonists, or partial antagonists of the progesterone receptor, preferably selectively to that receptor, in an array of medical conditions which involve modulation of the progesterone receptor pathway. Applications of said compounds include but are not limited to: breast cancer, other cancers containing the progesterone receptor, endometriosis, cachexia, contraception, menopause, cyclesynchrony, meniginoma, dysmennoreahea, fibroids, pregnancy termination, labor induction, and osteoporosis.

Compounds of Formula I may be applied as agonists, partial agonists, antagonists, or partial antagonists of the glucocorticoid receptor, preferably selectively to that receptor, in an array of medical conditions which involve modulation of the glucocorticoid receptor pathway. Applications of said compounds include but are not limited to: inflammatory diseases, autoimmune diseases, prostate cancer, breast cancer, Alzheimer's disease, psychotic disorders, drug dependence, non-insulin dependent Diabetes Mellitus, and as dopamine receptor blocking agents or otherwise as agents for the treatment of dopamine receptor mediated disorders.

Compounds of the present invention may be applied as agonists, partial agonists, antagonists, or partial antagonists of the mineralocorticoid receptor, preferably selectively to that receptor, in an array of medical conditions which involve modulation of the mineralocorticoid receptor pathway. Applications of said compounds include but are not limited to: drug withdrawal syndrome and inflammatory diseases.

Compounds of the present invention may be applied as agonists, partial agonists, antagonists, or partial antagonists of the aldosterone receptor, preferably selectively to that receptor, in an array of medical conditions which involve modulation of the aldosterone receptor pathway. One application of said compounds includes but is not limited to: congestive heart failure.

Compounds of Formula I may be applied as agonists, partial agonists, antagonists, or partial antagonists of the androgen receptor, preferably selectively to that receptor, in an array of medical conditions which involve modulation of the androgen receptor pathway. Applications of said compounds include but are not limited to: hirsutism, acne, seborrhea, Alzheimer's disease, androgenic alopecia, hypogonadism, hyperpilosity, benign prostate hypertrophia, adenomas and neoplasies of the prostate (such as advanced metastatic prostate cancer), treatment of benign or malignant tumor cells containing the androgen receptor such as is the case for breast, brain, skin, ovarian, bladder, lymphatic, liver and kidney cancers, pancreatic cancers, modulation of VCAM expression and applications therein for the treatment of heart disease, inflammation and immune modulations, modulation of VEGF expression and the applications therein for use as antiangiogenic agents, osteoporosis, suppressing spermatogenesis, libido, cachexia, endometriosis, polycystic ovary syndrome, anorexia, androgen supplement for age related decreased testosterone levels in men, male menopause, male hormone replacement, male and female sexual dysfunction, and inhibition of muscular atrophy in ambulatory patients. For example, pan AR modulation is contemplated, with prostate selective AR modulation (“SARM”) being particularly preferred, such as for the treatment of early stage prostate cancers.

Compounds of Formula I may be applied as (preferably, selective) antagonists of the mutated androgen receptor, for example, found in many tumor lines. Examples of such mutants are those found in representative prostate tumor cell lines such as LNCap, (T877A mutation, Biophys. Acta, 187, 1052 (1990)), PCa2b, (L701H & T877A mutations, J. Urol., 162, 2192 (1999)) and CWR22, (H874Y mutation, Mol. Endo., 11, 450 (1997)). Applications of said compounds include but are not limited to: adenomas and neoplasies of the prostate, breast cancer, and endometrial cancer.

Compounds of Formula I may be applied as agonists, partial agonists, antagonists, or partial antagonists of the steroid and xenobiotic receptor, preferably selectively to that receptor, in an array of medical conditions which involve modulation of the steroid and xenobiotic receptor pathway. Applications of said compounds include but are not limited to: treatment of disregulation of cholesterol homeostasis and attenuation of metabolism of pharmaceutical agents by co-administration of an agent (compound of the present invention) which modulates the P450 regulator effects of SXR.

Along with the aforementioned NHR, there also exist a number of NHR for which the activating or deactivating ligands may not be characterized. These proteins are classified as NHR due to strong sequence homology to other NHR, and are known as the Orphan receptors. Because the Orphan receptors demonstrate strong sequence homology to other NHR, compounds of the present invention include those which serve as modulators of the function of the Orphan NHR. Orphan receptors which are modulated by NHR modulators such as compounds within the scope of the present invention are exemplified, but not limited to, those listed in Table A. Exemplary therapeutic applications of modulators of said Orphan receptors are also listed in Table A, but are not limited to the examples therein.

TABLE A
Exemplary Orphan nuclear hormone receptors, form (M =
monomeric, D = heterodimeric, H = homodimeric), tissue
expression and target therapeutic applications. (CNS =
central nervous system)

| | | | Target Therapeutic |
|---|---|---|---|
| Receptor | Form | Tissue Expression | Application |
| NURR1 | M/D | Dopaminergic Neurons | Parkinson's Disease |
| RZRβ | M | Brain (Pituitary), Muscle | Sleep Disorders |
| RORα | M | Cerebellum, Purkinje | Arthritis, Cerebellar |
| | | Cells | Ataxia |
| NOR-1 | M | Brain, Muscle, Heart, | CNS Disorders, Cancer |
| | | Adrenal, Thymus | |
| NGFI-Bβ | M/D | Brain | CNS Disorders |
| COUP-Tfα | H | Brain | CNS Disorders |
| COUP-TFβ | H | Brain | CNS Disorders |
| COUP-TFγχ | H | Brain | CNS Disorders |
| Nur77 | H | Brain, Thymus, Adrenals | CNS Disorders |
| Rev-ErbAα | H | Muscle, Brain | Obesity |
| | | (Ubiquitous) | |
| HNF4α | H | Liver, Kidney, Intestine | Diabetes |
| SF-1 | M | Gonads, Pituitary | Metabolic Disorders |
| LXRα, β | D | Kidney (Ubiquitous) | Metabolic Disorders |
| GCNF | M/H | Testes, Ovary | Infertility |
| ERRα, β | M | Placenta, Bone | Infertility, Osteoporosis |
| FXR | D | Liver, Kidney | Metabolic Disorders |
| CARα | H | Liver, Kidney | Metabolic Disorders |
| PXR | H | Liver, Intestine | Metabolic Disorders |
| COUP-TF2 | D | Testis | Oncology/angiogenesis |
| (ARP1) | | | |
| RORbeta | M | CNS, retina, pineal gland | Metabolic Disorders |

The present invention thus provides methods for the treatment of NHR-associated conditions, comprising the step of administering to a subject in need thereof at least one compound of formula I in an amount effective therefor. Other therapeutic agents such as those described below may be employed with the inventive compounds in the present methods (for example, separately, or formulated together as a fixed dose). In the methods of the present invention, such other therapeutic agent(s) may be administered prior to, simultaneously with, or following the administration of the compound(s) of the present invention.

The present invention also provides pharmaceutical compositions comprising at least one of the compounds of formula I capable of treating a NHR-associated condition in an amount effective therefor, and a pharmaceutically acceptable carrier (vehicle or diluent). The compositions of the present invention can contain other therapeutic agents as described below, and can be formulated, for example, by employing conventional solid or liquid vehicles or diluents, as well as pharmaceutical additives of a type appropriate to the mode of desired administration (for example, excipients, binders, preservatives, stabilizers, flavors, etc.) according to techniques such as those well known in the art of pharmaceutical formulation.

It should be noted that the compounds of the present invention, without limitation as to their mechanism of action, may be useful in treating any of the conditions or disorders listed or described herein such as inflammatory diseases or cancers, or other proliferate diseases, and in compositions for treating such conditions or disorders. Such conditions and disorders include, without limitation, any of those described previously, as well as those described following such as: maintenance of muscle strength and function (e.g., in the elderly); reversal or prevention of frailty or age-related functional decline (“ARFD”) in the elderly (e.g., sarcopenia); treatment of catabolic side effects of glucocorticoids; prevention and/or treatment of reduced bone mass, density or growth (e.g., osteoporosis and osteopenia); treatment of chronic fatigue syndrome (CFS); chronic malagia; treatment of acute fatigue syndrome and muscle loss following elective surgery (e.g., post-surgical rehabilitation); acceleration of wound healing; accelerating bone fracture repair (such as accelerating the recovery of hip fracture patients); accelerating healing of complicated fractures, e.g. distraction osteogenesis; in joint replacement; prevention of post-surgical adhesion formation; acceleration of tooth repair or growth; maintenance of sensory function (e.g., hearing, sight, olefaction and taste); treatment of periodontal disease; treatment of wasting secondary to fractures and wasting in connection with chronic obstructive pulmonary disease (COPD), chronic liver disease, AIDS, weightlessness, cancer cachexia, burn and trauma recovery, chronic catabolic state (e.g., coma), eating disorders (e.g., anorexia) and chemotherapy; treatment of cardiomyopathy; treatment of thrombocytopenia; treatment of growth retardation in connection with Crohn's disease; treatment of short bowel syndrome; treatment of irritable bowel syndrome; treatment of inflammatory bowel disease; treatment of Crohn's disease and ulcerative colitis; treatment of complications associated with transplantation; treatment of physiological short stature including growth hormone deficient children and short stature associated with chronic illness; treatment of obesity and growth retardation associated with obesity; treatment of anorexia (e.g., associated with cachexia or aging); treatment of hypercortisolism and Cushing's syndrome; Paget's disease; treatment of osteoarthritis; induction of pulsatile growth hormone release; treatment of osteochondrodysplasias; treatment of depression, nervousness, irritability and stress; treatment of reduced mental energy and low self-esteem (e.g., motivation/assertiveness); improvement of cognitive function (e.g., the treatment of dementia, including Alzheimer's disease and short term memory loss); treatment of catabolism in connection with pulmonary dysfunction and ventilator dependency; treatment of cardiac dysfunction (e.g., associated with valvular disease, myocardial infarction, cardiac hypertrophy or congestive heart failure); lowering blood pressure; protection against ventricular dysfunction or prevention of reperfusion events; treatment of adults in chronic dialysis; reversal or slowing of the catabolic state of aging; attenuation or reversal of protein catabolic responses following trauma (e.g., reversal of the catabolic state associated with surgery, congestive heart failure, cardiac myopathy, burns, cancer, COPD etc.); reducing cachexia and protein loss due to chronic illness such as cancer or AIDS; treatment of hyperinsulinemia including nesidioblastosis; treatment of immunosuppressed patients; treatment of wasting in connection with multiple sclerosis or other neurodegenerative disorders; promotion of myelin repair; maintenance of skin thickness; treatment of metabolic homeostasis and renal homeostasis (e.g., in the frail elderly); stimulation of osteoblasts, bone remodeling and cartilage growth; regulation of food intake; treatment of insulin resistance, including NIDDM, in mammals (e.g., humans); treatment of insulin resistance in the heart; improvement of sleep quality and correction of the relative hyposomatotropism of senescence due to high increase in REM sleep and a decrease in REM latency; treatment of hypothermia; treatment of congestive heart failure; treatment of lipodystrophy (e.g., in patients taking HIV or AIDS therapies such as protease inhibitors); treatment of muscular atrophy (e.g., due to physical inactivity, bed rest or reduced weight-bearing conditions); treatment of musculoskeletal impairment (e.g., in the elderly); improvement of the overall pulmonary function; treatment of sleep disorders; and the treatment of the catabolic state of prolonged critical illness; treatment of hirsutism, acne, seborrhea, androgenic alopecia, anemia, hyperpilosity, benign prostate hypertrophy, adenomas and neoplasies of the prostate (e.g., advanced metastatic prostate cancer) and malignant tumor cells containing the androgen receptor, such as is the case for breast, brain, skin, ovarian, bladder, lymphatic, liver and kidney cancers; cancers of the skin, pancreas, endometrium, lung and colon; osteosarcoma; hypercalcemia of malignancy; metastatic bone disease; treatment of spermatogenesis, endometriosis and polycystic ovary syndrome; counteracting preeclampsia, eclampsia of pregnancy and preterm labor; treatment of premenstrual syndrome; treatment of vaginal dryness; age related decreased testosterone levels in men, male menopause, hypogonadism, male hormone replacement, male and female sexual dysfunction (e.g., erectile dysfunction, decreased sex drive, sexual well-being, decreased libido), male and female contraception, hair loss, Reaven's Syndrome and the enhancement of bone and muscle performance/strength; and the conditions, diseases, and maladies collectively referenced to as “Syndrome X” or Metabolic Syndrome as detailed in Johannsson J. Clin. Endocrinol. Metab., 82, 727-34 (1997).

The present compounds may have therapeutic utility in the modulation of immune cell activation/proliferation, e.g., as competitive inhibitors of intercellular ligand/receptor binding reactions involving CAMs (Cellular Adhesion Molecules) and Leukointegrins. For example, the present compounds may modulate LFA-ICAM 1, and may be particularly useful as LFA-ICAM 1 antagonists, and in the treatment of all conditions associated with LFA-ICAM 1 such as immunological disorders. Preferred utilities for the present compounds include, but are not limited to: inflammatory conditions such as those resulting from a response of the non-specific immune system in a mammal (e.g., adult respiratory distress syndrome, shock, oxygen toxicity, multiple organ injury syndrome secondary to septicemia, multiple organ injury syndrome secondary to trauma, reperfusion injury of tissue due to cardiopulmonary bypass, myocardial infarction or use with thrombolysis agents, acute glomerulonephritis, vasculitis, reactive arthritis, dermatosis with acute inflammatory components, stroke, thermal injury, hemodialysis, leukapheresis, ulcerative colitis, necrotizing enterocolitis and granulocyte transfusion associated syndrome) and conditions resulting from a response of the specific immune system in a mammal (e.g., psoriasis, organ/tissue transplant rejection, graft vs. host reactions and autoimmune diseases including Raynaud's syndrome, autoimmune thyroiditis, dermatitis, multiple sclerosis, rheumatoid arthritis, insulin-dependent diabetes mellitus, uveitis, inflammatory bowel disease including Crohn's disease and ulcerative colitis, and systemic lupus erythematosus). The present compounds can be used in treating asthma or as an adjunct to minimize toxicity with cytokine therapy in the treatment of cancers. The present compounds can be employed in the treatment of all diseases currently treatable through steroid therapy. The present compounds may be employed for the treatment of these and other disorders alone or with other immunosuppressive or antiinflammatory agents. In accordance with the invention, a compound of formula I can be administered prior to the onset of inflammation (so as to suppress an anticipated inflammation) or after the initiation of inflammation. When provided prophylactically, the immunosupressive compound(s) are preferably provided in advance of any inflammatory response or symptom (for example, prior to, at, or shortly after the time of an organ or tissue transplant but in advance of any symptoms or organ rejection). The prophylactic administration of a compound of the formula I may prevent or attenuate any subsequent inflammatory response (such as, for example, rejection of a transplanted organ or tissue, etc.) Administration of a compound of formula I may attenuate any actual inflammation (such as, for example, the rejection of a transplanted organ or tissue).

The compounds of the present invention may be administered for any of the uses described herein by any suitable means, for example, orally, such as in the form of tablets, capsules, granules or powders; sublingually; bucally; parenterally, such as by subcutaneous, intravenous, intramuscular, or intrasternal injection or infusion techniques (e.g., as sterile injectable aqueous or non-aqueous solutions or suspensions); nasally, including administration to the nasal membranes, such as by inhalation spray; topically, such as in the form of a cream or ointment; or rectally such as in the form of suppositories; in dosage unit formulations containing non-toxic, pharmaceutically acceptable vehicles or diluents. The present compounds may, for example, be administered in a form suitable for immediate release or extended release. Immediate release or extended release may be achieved by the use of suitable pharmaceutical compositions comprising the present compounds, or, particularly in the case of extended release, by the use of devices such as subcutaneous implants or osmotic pumps. The present compounds may also be administered liposomally.

Exemplary compositions for oral administration include suspensions which may contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which may contain, for example, microcrystalline cellulose, dicalcium phosphate, starch, magnesium stearate and/or lactose and/or other excipients, binders, extenders, disintegrants, diluents, and lubricants such as those known in the art. The compounds of Formula I may also be delivered through the oral cavity by sublingual and/or buccal administration. Molded tablets, compressed tablets, or freeze-dried tablets are exemplary forms which may be used. Exemplary compositions include those formulating the present compound(s) with fast dissolving diluents such as mannitol, lactose, sucrose, and/or cyclodextrins. Also included in such formulations may be high molecular weight excipients such as celluloses (avicel) or polyethylene glycols (PEG). Such formulations may also include an excipient to aid mucosal adhesion such as hydroxy propyl cellulose (HPC), hydroxy propyl methyl cellulose (HPMC), sodium carboxy methyl cellulose (SCMC), maleic anhydride copolymer (e.g., Gantrez), and agents to control release such as polyacrylic copolymer (e.g. Carbopol 934). Lubricants, glidants, flavors, coloring agents, and stabilizers may also be added for ease of fabrication and use.

Exemplary compositions for nasal aerosol or inhalation administration include solutions in saline which may contain, for example, benzyl alcohol or other suitable preservatives, absorption promoters to enhance bioavailability, and/or other solubilizing or dispersing agents such as those known in the art.

Exemplary compositions for parenteral administration include injectable solutions or suspensions which may contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid, or Cremaphor.

Exemplary compositions for rectal administration include suppositories which may contain, for example, a suitable non-irritating excipient, such as cocoa butter, synthetic glyceride esters or polyethylene glycols, which are solid at ordinary temperatures, but liquefy and/or dissolve in the rectal cavity to release the drug.

Exemplary compositions for topical administration include a topical carrier such as Plastibase (mineral oil gelled with polyethylene).

The effective amount of a compound of the present invention may be determined by one of ordinary skill in the art, and would be apparent to one of ordinary skill in the art, once armed with the teaching in the present disclosure. Generally, small dosages may be used initially and, if necessary, increased by small increments until the desired effect under the circumstances is reached. Generally speaking, oral administration may require higher dosages.

Effective amounts of the present compounds include exemplary dosage amounts for an adult human of from about 1 to about 100 (for example, about 15 or lower, especially about 1 to about 3 or less) mg/kg of body weight of active compound per day, which may be administered in a single dose or in the form of individual divided doses, such as from 1 to 4 times per day. It will be understood that the specific dose level and frequency of dosage for any particular subject may be varied and will depend upon a variety of factors including the activity of the specific compound employed, the metabolic stability and length of action of that compound, the species, age, body weight, general health, sex and diet of the subject, the mode and time of administration, rate of excretion, drug combination, and severity of the particular condition. Preferred subjects for treatment include animals, most preferably mammalian species such as humans, and domestic animals such as dogs, cats and the like, subject to NHR-associated conditions.

As mentioned above, the compounds of the present invention may be employed alone or in combination with each other and/or other suitable therapeutic agents useful in the treatment of NHR-associated conditions, e.g., an antibiotic or other pharmaceutically active material.

For example, the compounds of the present invention may be combined with growth promoting agents, such as, but not limited to, TRH, diethylstilbesterol, theophylline, enkephalins, E series prostaglandins, compounds disclosed in U.S. Pat. No. 3,239,345, e.g., zeranol, and compounds disclosed in U.S. Pat. No. 4,036,979, e.g., sulbenox or peptides disclosed in U.S. Pat. No. 4,411,890.

The compounds of the invention may also be used in combination with growth hormone secretagogues such as GHRP-6, GHRP-1 (as described in U.S. Pat. No. 4,411,890 and publications WO 89/07110 and WO 89/07111), GHRP-2 (as described in WO 93/04081), NN703 (Novo Nordisk), LY444711 (Lilly), MK-677 (Merck), CP424391 (Pfizer) and B-HT920, or with growth hormone releasing factor and its analogs or growth hormone and its analogs or somatomedins including IGF-1 and IGF-2, or with alpha-adrenergic agonists, such as clonidine or serotinin 5-HT_{D} agonists, such as sumatriptan, or agents which inhibit somatostatin or its release, such as physostigmine and pyridostigmine. A still further use of the disclosed compounds of the invention is in combination with parathyroid hormone, PTH(1-34) or bisphosphonates, such as MK-217 (alendronate).

A still further use of the compounds of the invention is in combination with estrogen, testosterone, a selective estrogen receptor modulator, such as tamoxifen or raloxifene, or other androgen receptor modulators, such as those disclosed in Edwards, J. P. et al., Bio. Med. Chem. Let., 9, 1003-1008 (1999) and Hamann, L. G. et al., J. Med. Chem., 42, 210-212 (1999).

A further use of the compounds of this invention is in combination with progesterone receptor agonists (“PRA”), such as levonorgestrel or medroxyprogesterone acetate (MPA).

The compounds of the present invention may be employed alone or in combination with each other and/or other modulators of nuclear hormone receptors or other suitable therapeutic agents useful in the treatment of the aforementioned disorders including: anti-diabetic agents; anti-osteoporosis agents; anti-obesity agents; anti-inflammatory agents; anti-anxiety agents; anti-depressants; anti-hypertensive agents; anti-platelet agents; anti-thrombotic and thrombolytic agents; cardiac glycosides; cholesterol/lipid lowering agents; mineralocorticoid receptor antagonists; phospodiesterase inhibitors; protein tyrosine kinase inhibitors; thyroid mimetics (including thyroid receptor agonists); anabolic agents; HIV or AIDS therapies; therapies useful in the treatment of Alzheimer's disease and other cognitive disorders; therapies useful in the treatment of sleeping disorders; anti-proliferative agents; and anti-tumor agents.

Examples of suitable anti-diabetic agents for use in combination with the compounds of the present invention include biguanides (e.g., metformin), glucosidase inhibitors (e.g., acarbose), insulins (including insulin secretagogues or insulin sensitizers), meglitinides (e.g., repaglinide), sulfonylureas (e.g., glimepiride, glyburide and glipizide), biguanide/glyburide combinations (e.g., Glucovance®), thiazolidinediones (e.g., troglitazone, rosiglitazone and pioglitazone), PPAR-alpha agonists, PPAR-gamma agonists, PPAR alpha/gamma dual agonists, SGLT2 inhibitors, glycogen phosphorylase inhibitors, inhibitors of fatty acid binding protein (aP2) such as those disclosed in U.S. Ser. No. 09/519,079 filed Mar. 6, 2000, glucagon-like peptide-1 (GLP-1), and dipeptidyl peptidase IV (DP4) inhibitors.

Examples of suitable anti-osteoporosis agents for use in combination with the compounds of the present invention include alendronate, risedronate, PTH, PTH fragment, raloxifene, calcitonin, steroidal or non-steroidal progesterone receptor agonists, RANK ligand antagonists, calcium sensing receptor antagonists, TRAP inhibitors, selective estrogen receptor modulators (SERM), estrogen, and AP-1 inhibitors.

Examples of suitable anti-obesity agents for use in combination with the compounds of the present invention include aP2 inhibitors, such as those disclosed in U.S. Ser. No. 09/519,079 filed Mar. 6, 2000, PPAR gamma antagonists, PPAR delta agonists, beta 3 adrenergic agonists, such as AJ9677 (Takeda/Dainippon), L750355 (Merck), or CP331648 (Pfizer) or other known beta 3 agonists as disclosed in U.S. Pat. Nos. 5,541,204, 5,770,615, 5,491,134, 5,776,983 and 5,488,064, a lipase inhibitor, such as orlistat or ATL-962 (Alizyme), a serotonin (and dopamine) reuptake inhibitor, such as sibutramine, topiramate (Johnson & Johnson) or axokine (Regeneron), a thyroid receptor beta drug, such as a thyroid receptor ligand as disclosed in WO 97/21993 (U. Cal SF), WO 99/00353 (KaroBio) and GB98/284425 (KaroBio), and/or an anorectic agent, such as dexamphetamine, phentermine, phenylpropanolamine, or mazindol.

Examples of suitable anti-inflammatory agents for use in combination with the compounds of the present invention include prednisone, dexamethasone, Enbrel®, cyclooxygenase inhibitors (i.e., COX-1 and/or COX-2 inhibitors such as NSAIDs, aspirin, indomethacin, ibuprofen, piroxicam, Naproxen®, Celebrex®, Vioxx®), CTLA4-Ig agonists/antagonists, CD40 ligand antagonists, IMPDH inhibitors, such as mycophenolate (CellCept®) integrin antagonists, alpha-4 beta-7 integrin antagonists, cell adhesion inhibitors, interferon gamma antagonists, ICAM-1, tumor necrosis factor (TNF) antagonists (e.g., infliximab, OR1384), prostaglandin synthesis inhibitors, budesonide, clofazimine, CNI-1493, CD4 antagonists (e.g., priliximab), p38 mitogen-activated protein kinase inhibitors, protein tyrosine kinase (PTK) inhibitors, IKK inhibitors, and therapies for the treatment of irritable bowel syndrome (e.g., Zelmac® and Maxi-K® openers such as those disclosed in U.S. Pat. No. 6,184,231 B1).

Examples of suitable anti-anxiety agents for use in combination with the compounds of the present invention include diazepam, lorazepam, buspirone, oxazepam, and hydroxyzine pamoate.

Examples of suitable anti-depressants for use in combination with the compounds of the present invention include citalopram, fluoxetine, nefazodone, sertraline, and paroxetine.

Examples of suitable anti-hypertensive agents for use in combination with the compounds of the present invention include beta adrenergic blockers, calcium channel blockers (L-type and T-type; e.g. diltiazem, verapamil, nifedipine, amlodipine and mybefradil), diuretics (e.g., chlorothiazide, hydrochlorothiazide, flumethiazide, hydroflumethiazide, bendroflumethiazide, methylchlorothiazide, trichloromethiazide, polythiazide, benzthiazide, ethacrynic acid tricrynafen, chlorthalidone, furosemide, musolimine, bumetanide, triamtrenene, amiloride, spironolactone), renin inhibitors, ACE inhibitors (e.g., captopril, zofenopril, fosinopril, enalapril, ceranopril, cilazopril, delapril, pentopril, quinapril, ramipril, lisinopril), AT-1 receptor antagonists (e.g., losartan, irbesartan, valsartan), ET receptor antagonists (e.g., sitaxsentan, atrsentan and compounds disclosed in U.S. Pat. Nos. 5,612,359 and 6,043,265), Dual ET/AII antagonist (e.g., compounds disclosed in WO 00/01389), neutral endopeptidase (NEP) inhibitors, vasopepsidase inhibitors (dual NEP-ACE inhibitors) (e.g., omapatrilat and gemopatrilat), and nitrates.

Examples of suitable anti-platelet agents for use in combination with the compounds of the present invention include GPlIb/IIIa blockers (e.g., abciximab, eptifibatide, tirofiban), P2Y12 antagonists (e.g., clopidogrel, ticlopidine, CS-747), thromboxane receptor antagonists (e.g., ifetroban), aspirin, and PDE-III inhibitors (e.g., dipyridamole) with or without aspirin.

Examples of suitable cardiac glycosides for use in combination with the compounds of the present invention include digitalis and ouabain.

Examples of suitable cholesterol/lipid lowering agents for use in combination with the compounds of the present invention include HMG-CoA reductase inhibitors (e.g., pravastatin, lovastatin, atorvastatin, simvastatin, NK-104 (a.k.a. itavastatin, or nisvastatin or nisbastatin) and ZD-4522 (a.k.a. rosuvastatin, or atavastatin or visastatin)), squalene synthetase inhibitors, fibrates, bile acid sequestrants, ACAT inhibitors, MTP inhibitors, lipooxygenase inhibitors, cholesterol absorption inhibitors, and cholesterol ester transfer protein inhibitors (e.g., CP-529414).

Examples of suitable mineralocorticoid receptor antagonists for use in combination with the compounds of the present invention include spironolactone and eplerinone.

Examples of suitable phospodiesterase inhibitors for use in combination with the compounds of the present invention include PDEIII inhibitors such as cilostazol, and PDE V inhibitors such as sildenafil.

Examples of suitable thyroid mimetics for use in combination with the compounds of the present invention include thyrotropin, polythyroid, KB-130015, and dronedarone.

Examples of suitable anabolic agents for use in combination with the compounds of the present invention include testosterone, TRH diethylstilbesterol, estrogens, β-agonists, theophylline, anabolic steroids, dehydroepiandrosterone, enkephalins, E-series prostagladins, retinoic acid and compounds as disclosed in U.S. Pat. No. 3,239,345, e.g., Zeranol®; U.S. Pat. No. 4,036,979, e.g., Sulbenox® or peptides as disclosed in U.S. Pat. No. 4,411,890.

Examples of suitable HIV or AIDS therapies for use in combination with the compounds of the present invention include indinavir sulfate, saquinavir, saquinavir mesylate, ritonavir, lamivudine, zidovudine, lamivudine/zidovudine combinations, zalcitabine, didanosine, stavudine, and megestrol acetate.

Examples of suitable therapies for treatment of Alzheimer's disease and cognitive disorders for use in combination with the compounds of the present invention include donepezil, tacrine, revastigmine, 5HT6, gamma secretase inhibitors, beta secretase inhibitors, SK channel blockers, Maxi-K blockers, and KCNQs blockers.

Examples of suitable therapies for treatment of sleeping disorders for use in combination with the compounds of the present invention include melatonin analogs, melatonin receptor antagonists, ML1B agonists, and GABA/NMDA receptor antagonists.

Examples of suitable anti-proliferative agents for use in combination with the compounds of the present invention include cyclosporin A, paclitaxel, FK 506, and adriamycin.

Examples of suitable anti-tumor agents for use in combination with the compounds of the present invention include paclitaxel, adriamycin, epothilones, cisplatin, and carboplatin.

Compounds of the present invention may further be used in combination with nutritional supplements such as those described in U.S. Pat. No. 5,179,080, especially in combination with whey protein or casein, amino acids (such as leucine, branched amino acids and hydroxymethylbutyrate), triglycerides, vitamins (e.g., A, B6, B12, folate, C, D and E), minerals (e.g., selenium, magnesium, zinc, chromium, calcium and potassium), carnitine, lipoic acid, creatine, and coenzyme Q-10.

In addition, compounds of the present invention may be used in combination with therapeutic agents used in the treatment of sexual dysfunction, including but not limited to PDE5 inhibitors, such as sildenafil or IC-351; with an antiresorptive agent, hormone replacement therapies, vitamin D analogues, calcitonins, elemental calcium and calcium supplements, cathepsin K inhibitors, MMP inhibitors, vitronectin receptor antagonists, Src SH₂ antagonists, vascular−H⁺-ATPase inhibitors, progesterone receptor agonists, ipriflavone, fluoride, RANK antagonists, PTH and its analogues and fragments, Tibolone, HMG-CoA reductase inhibitors, SERM's, p38 inhibitors, prostanoids, 17-beta hydroxysteroid dehydrogenase inhibitors, and Src kinase inhibitors.

Compounds of the present invention may be used in combination with male contraceptives, such as nonoxynol 9 or therapeutic agents for the treatment of hair loss, such as minoxidil and finasteride or chemotherapeutic agents, such as with LHRH agonists.

For their preferred anticancer or antiangiogenic use, the compounds of the present invention may be administered either alone or in combination with other anti-cancer and cytotoxic agents and treatments useful in the treatment of cancer or other proliferative diseases, for example, where the second drug has the same or different mechanism of action than the present compounds of formula I. Examples of classes of anti-cancer and cytotoxic agents useful in combination with the present compounds include but are not limited to: alkylating agents such as nitrogen mustards, alkyl sulfonates, nitrosoureas, ethylenimines, and triazenes; EGFR inhibitors such as small molecule EGFR inhibitors, EGFR antibodies such as C225 (Erbitux); antimetabolites such as folate antagonists, purine analogues, and pyrimidine analogues; antibiotics such as anthracyclines, bleomycins, mitomycin, dactinomycin, and plicamycin; enzymes such as L-asparaginase; farnesyl-protein transferase inhibitors; 5α reductase inhibitors; inhibitors of 17β-hydroxy steroid dehydrogenase type 3 or type 1; hormonal agents such as glucocorticoids, estrogens/antiestrogens, androgens/antiandrogens, progestins, and luteinizing hormone-releasing hormone antagonists, octreotide acetate; microtubule-disruptor agents, such as ecteinascidins or their analogs and derivatives; microtubule-stabilizing agents such as taxanes, for example, paclitaxel (Taxol®), docetaxel (Taxotere®), and their analogs, and epothilones, such as epothilones A-F and their analogs; plant-derived products, such as vinca alkaloids, epipodophyllotoxins, taxanes; and topiosomerase inhibitors; prenyl-protein transferase inhibitors; and miscellaneous agents such as hydroxyurea, procarbazine, mitotane, hexamethylmelamine, platinum coordination complexes such as cisplatin and carboplatin; and other agents used as anti-cancer and cytotoxic agents such as biological response modifiers, growth factors; immune modulators and monoclonal antibodies. The compounds of the invention may also be used in conjunction with radiation therapy.

Representative examples of these classes of anti-cancer and cytotoxic agents include but are not limited to mechlorethamine hydrochloride, cyclophosphamide, chlorambucil, melphalan, ifosfamide, busulfan, carmustin, lomustine, semustine, streptozocin, thiotepa, dacarbazine, methotrexate, thioguanine, mercaptopurine, fludarabine, pentastatin, cladribin, cytarabine, fluorouracil, doxorubicin hydrochloride, daunorubicin, idarubicin, bleomycin sulfate, mitomycin C, actinomycin D, safracins, saframycins, quinocarcins, discodermolides, vincristine, vinblastine, vinorelbine tartrate, etoposide, etoposide phosphate, teniposide, paclitaxel, tamoxifen, estramustine, estramustine phosphate sodium, flutamide, buserelin, leuprolide, pteridines, diyneses, levamisole, aflacon, interferon, interleukins, aldesleukin, filgrastim, sargramostim, rituximab, BCG, tretinoin, irinotecan hydrochloride, betamethosone, gemcitabine hydrochloride, altretamine, and topoteca and any analogs or derivatives thereof.

Preferred member of these classes include, but are not limited to, paclitaxel, cisplatin, carboplatin, doxorubicin, carminomycin, daunorubicin, aminopterin, methotrexate, methopterin, mitomycin C, ecteinascidin 743, or porfiromycin, 5-fluorouracil, 6-mercaptopurine, gemcitabine, cytosine arabinoside, podophyllotoxin or podophyllotoxin derivatives such as etoposide, etoposide phosphate or teniposide, melphalan, vinblastine, vincristine, leurosidine, vindesine and leurosine.

Examples of anticancer and other cytotoxic agents include the following: epothilone derivatives as found in German Patent No. 4138042.8; WO 97/19086, WO 98/22461, WO 98/25929, WO 98/38192, WO 99/01124, WO 99/02224, WO 99/02514, WO 99/03848, WO 99/07692, WO 99/27890, WO 99/28324, WO 99/43653, WO 99/54330, WO 99/54318, WO 99/54319, WO 99/65913, WO 99/67252, WO 99/67253 and WO 00/00485; cyclin dependent kinase inhibitors as found in WO 99/24416 (see also U.S. Pat. No. 6,040,321); and prenyl-protein transferase inhibitors as found in WO 97/30992 and WO 98/54966; and agents such as those described generically and specifically in U.S. Pat. No. 6,011,029 (the compounds of which U.S. Patent can be employed together with any NHR modulators (including, but not limited to, those of present invention) such as AR modulators, ER modulators, with LHRH modulators, or with surgical castration, especially in the treatment of cancer).

The combinations of the present invention may also be formulated or co-administered with other therapeutic agents that are selected for their particular usefulness in administering therapies associated with the aforementioned conditions. For example, the compounds of the invention may be formulated with agents to prevent nausea, hypersensitivity and gastric irritation, such as antiemetics, and H₁ and H₂ antihistaminics.

As it pertains to the treatment of cancer, the compounds of this invention are most preferably used alone or in combination with anti-cancer treatments such as radiation therapy and/or with cytostatic and/or cytotoxic agents, such as, but not limited to, DNA interactive agents, such as cisplatin or doxorubicin; inhibitors of farnesyl protein transferase, such as those described in U.S. Pat. No. 6,011,029; topoisomerase II inhibitors, such as etoposide; topoisomerase I inhibitors, such as CPT-11 or topotecan; tubulin stabilizing agents, such as paclitaxel, docetaxel, other taxanes, or epothilones; hormonal agents, such as tamoxifen; thymidilate synthase inhibitors, such as 5-fluorouracil; antimetabolites, such as methoxtrexate; antiangiogenic agents, such as angiostatin, ZD6474, ZD6126 and comberstatin A2; kinase inhibitors, such as her2 specific antibodies, Iressa and CDK inhibitors; histone deacetylase inhibitors, such as CI-994 and MS-27-275. Such compounds may also be combined with agents which suppress the production of circulating testosterone such as LHRH agonists or antagonists or with surgical castration. Exemplary combination therapies (e.g., for the treatment of prostate cancer) for use with a compound of the present invention include an LHRH modulator or prednisone.

The present invention also contemplates kits, for example, for the treatment of prostate cancer, comprising a first container (such as a vial) containing a pharmaceutical formulation comprising a compound of the present invention, said compound optionally in a pharmaceutically acceptable carrier, and a second container (such as a vial) containing a pharmaceutical formulation comprising one or more agents (such as an LHRH modulator) to be used in combination with said compound of the present invention, said agent(s) optionally in a pharmaceutically acceptable carrier.

For example, known therapies for advanced metastatic prostate cancer include “complete androgen ablation therapy” wherein tumor growth is inhibited by controlling the supply of androgen to the prostate tissues via chemical castration (castration serves to inhibit the production of circulating testosterone (T) and dihydrotestosterone (DHT)) followed by the administration of androgen receptor (AR) antagonists (which inhibit the function T/DHT derived from the conversion of circulating androgen precursors to T/DHT by the prostate tissue). The compounds of the present invention may be employed as AR antagonists in complete ablation therapy, alone or in combination with other AR antagonists such as Flutamide, Casodex, Nilutamide, or Cyproterone acetate.

The present invention also contemplates use of an antiestrogen and/or aromatase inhibitor in combination with a compound of the present invention, for example, to assist in mitigating side effects associated with antiandrogen therapy such as gynecomastia. Exemplary antiestrogen and/or aromatase inhibitors include anastrozole (Arimidex), tamoxifen citrate (Nolvadex), exemestane (Aromasin), toremifene citrate (Fareston), letrozole (Femara), raloxifene hydrochloride (Evista), Faslodex, or 923 (Wyeth Ayerst).

The compounds of the present invention may be employed adjuvant to surgery.

Another application of the present compounds is in combination with antibody therapy such as but not limited to antibody therapy against PSCA. An additional application is in concert with vaccine/immune modulating agents for the treatment of cancer.

Compounds of the present invention may be employed in accordance with the methods described in U.S. Provisional Patent Application Ser. No. 60/284,438, entitled “Selective Androgen Receptor Modulators and Methods for Their Identification, Design and Use” filed Apr. 18, 2001 by Mark E. Salvati et al., which Provisional Patent Application is incorporated herein by reference in its entirety (including, but not limited to, reference to all specific compounds within Formula I of the present invention), and U.S. patent application Ser. No. 09/885,827, entitled “Selective Androgen Receptor Modulators and Methods for Their Identification, Design and Use” filed Jun. 20, 2001 by Mark E. Salvati et al., which Patent Application is incorporated herein by reference in its entirety (including, but not limited to, reference to all specific compounds within the present invention).

For racemates of compounds of the present invention, one enantiomer may, for example be a full AR antagonist while the other may be an AR antagonist in tumor tissue while having no activity or agonist activity in nontumor tissue containing the androgen receptor.

The above other therapeutic agents, when employed in combination with the compounds of the present invention, may be used, for example, in those amounts indicated in the Physicians' Desk Reference (PDR) or as otherwise determined by one of ordinary skill in the art.

The following assays may be employed in ascertaining the activity of a compound as a NHR modulator. Preferred are those compounds with an activity greater than about 20 μm for binding or transactivation in any of these assays. Various compounds of the present invention were determined to have AR modulator activity utilizing the transactivation assay, and standard AR binding assays as described following.

### Transactivation Assays

AR Specific Assay

Compounds of the present invention are tested in transactivation assays of a transfected reporter construct and using the endogenous androgen receptor of the host cells. The transactivation assay provides a method for identifying functional agonists and partial agonists that mimic, or antagonists that inhibit, the effect of native hormones, in this case, dihydrotestosterone (DHT). This assay may be used to predict in vivo activity as there is a good correlation in both series of data. See, e.g. T. Berger et al., J. Steroid Biochem. Molec. Biol. 773 (1992), the disclosure of which is herein incorporated by reference.

For the transactivation assay a reporter plasmid is introduced by transfection (a procedure to induce cells to take foreign genes) into the respective cells. This reporter plasmid, comprising the cDNA for a reporter protein, such as secreted alkaline phosphatase (SEAP), controlled by prostate specific antigen (PSA) upstream sequences containing androgen response elements (AREs). This reporter plasmid functions as a reporter for the transcription-modulating activity of AR. Thus, the reporter acts as a surrogate for the products (mRNA then protein) normally expressed by a gene under control of AR and its native hormone. In order to detect antagonists, the transactivation assay is carried out in the presence of constant concentration of the natural AR hormone (DHT) known to induce a defined reporter signal. Increasing concentrations of a suspected antagonist will decrease the reporter signal (e.g., SEAP production). On the other hand, exposing the transfected cells to increasing concentrations of a suspected agonist will increase the production of the reporter signal.

For this assay, LNCaP and MDA 453 cells are obtained from the American Type Culture Collection (Rockville, Md.), and maintained in RPMI 1640 or DMEM medium supplemented with 10% fetal bovine serum (FBS; Gibco) respectively. The respective cells are transiently transfected by electroporation according to the optimized procedure described by Heiser, 130 Methods Mol. Biol., 117 (2000), with the pSEAP2/PSA540/Enhancer reporter plasmid. The reporter plasmid, is constructed as follows: commercial human placental genomic DNA is used to generate by Polymerase Cycle Reaction (PCR) a fragment containing the BglII site (position 5284) and the Hind III site at position 5831 of the human prostate specific antigen promoter (Accession #U37672), Schuur, et al., J. Biol. Chem., 271 (12): 7043-51 (1996). This fragment is subcloned into the pSEAP2/basic (Clontech) previously digested with BglII and HindIII to generate the pSEAP2/PSA540 construct. Then a fragment bearing the fragment of human PSA upstream sequence between positions −5322 and −3873 is amplified by PCR from human placental genomic DNA. A XhoI and a BglII sites are introduced with the primers. The resulting fragment is subcloned into pSEAP2/PSA540 digested with XhoI and BglII respectively, to generate the pSEAP2/PSA540/Enhancer construct. LNCaP and MDA 453 cells are collected in media containing 10% charcoal stripped FBS. Each cell suspension is distributed into two Gene Pulser Cuvetts (Bio-Rad) which then receives 8 μg of the reporter construct, and electoporated using a Bio-Rad Gene Pulser at 210 volts and 960 μFaraday. Following the transfections the cells are washed and incubated with media containing charcoal stripped fetal bovine serum in the absence (blank) or presence (control) of 1 nM dihydrotestosterone (DHT; Sigma Chemical) and in the presence or absence of the standard anti-androgen bicalutamide or compounds of the present invention in concentrations ranging from 10⁻¹⁰ to 10⁻⁵ M (sample). Duplicates are used for each sample. The compound dilutions are performed on a Biomek 2000 laboratory workstation.

After 48 hours, a fraction of the supernatant is assayed for SEAP activity using the Phospha-Light Chemiluminescent Reporter Gene Assay System (Tropix, Inc). Viability of the remaining cells is determined using the CellTiter 96 Aqueous Non-Radioactive Cell Proliferation Assay (MTS Assay, Promega). Briefly, a mix of a tetrazolium compound (3-(4,5-dimethylthiazol-2-yl)-5-(3-carboxymethoxyphenyl)-2-(4-sulfophenyl)-2H-tetrazolium, inner salt; MTS) and an electron coupling reagent (phenazine methosulfate; PMS) are added to the cells. MTS (Owen's reagent) is bioreduced by cells into a formazan that is soluble in tissue culture medium, and therefore its absorbance at 490 nm can be measured directly from 96 well assay plates without additional processing. The quantity of formazan product as measured by the amount of 490 nm absorbance is directly proportional to the number of living cells in culture. For each replicate the SEAP reading was normalized by the Abs490 value derived from the MTS assay. For the antagonist mode, the % Inhibition was calculated as:
% Inhibition=100×(1−[(average control−average blank)/(average sample−average blank)])

Data is plotted and the concentration of compound that inhibited 50% of the normalized SEAP is quantified (IC₅₀).

For the agonist mode % Control is referred as the effect of the tested compound compared to the maximal effect observed with the natural hormone, in this case DHT, and is calculated as:
% Control=100×[(average sample−average blank)/(average control −average blank)]

Data is plotted and the concentration of compound that activates to levels 50% of the normalized SEAP for the control is quantified (EC₅₀).

GR Specificity Assay

The reporter plasmid utilized is comprised of the cDNA for the reporter SEAP protein, as described for the AR specific-transactivation assay. Expression of the reporter SEAP protein is controlled by the mouse mammary tumor virus long terminal repeat (MMTV LTR) sequences that contains three hormone response elements (HREs) that can be regulated by both GR and PR see, e.g. G. Chalepakis et al., Cell, 53(3), 371 (1988). This plasmid is transfected into A549 cells, which expresses endogenous GR, to obtain a GR specific transactivation assay. A549 cells are obtained from the American Type Culture Collection (Rockville, Md.), and maintained in RPMI 1640 supplemented with 10% fetal bovine serum (FBS; Gibco). Determination of the GR specific antagonist activity of the compounds of the present invention is identical to that described for the AR specific-transactivation assay, except that the DHT was replaced with 5 nM dexamethasone (Sigma Chemicals), a specific agonist for GR. Determination of the GR specific agonist activity of the compounds of the present invention is performed as described for the AR transactivation assay, wherein one measures the activation of the GR specific reporter system by the addition of a test compound, in the absence of a known GR specific agonists ligand.

PR Specific Assay

The reporter plasmid utilized is comprised of the cDNA for the reporter SEAP protein, as described for the AR specific-transactivation assay. Expression of the reporter SEAP protein is controlled by the mouse mammary tumor virus long terminal repeat (MMTV LTR) sequences that contains three hormone response elements (HREs) that can be regulated by both GR and PR. This plasmid is transfected into T47D, which expresses endogenous PR, to obtain a PR specific transactivation assay. T47D cells are obtained from the American Type Culture Collection (Rockville, Md.), and maintained in DMEM medium supplemented with 10% fetal bovine serum (FBS; Gibco). Determination of the PR specific antagonist activity of the compounds of the present invention is identical to that described for the AR specific transactivation assay, except that the DHT is replaced with 1 nM Promegastone (NEN), a specific agonist for PR. Determination of the PR specific agonist activity of the compounds of the present invention is performed as described for the AR transactivation assay, wherein one measures the activation of the PR specific reporter system by the addition of a test compound, in the absence of a known PR specific agonists ligand.

AR Binding Assay

For the whole cell binding assay, human LNCaP cells (T877A mutant AR) or MDA 453 (wild type AR) in 96-well microtiter plates containing RPMI 1640 or DMEM supplemented with 10% charcoal stripped CA-FBS (Cocaleco Biologicals) respectively, are incubated at 37° C. to remove any endogenous ligand that might be complexed with the receptor in the cells. After 48 hours, either a saturation analysis to determine the K_{d} for tritiated dihydrotestosterone, [³H]-DHT, or a competitive binding assay to evaluate the ability of test compounds to compete with [³H]-DHT is performed. For the saturation analysis, media (RPMI 1640 or DMEM −0.2% CA-FBS) containing [³H]-DHT (in concentrations ranging from 0.1 nM to 16 nM) in the absence (total binding) or presence (non-specific binding) of a 500-fold molar excess of unlabeled DHT is added to the cells. After 4 hours at 37° C., an aliquot of the total binding media at each concentration of [³H]-DHT is removed to estimate the amount of free [³H]-DHT. The remaining media is removed, cells are washed three times with PBS and harvested onto UniFilter GF/B plates (Packard), Microscint (Packard) is added and plates are counted in a Top-Counter (Packard) to evaluate the amount of bound [³H]-DHT.

For the saturation analysis, the difference between the total binding and the non-specific binding, is defined as specific binding. The specific binding is evaluated by Scatchard analysis to determine the K_{d} for [³H]-DHT. See e.g. D. Rodbard, Mathematics and statistics of ligand assays: an illustrated guide: In: J. Langon and J. J. Clapp, eds., Ligand Assay, Masson Publishing U.S.A., Inc., New York, pp. 45-99, (1981), the disclosure of which is herein incorporated by reference.

For the competition studies, media containing 1 nM [³H]-DHT and compounds of the invention (“test compounds”) in concentrations ranging from 10⁻¹⁰ to 10⁻⁵ M are added to the cells. Two replicates are used for each sample. After 4 hours at 37° C., cells are washed, harvested, and counted as described above. The data is plotted as the amount of [³H]-DHT (% of control in the absence of test compound) remaining over the range of the dose response curve for a given compound. The concentration of test compound that inhibited 50% of the amount of [³H]-DHT bound in the absence of competing ligand is quantified (IC₅₀) after log-logit transformation. The K₁ values are determined by application of the Cheng-Prusoff equation to the IC₅₀ values, where:

$${K}_{I} = \frac{{IC}_{50}}{( 1 + ( {}^{3} H \text{-} DHT ) / {K}_{d} for {}^{3} H \text{-} DHT )}$$

After correcting for non-specific binding, IC₅₀ values are determined. The IC₅₀ is defined as the concentration of competing ligand needed to reduce specific binding by 50%. The K_{d}s for [³H]-DHT for MDA 453 and LNCaP are 0.7 and 0.2 nM respectively.

Human Prostate Cell Proliferation Assay

Compounds of the present invention are tested (“test compounds”) on the proliferation of human prostate cancer cell lines. For that, MDA PCa2b cells, a cell line derived from the metastasis of a patient that failed castration, Navone et al., Clin. Cancer Res., 3, 2493-500 (1997), are incubated with or without the test compounds for 72 hours and the amount of [³H]-thymidine incorporated into DNA is quantified as a way to assess number of cells and therefore proliferation. The MDA PCa2b cell line is maintained in BRFF-HPC1 media (Biological Research Faculty & Facility Inc., MD) supplemented with 10% FBS. For the assay, cells are plated in Biocoated 96-well microplates and incubated at 37° C. in 10% FBS (charcoal-stripped)/BRFF-BMZERO (without androgens). After 24 hours, the cells are treated in the absence (blank) or presence of 1 nM DHT (control) or with test compounds (sample) of the present invention in concentrations ranging from 10⁻¹⁰ to 10⁻⁵ M. Duplicates are used for each sample. The compound dilutions are performed on a Biomek 2000 laboratory work station. Seventy two hours later 0.44 uCi. of [³H]-Thymidine (Amersham) is added per well and incubated for another 24 h followed by tripsinization, harvesting of the cells onto GF/B filters. Micro-scint PS is added to the filters before counting them on a Beckman TopCount.

The % Inhibition was calculated as:

% Inhibition=100×(1−[(average_{control}−average_{blank})/(averageₛₐₘₚₗₑ−average_{blank})])

Data is plotted and the concentration of compound that inhibited 50% of the [³H]-Thymidine incorporation is quantified (IC₅₀).

C2C12 Mouse Myoblast Transactivation Assay:

Two functional transactivation assays are developed to assess the efficacy of androgen agonists in a muscle cell background using a luciferase reporter. The first assay (ARTA Stable 1) uses a cell line, Stable 1 (clone #72), which stably expresses the full length rat androgen receptor but requires the transient transfection of an enhancer/reporter. This cell line is derived from C2C 12 mouse moyoblast cells. The second assay (ARTA Stable 2) uses a cell line, Stable 2 (clone #133), derived from Stable 1 which stably expresses both rAR and the enhancer/luciferase reporter.

The enhancer/reporter construct used in this system is pGL3/2XDR-1/luciferase. 2XDR-1 was reported to be an AR specific response element in CV-1 cells, Brown et. al. The Journal of Biological Chemisty 272, 8227-8235, (1997). It is developed by random mutagenesis of an AR/GR consensus enhancer sequence.

ARTA Stable 1

1. Stable 1 cells are plated in 96 well format at 6,000 cells/well in high glucose DMEM without phenol red (Gibco BRL, Cat. No.: 21063-029) containing 10% charcoal and dextran treated FBS (HyClone Cat. No.: SH30068.02), 50 mM HEPES Buffer (Gibco BRL, Cat. No.: 15630-080), 1× MEM Na Pyruvate (Gibco BRL, Cat. No.: 11360-070), 0.5× Antibiotic-Antimycotic, and 800 μg/ml Geneticin (Gibco BRL, Cat. No.: 10131-035).

2. 48 hours later, cells are transfected with pGL3/2XDR-1/luciferase using LipofectAMINE Plus™ Reagent (Gibco BRL, Cat. No.: 10964-013). Specifically, 5 ng/well pGL3/2XDR-1/luciferase DNA and 50 ng/well Salmon Sperm DNA (as carrier) are diluted with 5 μl/well Opti-MEMem media (Gibco BRL, Cat. No.: 31985-070). To this, 0.5 μl/well Plus reagent is added. This mixture is incubated for 15 minutes at RT. In a separate vessel, 0.385 μl/well LipofectAMINE reagent is diluted with 5 μl/well Opti-MEM. The DNA mixture is then combined with the LipofectAMINE mixture and incubated for an additional 15 minutes at RT. During this time, the media from the cells is removed and replaced with 60 μl/well of Opti-MEM. To this is added 10 μl/well of the DNA/LipofectAMINE transfection mixture. The cells are incubated for 4 hours.

3. The transfection mixture is removed from the cells and replaced with 90 μl of media as in #1 above.

4. 10 μl/well of appropriate drug dilution is placed in each well.

5. 24 hours later, the Steady-Glo™ Luciferase Assay System is used to detect activity according to the manufacturer's instructions (Promega, Cat. No.: E2520).

ARTA stable 2

1. Stable 2 cells are plated in 96 well format at 6,000 cells/well in high glucose DMEM without phenol red (Gibco BRL, Cat. No.: 21063-029) containing 10% charcoal and dextran treated FBS (HyClone Cat. No.: SH30068.02), 50 mM HEPES Buffer (Gibco BRL, Cat. No.: 15630-080), 1× MEM Na Pyruvate (Gibco BRL, Cat. No.: 11360-070), 0.5× Antibiotic-Antimycotic, 800 μg/ml Geneticin (Gibco BRL, Cat. No.: 10131-035) and 800 μg/ml Hygromycin β (Gibco BRL, Cat. No.: 10687-010).

2. 48 hours later, the media on the cells is removed and replaced with 90 μl fresh. 10 μl/well of appropriate drug dilution is placed in each well.

3. 24 hours later, the Steady-Glo™ Luciferase Assay System is used to detect activity according to the manufacturer's instructions (Promega, Cat. No.: E2520).

See U.S. patent application Ser. No. 09/885,831, entitled “Cell Lines and Cell-Based Assays for Identification of Androgen Receptor Modulators” filed Jun. 20, 2001 by Jacek Ostrowski et al., published as U.S. patent application Publication 2002/0058290, which Patent Application is incorporated herein by reference in its entirety.

### Proliferation Assays

Murine Breast Cell Proliferation Assay

The ability of compounds of the present invention (“test compounds”) to modulate the function of AR is determined by testing said compounds in a proliferation assay using the androgen responsive murine breast cell line derived from the Shionogi tumor, Hiraoka et al., Cancer Res., 47, 6560-6564 (1987). Stable AR dependent clones of the parental Shionogi line are established by passing tumor fragments under the general procedures originally described in Tetuo, et. al., Cancer Research 25, 1168-1175 (1965). From the above procedure, one stable line, SC114, is isolated, characterized, and utilized for the testing of example compounds. SC114 cells are incubated with or without the test compounds for 72 hours and the amount of [3H]-thymidine incorporated into DNA is quantified as a surrogate endpoint to assess the number of cells and therefore the proliferation rate as described in Suzuki et. al., J. Steroid Biochem. Mol. Biol. 37, 559-567 (1990). The SC114 cell line is maintained in MEM containing 10⁻⁸ M testosterone and 2% DCC-treated FCS. For the assay, cells are plated in 96-well microplates in the maintenance media and incubated at 37° C. On the following day, the medium is changed to serum free medium [Ham's F-12:MEM (1:1, v/v) containing 0.1% BSA] with (antagonist mode) or without (agonist mode) 10⁻⁸ M testosterone and the test compounds of the present invention in concentrations ranging from 10⁻¹⁰ to 10⁻⁵ M. Duplicates are used for each sample. The compound dilutions are performed on a Biomek 2000 laboratory work station. Seventy two hours later 0.44 uCi of [3H]-Thymidine (Amersham) is added per well and incubated for another 2 hr followed by tripsinization, and harvesting of the cells onto GF/B filters. Micro-scint PS are added to the filters before counting them on a Beckman TopCount.

For the antagonist mode, the % Inhibition was calculated as:
% Inhibition=100×(1−[(averageₛₐₘₚₗₑ−average_{blank})/(average_{control}−average_{blank})])

Data is plotted and the concentration of compound that inhibited 50% of the [³H]-Thymidine incorporation is quantified (IC₅₀).

For the agonist mode % Control is referred as the effect of the tested compound compared to the maximal effect observed with the natural hormone, in this case DHT, and is calculated as:
% Control=100×(averageₛₐₘₚₗₑ−average_{blank})/(average_{control}−average_{blank})

Data is plotted and the concentration of compound that inhibited 50% of the [³ H]-Thymidine incorporation is quantified (EC₅₀).

In Vitro Assay to Measure GR Induced AP-1 Transrepression

The AP-1 assay is a cell based luciferase reporter assay. A549 cells, which contain endogenous glucocorticoid receptor, are stably transfected with an AP-1 DNA binding site attached to the luciferase gene. Cells are then grown in RPMI+10% fetal calf serum (charcoal-treated)+Penicillin/Streptomycin with 0.5 mg/ml geneticin. Cells are plated the day before the assay at approximately 40000 cells/well. On assay day, the media is removed by aspiration and 20 μl assay buffer (RPMI without phenol red+10% FCS (charcoal-treated)+Pen/Strep) is added to each well. At this point either 20 μl assay buffer (control experiments), the compounds of the present invention (“test compounds”) (dissolved in DMSO and added at varying concentrations) or dexamethasome (100 nM in DMSO, positive control) are added to each well. The plates are then pre-incubated for 15 minutes at 37° C., followed by stimulation of the cells with 10 ng/ml PMA. The plates are then incubated for 7 hrs at 37° C. after which 40 μl luciferase substrate reagent is added to each well. Activity is measured by analysis in a luminometer as compared to control experiments treated with buffer or dexamethasome. Activity is designated as % inhibition of the reporter system as compared to the buffer control with 10 ng/ml PMA alone. The control, dexamethasone, at a concentration of ≦10 μM typically suppresses activity by 65%. Test compounds which demonstrate an inhibition of PMA induction of 50% or greater at a concentration of test compound of ≦10 μM are deemed active.

Wet Prostate Weight Assay AR Antagonist Assay

The activity of compounds of the present invention as AR antagonists is investigated in an immature male rat model, a standard, recognized test of antiandrogen activity of a given compound, as described in L. G. Hershberger et al., Proc. Soc. Expt. Biol. Med., 83, 175 (1953); P. C. Walsh and R. F. Gittes, “Inhibition of extratesticular stimuli to prostate growth in the castrated rat by antiandrogens”, Endocrinology, 86, 624 (1970); and B. J. Furr et al., “ICI 176,334: A novel non-steroid, peripherally selective antiandrogen”, J. Endocrinol., 113, R7-9 (1987), the disclosures of which are herein incorporated by reference.

The basis of this assay is the fact that male sexual accessory organs, such as the prostate and seminal vesicles, play an important role in reproductive function. These glands are stimulated to grow and are maintained in size and secretory function by the continued presence of serum testosterone (T), which is the major serum androgen (>95%) produced by the Leydig cells in the testis under the control of the pituitary luteinizing hormone (LH) and follicle stimulating hormone (FSH). Testosterone is converted to the more active form, dihydrotestosterone, (DHT), within the prostate by 5α-reductase. Adrenal androgens also contribute about 20% of total DHT in the rat prostate, compared to 40% of that in 65-year-old men. F. Labrie et al. Clin. Invest. Med., 16, 475-492 (1993). However, this is not a major pathway, since in both animals and humans, castration leads to almost complete involution of the prostate and seminal vesicles without concomitant adrenalectomy. Therefore, under normal conditions, the adrenals do not support significant growth of prostate tissues. M. C. Luke and D. S. Coffey, “The Physiology of Reproduction” ed. By E. Knobil and J. D. Neill, 1, 1435-1487 (1994). Since the male sex organs are the tissues most responsive to modulation of the androgen activity, this model is used to determine the androgen dependent growth of the sex accessory organs in immature castrated rats.

Male immature rats (19-20 days old Sprague-Dawley, Harlan Sprague-Dawely) are castrated under metofane anesthesia. Five days after surgery these castrated rats (60-70 g, 23-25 day-old) are dosed for 3 days. Animals are dosed sub-cutaneously (s.c.) 1 mg/kg with Testosterone Propionate (TP) in arachis oil vehicle and anti-androgen test compounds (compounds of the present invention) are dosed orally by gavage (p.o.) in dissolved/suspensions of 80% PEG 400 and 20% Tween 80 (PEGTW). Animals are dosed (v/w) at 0.5 ml of vehicle/100 g body weight. Experimental groups are as follows:
1. Control vehicle
2. Testosterone Propionate (TP) (3 mg/rat/day, subcutaneous)
3. TP plus Casodex (administered p.o. in PEGTW, QD), a recognized antiandrogen, as a reference compound.
4. To demonstrate antagonist activity, a compound of the present invention (“test compound”) is administered (p.o. in PEGTW, QD) with TP (s.c. as administered in group 2) in a range of doses.
5. To demonstrate agonist activity a compound of the present invention (“test compound”) is administered alone (p.o. in PEGTW, QD) in a range of doses.

At the end of the 3-day treatment, the animals are sacrificed, and the ventral prostate weighed. To compare data from different experiments, the sexual organs weights are first standardized as mg per 100 g of body weight, and the increase in organ weight induced by TP was considered as the maximum increase (100%). ANOVA followed by one-tailed Student or Fischer's exact test is used for statistical analysis.

The gain and loss of sexual organ weight reflect the changes of the cell number (DNA content) and cell mass (protein content), depending upon the serum androgen concentration. See Y. Okuda et al., J. Urol., 145, 188-191 (1991), the disclosure of which is herein incorporated by reference. Therefore, measurement of organ wet weight is sufficient to indicate the bioactivity of androgens and androgen antagonist. In immature castrated rats, replacement of exogenous androgens increases seminal vesicles (SV) and the ventral prostate (VP) in a dose dependent manner.

The maximum increase in organ weight is 4 to 5-fold when dosing 3 mg/rat/day of testosterone (T) or 1 mg/rat/day of testosterone propionate (TP) for 3 days. The EC₅₀ of T and TP are about 1 mg and 0.03 mg, respectively. The increase in the weight of the VP and SV also correlates with the increase in the serum T and DHT concentration. Although administration of T showed 5-times higher serum concentrations of T and DHT at 2 hours after subcutaneous injection than that of TP, thereafter, these high levels declined very rapidly. In contrast, the serum concentrations of T and DHT in TP-treated animals are fairly consistent during the 24 hours, and therefore, TP showed about 10-30-fold higher potency than free T.

In this immature castrated rat model, a known AR antagonist (Casodex) is also administered simultaneously with 0.1 mg of TP (ED₈₀), inhibiting the testosterone-mediated increase in the weights of the VP and SV in a dose dependent manner. The antagonist effects are similar when dosing orally or subcutaneously. Compounds of the invention also exhibit AR antagonist activity by suppressing the testosterone-mediated increase in the weights of VP and SV.

Levator Ani & Wet Prostate Weight Assay AR Agonist Assay

The activity of compounds of the present invention as AR agonists is investigated in an immature male rat model, a recognized test of anabolic effects in muscle and sustaining effects in sex organs for a given compound, as described in L. G. Hershberger et al., Proc. Soc. Expt. Biol. Med., 83, 175 (1953); B. L. Beyler et al, “Methods for evaluating anabolic and catabolic agents in laboratory animals”, J. Amer. Med. Women's Ass., 23, 708 (1968); H. Fukuda et al., “Investigations of the levator ani muscle as an anabolic steroid assay”, Nago Dai. Yak. Ken. Nem. 14, 84 (1966) the disclosures of which are herein incorporated by reference.

The basis of this assay lies in the well-defined action of androgenic agents on the maintenance and growth of muscle tissues and sexual accessory organs in animals and man. Androgenic steroids, such as testosterone (T), have been well characterized for their ability to maintain muscle mass. Treatment of animals or humans after castrations with an exogenous source of T results in a reversal of muscular atrophy. The effects of T on muscular atrophy in the rat levator ani muscle have been well characterized. M. Masuoka et al., “Constant cell population in normal, testosterone deprived and testosterone stimulated levator ani muscles” Am. J. Anat. 119, 263 (1966); Z. Gori et al., “Testosterone hypertrophy of levator ani muscle of castrated rats. I. Quantitative data” Boll.-Soc. Ital. Biol. Sper. 42, 1596 (1966); Z. Gori et al., “Testosterone hypertrophy of levator ani muscle of castrated rats. II. Electron-microscopic observations” Boll.-Soc. Ital. Biol. Sper. 42, 1600 (1966); A. Boris et al., Steroids 15, 61 (1970). As described above, the effects of androgens on maintenance of male sexual accessory organs, such as the prostate and seminal vesicles, is well described. Castration results in rapid involution and atrophy of the prostate and seminal vesicles. This effect can be reversed by exogenous addition of androgens. Since both the levator ani muscle and the male sex organs are the tissues most responsive to the effects of androgenic agents, this model is used to determine the androgen dependent reversal of atrophy in the levator ani muscle and the sex accessory organs in immature castrated rats. Sexually mature rats (200-250 g, 6-8 weeks-old, Sprague-Dawley, Harlan) were acquired castrated from the vendor (Taconic). The rats were divided into groups and treated daily for 7 to 14 days with one of the following:
1. Control vehicle
2. Testosterone Propionate (TP) (3 mg/rat/day, subcutaneous)
3. TP plus Casodex (administered p.o. in PEGTW, QD), a recognized antiandrogen, as a reference compound.
4. To demonstrate antagonist activity, a compound of the present invention (“test compound”) was administered (p.o. in PEGTW, QD) with TP (s.c. as administered in group 2) in a range of doses.
5. To demonstrate agonist activity a compound of the present invention (“test compound”) was administered alone (p.o. in PEGTW, QD) in a range of doses.

At the end of the 7-14-day treatment, the animals are sacrificed by carbon dioxide, and the levator ani, seminal vesicle, and ventral prostate are weighed. To compare data from different experiments, the levator ani muscle and sexual organ weights are first standardized as mg per 100 g of body weight, and the increase in organ weight induced by TP is considered as the maximum increase (100%). Super-ANOVA (one factor) is used for statistical analysis.

The gain and loss of sexual organ weight reflect the changes of the cell number (DNA content) and cell mass (protein content), depending upon the serum androgen concentration. See Y. Okuda et al., J. Urol., 145, 188-191 (1991), the disclosure of which is herein incorporated by reference. Therefore, measurement of organ wet weight is sufficient to indicate the bioactivity of androgens and androgen antagonist. In immature castrated rats, replacement of exogenous androgens increases levator ani, seminal vesicles (SV) and prostate in a dose dependent manner.

The maximum increase in organ weight is 4 to 5-fold when dosing 3 mg/rat/day of testosterone (T) or 1 mg/rat/day of testosterone propionate (TP) for 3 days. The EC₅₀ of T and TP are about 1 mg and 0.03 mg, respectively. The increase in the weight of the VP and SV also correlates with the increase in the serum T and DHT concentration. Although administration of T shows 5-times higher serum concentrations of T and DHT at 2 hours after subcutaneous injection than that of TP, thereafter, these high levels decline very rapidly. In contrast, the serum concentrations of T and DHT in TP-treated animals are fairly consistent during the 24 hours, and therefore, TP showed about 10-30-fold higher potency than free T.

MDA PCa2b Human Prostate Zenograft Assay

In Vivo Antitumor Testing: MDA-PCa-2b human prostate tumors are maintained in Balb/c nu/nu nude mice. Tumors are propagated as subcutaneous transplants in adult male nude mice (4-6 weeks old) using tumor fragments obtained from donor mice. Tumor passage occurs every 5-6 weeks.

For antitumor efficacy trial, the required number of animals needed to detect a meaningful response are pooled at the start of the experiment and each is given a subcutaneous implant of a tumor fragment (˜50 mg) with a 13-gauge trocar. Tumors are allowed to grow to approx. 100-200 mg (tumors outside the range were excluded) and animals are evenly distributed to various treatment and control groups. Treatment of each animal is based on individual body weight. Treated animals are checked daily for treatment related toxicity/mortality. Each group of animals is weighed before the initiation of treatment (Wt1) and then again following the last treatment dose (Wt2). The difference in body weight (Wt2−Wt1) provides a measure of treatment-related toxicity.

Tumor response is determined by measurement of tumors with a caliper twice a week, until the tumors reach a predetermined “target” size of 0.5 gm. Tumor weights (mg) are estimated from the formula: Tumor weight=(length×width2)÷2

Tumor response end-point is expressed in terms of tumor growth inhibition (% T/C), defined as the ratio of median tumor weights of the treated tumors (T) to that of the control group (C).

To estimate tumor cell kill, the tumor volume doubling time is first calculated with the formula:
TVDT=Median time (days) for control tumors to reach target size−Median time (days) for control tumors to reach half the target size s
And, Log cell kill=(T−C)÷(3.32×TVDT)

Statistical evaluations of data are performed using Gehan's generalized Wilcoxon test.

Dunning Prostate Tumor

Dunning R3327H prostate tumor is a spontaneously derived, well differentiated androgen responsive adenocarcinoma of the prostate (Smolev J K, Heston W D, Scott W W, and Coffey D S, Cancer Treat Rep. 61, 273-287 (1977)). The growth of the R3327H subline is selected for its highly androgen-dependent and reproducible growth in intact male rats. Therefore, this model and other sublines of this tumor have been widely used to evaluate in vivo antitumor activities of antiandrogens such as flutamide and bacilutamide/Casodex (Maucher A., and von Angerer, J. Cancer Res. Clin. Oncol., 119, 669-674 (1993), Furr B. J. A. Euro. URL. 18 (suppl. 3), 2-9 (1990), Shain S. A. and Huot R I. J. Steriod Biochem. 31, 711-718 (1988)).

At the beginning of the study, the Dunning tumor pieces (about 4×4 mm) are transplanted subcutaneously to the flank of mature male Copenhagen rats (6-7 weeks old, Harlan-Sprague Dawley, Indianapolis, Md.). About 6 weeks after the implantation, the animals with tumors of measurable size (about 80-120 mm²) are randomized into treatment groups (8-10 rats/group) and the treatments are initiated. One group of the rats are castrated to serve as the negative control of tumor growth. Animals are treated daily with compounds of the current invention, standard antiandrogens such as bacilutamide or vehicle (control) for an average of 10 to 14 weeks. Test compounds are dissolved in a vehicle of (2.5 ml/kg of body weight) 10% polyethylene glycol and 0.05% Tween-80 in 1% carboxymethyl cellulose, PEG/CMC, (Sigma, St Louis, Mo.). Typical therapeutic experiments would include three groups of three escalating doses for each standard or test compound (in a range of 300-3 mg/kg).

Tumors in the vehicle (control) group reach a size of 1500 to 2500 mm³, whereas the castrated animal group typically shows tumor stasis over the 14 weeks of observation. Animals treated orally with 20 mg/kg of bicalutamide or flutamide would be expected to show a 40% reduction in tumor volumes compared to control after 14 weeks of treatment. The size of tumors are measured weekly by vernier caliper (Froboz, Switzerland), taking perpendicular measurements of length and width. Tumor volumes are measured in mm using the formula: Length×Width×Height=Volume. Statistical differences between treatment groups and control are evaluated using multiple ANOVA analysis followed by one tail non-parametric Student t test.

Mature Rat Prostate Weight Assay

The activity of compounds of the present invention are investigated in a mature male rat model, which is a variation of the Levator ani & wet prostate weight assay described above. The above in vivo assays are recognized assays for determining the anabolic effects in muscle and sustaining effects in sex organs for a given compound, as described in L. G. Hershberger et al., 83 Proc. Soc. Expt. Biol. Med., 175 (1953); B. L. Beyler et al, “Methods for evaluating anabolic and catabolic agents in laboratory animals”, 23 J. Amer. Med. Women's Ass., 708 (1968); H. Fukuda et al., “Investigations of the levator ani muscle as an anabolic steroid assay”, 14 Nago Dai. Yak. Ken. Nem. 84 (1966) the disclosures of which are herein incorporated by reference. The basis of this assay lies in the well-defined action of androgenic agents on the maintenance and growth of muscle tissues and sexual accessory organs in animals and man.

The male sexual accessory organs, such as the prostate and seminal vesicles, play an important role in reproductive function. These glands are stimulated to grow and are maintained in size and secretory function by the continued presence of serum testosterone (T), which is the major serum androgen (>95%) produced by the Leydig cells in the testis under the control of the pituitary luteinizing hormone (LH) and follicle stimulating hormone (FSH). Testosterone is converted to the more active form, dihydrotestosterone, (DHT), within the prostate by 5α-reductase. Adrenal androgens also contribute about 20% of total DHT in the rat prostate, compared to 40% of that in 65-year-old men. F. Labrie et. al. 16 Clin. Invest. Med., 475-492 (1993). However, this is not a major pathway, since in both animals and humans, castration leads to almost complete involution of the prostate and seminal vesicles without concomitant adrenalectomy. Therefore, under normal conditions, the adrenals do not support significant growth of prostate tissues, M. C. Luke and D. S. Coffey, “The Physiology of Reproduction” ed. By E. Knobil and J. D. Neill, 1, 1435-1487 (1994). Since the male sex organs and the levator ani are the tissues most responsive to modulation of the androgen activity, this model is used to determine the activity of compounds that modulate the androgen receptor pathway in mature rats.

Along with its mitogenic activity on tissues such as prostate, seminal vesicle and muscle, testosterone also serves as a negative regulator for its own biosynthesis. Testosterone production in the Leydig cells of the testis is controlled by the level of circulating LH released from the pituitary gland. LH levels are themselves controlled by the level of LHRH produced in the hypothalmic region. Testosterone levels in the blood serve to inhibit the secretion of LHRH and subsequently reduce levels of LH and ultimately the levels of circulating testosterone levels. By measuring blood levels of LH as they are effected by compounds of the present invention (“test compounds”), it is possible to determine the level of agonist or antagonist activity of said compounds at the hypothalamic axis of this endocrine cycle.

Matched sets of Harlan Sprague-Dawely rats (40-42 days old, 180-220 g), are dosed orally by gavage (p.o.) with the test compounds in dissolved/suspensions of 80% PEG 400 and 20% Tween 20 (PEGTW) for 14 days. Two control groups, one intact and one castrated are dose orally only with the PEGTW vehicle. Animals are dosed (v/w) at 0.5 ml of vehicle/100 g body weight. Experimental groups are as follows:
1. Intact vehicle (p.o., PEGTW, QD)
2. Control vehicle (p.o., PEGTW, QD)
3. Bicalutamide (Casodex, a recognized antiandrogen, as a reference compound) or a compound of the present invention, p.o. in PEGTW QD. (in a range of doses).

At the end of the 14-day treatment, the animals are sacrificed, and the ventral prostate, the seminal vesicles, and the levator ani are removed surgically and weighed. To compare data from different experiments, the organs weights are first standardized as mg per 100 g of body weight, and expressed as a percentage of the value of the respective organ in the intact group.

Rat luteinizing hormone (rLH) is quantitatively determined with the Biotrak [125 I] kit (Amersham Pharmacia Biotek), following the manufacturer directions. The assay is based on the competition by the LH present in the serum of the binding of [¹²⁵I] rLH to an Amerlex-M bead/antibody suspension. The radioactivity that remains after incubation with the serum and subsequent washes is extrapolated into a standard curve to obtain a reading in ng/ml.

The gain and loss of sexual organ and levator ani weight reflect the changes of the cell number (DNA content) and cell mass (protein content), depending upon the serum androgen concentration, see Y. Okuda et al., J. Urol., 145, 188-191 (1991), the disclosure of which in herein incorporated by reference. Therefore, measurement of organ wet weight is sufficient to indicate the bioactivity of androgens and androgen antagonist. In the mature rats assay, active agonist agents will have no effect or will increase the weight of one or more of the androgen responsive organs (levator ani, prostate, seminal vessicle) and will have no effect or a suppressive effect on LH secretion. Compounds with antagonist activity will decrease the weight of one or more of the androgen responsive organs (levator ani, prostate, seminal vesicle) and will have no effect or a reduced suppressive effect on LH secretion.

CWR22 Human Prostate Zenograft Assay

In Vivo Antitumor Testing: CWR22 human prostate tumors are maintained in Balb/c nu/nu nude mice. Tumors are propagated as subcutaneous transplants in adult male nude mice (4-6 weeks old) using tumor fragments obtained from donor mice. Tumor passage occurs every 5-6 weeks.

For antitumor efficacy trial, the required number of animals needed to detect a meaningful response are pooled at the start of the experiment and each is given a subcutaneous implant of a tumor fragment (˜50 mg) with a 13-gauge trocar. Tumors are allowed to grow to approx. 100-200 mg (tumors outside the range were excluded) and animals are evenly distributed to various treatment and control groups. Treatment of each animal is based on individual body weight. Treated animals are checked daily for treatment related toxicity/mortality. Each group of animals is weighed before the initiation of treatment (Wt1) and then again following the last treatment dose (Wt2). The difference in body weight (Wt2−Wt1) provides a measure of treatment-related toxicity.

Tumor response is determined by measurement of tumors with a caliper twice a week, until the tumors reach a predetermined “target” size of 0.5 gm. Tumor weights (mg) are estimated from the formula: Tumor weight=(length×width2)÷2.

Tumor response end-point is expressed in terms of tumor growth inhibition (% T/C), defined as the ratio of median tumor weights of the treated tumors (T) to that of the control group (C).

To estimate tumor cell kill, the tumor volume doubling time is first calculated with the formula:
TVDT=Median time (days) for control tumors to reach target size−Median time (days) for control tumors to reach half the target size
And, Log cell kill=(T−C)÷(3.32×TVDT)

Statistical evaluations of data are performed using Gehan's generalized Wilcoxon test.

The following Examples illustrate embodiments of the present invention, and are not intended to limit the scope of the claims. Within certain Examples, one compound of Formula I is prepared and then employed to further prepare one or more additional compounds of the present invention or salts thereof. Methods employed to prepare one compound of Formula I or salt thereof as described herein can be employed as appropriate to prepare other compounds of the invention.

### Abbreviations

The following abbreviations are used herein:
EtOAc=ethyl acetate
DCM=dichloromethane
DMF=dimethylformamide
Me=methyl
Ret. t.=retention time
TFA=trifluoroacetic acid
THF=tetrahydrofuran
TLC=thin layer chromatography
A=heat
t-Bu=tert-butyl
Pd/C=palladium on activated charcoal
TEA=triethylamine
n-BuLi=n-butyllithium
RT=room temperature
LC=liquid chromatography
EtOH=ethanol
DCE=dichloroethane
DMSO=dimethylsulfoxide
MS(ES)=Electro-Spray Mass Spectrometry
mCPBA=m-chloroperoxybenzoic acid
sat=saturated
AcOH=acetic acid
MeOH=methanol
Ac=acetyl
DEAD=diethyl azodicarboxylate
h=hours
Et=ethyl
TBAF=tetrabutylammonium fluoride
DCC=Dicyclohexylcarbodiimide
Wilkinson's catalyst=RhCl(PPh₃)₃
DMA=dimethylacetamide
DME=1,2-dimethoxyethane
HRMS=high resolution mass spectrometry
Pd₂(dba)₃=palladium dibenzylidene acetone

### EXAMPLE 1

### (2R,S)-4-(8-Cyano-quinolin-5-yl)-2,5-dimethyl-piperazine-1-carboxylic acid (4-trifluoromethyl-pyridin-3-yl)-amide

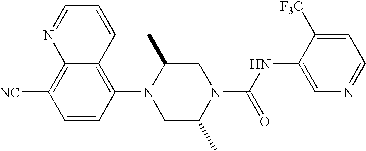

### A. Preparation of (2R)-2-Benzylamino-propionic acid methyl ester (1A)

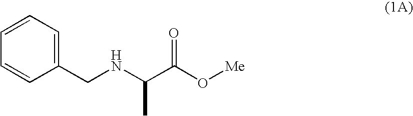

Benzaldehyde (20 ml, 0.2 mol) and TEA (25 ml, 0.18 mol) were added to D-alanine methyl ester hydrochloride (25 g, 0.18 mol) in THF (300 ml) at RT. After 48 hrs, the reaction mixture was filtered through celite (the mixture was washed with 150 ml THF) and concentrated. The reaction crude was dissolved in MeOH (400 ml) and cooled to 0° C. Sodium borohydride (7.5 g, 0.2 mol) was slowly added in portions, and the reaction mixture was stirred at 0° C. for 3 hrs. The reaction was quenched with 1N NaOH (125 ml), concentrated and extracted with DCM (4×200 ml), dried over Na₂SO₄, and concentrated to isolate 1A as a clear, off-white oil (31.7 g, 91%). [M+H]⁺=194.

### B. Preparation of (3S,6R)-1-Benzyl-3,6-dimethyl-piperazine-2,5-dione (1B)

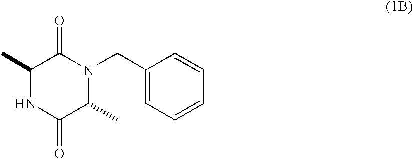

(2R)-2-Benzylamino-propionic acid methyl ester (1A) (1.0 g, 5.2 mmol) and N-tert-butoxycarbonyl-L-alanine (0.98 g, 5.2 mmol) were added to DCC (1.07 g, 5.2 mmol) in DCM (55 ml) at 0° C. After addition, the reaction mixture was warmed to RT, stirred for 24 hrs, filtered through celite (with 2×50 ml diethyl ether wash), and concentrated. The reaction mixture was dissolved in DCM (30 ml), cooled to 0° C., and TFA (5 ml) was added. After 10 min, the reaction mixture was warmed to RT and stirred for 3 hrs. The reaction was quenched by slow addition of saturated NaHCO₃ (100 ml), and extracted with DCM (3×75 ml), dried over Na₂SO₄ and purified by silica gel flash chromatography (EtOAc) to isolate 1B as a clear oil (0.68 g, 57%). [M+H]⁺=233.14.

### C. Preparation of (2S,5R)-1-Benzyl-2,5-dimethyl-piperazine (1C)

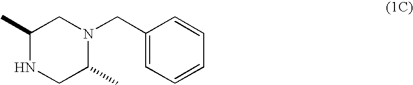

LiAlH₄ (60 mmol, 60 ml of 1.0 M solution in THF) was added to (3S,6R)-1-benzyl-3,6-dimethyl-piperazine-2,5-dione (1B) (3.48 g, 15 mmol) in THF (100 ml) at 0° C. After addition, the reaction mixture was heated at 70° C. for 24 hrs. The reaction was cooled to 0° C. and quenched by slow addition of H₂O (3.5 ml), 1 N NaOH (3.5 ml) and H₂O (3.5 ml). The reaction mixture was filtered through celite and washed with THF (100 ml) and EtOAc (100 ml), dried over Na₂SO₄, concentrated and purified by flash chromatography (15% MeOH/CHCl₃ with 1% TEA) to isolate 1C as a clear oil (2.43 g, 79%). [M+H]⁺=205.16.

### D. Preparation of 5-Bromo-quinoline-8-carbonitrile (1D)

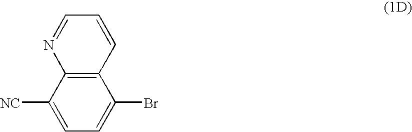

NaNO₂ (345 mg, 5.0 mmol) in H₂O (2.0 ml) was added to 5-amino-quinoline-8-carbonitrile (770 mg, 4.6 mmol) in 48% HBr (aqueous, 2.0 ml) at 0° C. After 30 minutes, CuBr (522 mg, 3.6 mmol) in 48% HBr (aqueous, 1.5 ml) is added. After addition, the reaction mixture was heated at 100° C. for 1 hr and cooled to RT. The reaction was neutralized to pH 8 with 1N NaOH and extracted with EtOAc (2×100 ml). The pooled organic phase was washed with H₂O (100 ml), saturated NH₄OH (100 ml), dried over Na₂SO₄, concentrated, and purified by silica gel flash chromatography (stepwise gradient: DCM to 2% EtOAc/DCM) to isolate compound 1D as a white solid (550 mg, 51%). [M+H]⁺=235.09.

### E. Preparation of (2S,5R)-5-(4-Benzyl-2,5-dimethyl-piperazin-1-yl)-quinoline-8-carbonitrile (1E)

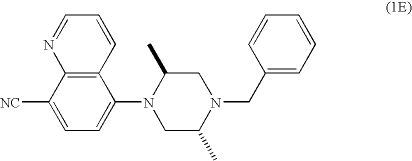

In a microwave compatible reaction flask, (+)-(S)—N,N-Dimethyl-1-[(R)-2-(diphenylphosphino)ferrocenyl]ethylamine (15.4 mg, 0.035 mmol), compound 1D (82 mg, 0.35 mmol), and compound 1C (86 mg, 0.42 mmol) were dissolved in toluene (3.5 ml) and degassed with N₂ for 5 min. Tris(dibenzylideneacetone)dipalladium(0) (32 mg, 0.035 mmol), sodium tert-butoxide (50 mg, 0.52 mmol) were added, and the reaction mixture was degassed with N₂ for 5 additional min. The reaction mixture was heated at 120° C. under high absorption microwave for 40 minutes, diluted with EtOAc (2 ml), filtered, concentrated, and purified using prep HPLC to isolate compound 1E as a TFA salt. Compound 1E was diluted in saturated aqueous NaHCO₃ (10 ml) and extracted with DCM (2×10 ml), and concentrated to isolate 1E as yellow film (17.8 mg, 14%). [M+H]⁺=357.48.

### F. Preparation of (2S,5R)-5-(2,5-Dimethyl-piperazin-1-yl)-quinoline-8-carbonitrile (1F)

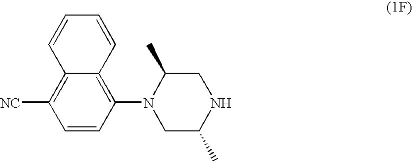

1-Chloroethyl chloroformate (0.054 ml, 0.5 mmol) was added to compound 1E (18 mg, 0.05 mmol) in dichloroethane (1 ml). The reaction mixture was heated at 85° C. for 18 hrs, concentrated and dissolved in MeOH and heated at 65° C. for additional 24 hrs. The reaction mixture was diluted in saturated aqueous NaHCO₃ (10 ml) and extracted with DCM (2×10 ml), dried over Na₂SO₄, concentrated and purified by silica gel flash chromatography (10% MeOH/CHCl₃ with 1% TEA) to isolate compound 1F as yellow film (4.5 mg, 34%). [M+H]⁺=267.36

### G. Preparation of (2R,5S)-4-(8-Cyano-quinolin-5-yl)-2,5-dimethyl-piperazine-1-carboxylic acid(4-trifluoromethyl-pyridin-3-yl)-amide) (1)

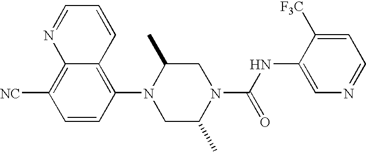

4-Trifluoromethyl-pyridin-3-ylamine (4 mg, 0.025 mmol) and TEA (0.0035 ml, 0.025 mmol) in DCM (0.5 ml) were added to triphosgene (2.5 mg, 0.0085 mmol) in DCM (0.25 ml) at 0° C. After 5 min the reaction mixture was warmed to RT. Compound 1F (4.5 mg, 0.017 mmol) and TEA (0.0035 ml, 0.025 mmol) in DCM (0.5 ml) were added. After 30 minutes, the reaction mixture was concentrated and purified by silica gel flash chromatography (5% MeOH/CHCl₃ with 1% TEA) to isolate Example 1 ((2R,5S)-4-(8-Cyano-quinolin-5-yl)-2,5-dimethyl-piperazine-1-carboxylic acid(4-trifluoromethyl-pyridin-3-yl)-amide) as a clear film (1.2 mg, 16%). HPLC: 92.9% at 1.45 min. (retention time) (Phenomenex S5 ODS column, 4.6×30 mm, eluting with 10-90% aqueous methanol over 2 min containing 0.1% TFA, 5 mL/min, monitoring at 220 nm). MS (ES): m/z 455.37 [M+H]+.

### EXAMPLE 2

Further compounds of the present invention can be prepared by procedures analogous to those described above. Table 1 provides the structures of representative compounds of Formula I that can be prepared using the procedures described in Example 1.

TABLE 1

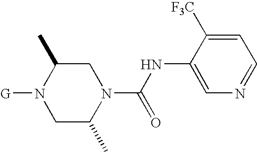

| Example | G = |
|---|---|
| 2-1 | 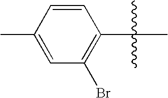 |
| 2-2 | 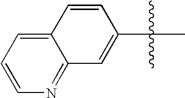 |
| 2-3 | 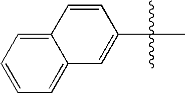 |
| 2-4 | 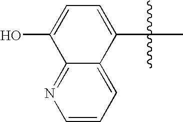 |
| 2-5 | 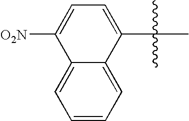 |
| 2-6 | 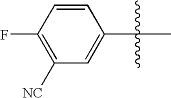 |
| 2-8 | 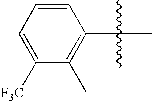 |
| 2-9 | 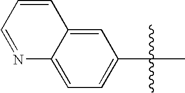 |
| 2-10 | 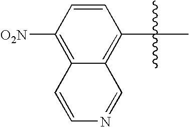 |
| 2-11 | 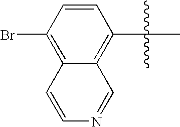 |
| 2-12 | 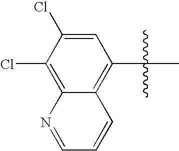 |
| 2-13 | 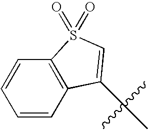 |
| 2-14 | 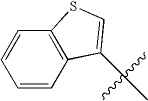 |
| 2-15 | 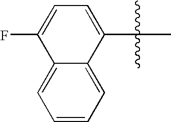 |
| 2-16 | 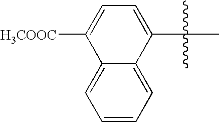 |
| 2-17 | 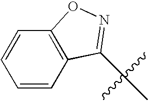 |
| 2-18 | 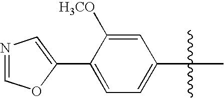 |
| 2-19 | 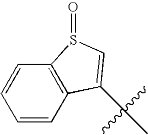 |
| 2-20 | 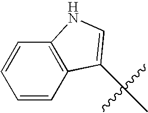 |
| 2-21 | 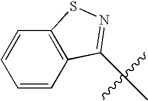 |
| 2-22 | 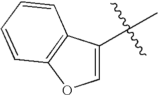 |
| 2-23 | 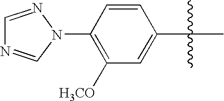 |
| 2-24 | 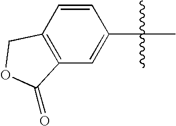 |
| 2-25 | 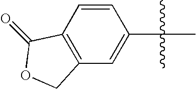 |
| 2-26 | 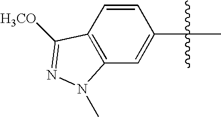 |
| 2-27 | 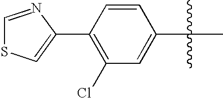 |
| 2-28 | 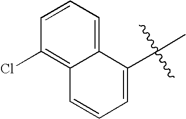 |
| 2-29 | 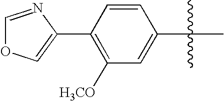 |
| 2-30 | 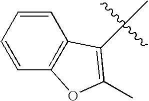 |
| 2-31 | 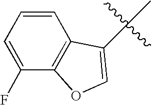 |
| 2-32 | 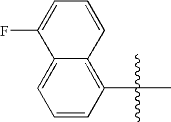 |
| 2-33 | 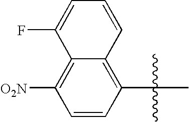 |
| 2-34 | 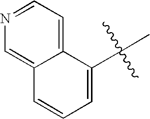 |
| 2-35 | 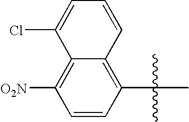 |
| 2-36 | 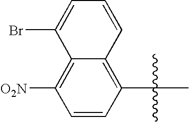 |
| 2-37 | 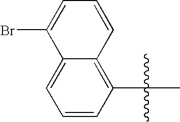 |
| 2-38 | 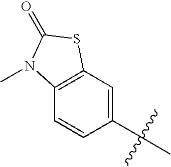 |
| 2-39 | 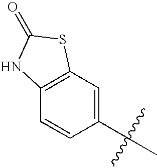 |
| 2-40 | 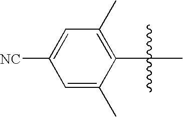 |
| 2-41 | 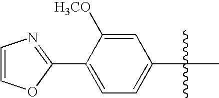 |
| 2-42 | 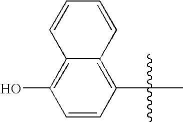 |
| 2-43 | 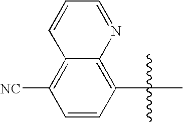 |
| 2-44 | 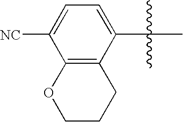 |
| 2-45 | 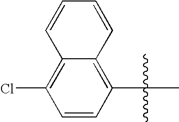 |
| 2-46 | 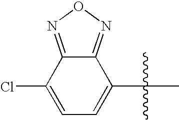 |
| 2-47 | 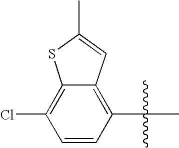 |
| 2-48 | 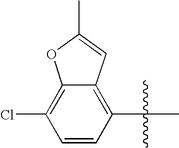 |
| 2-49 | 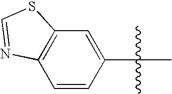 |
| 2-50 | 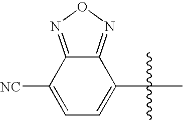 |
| 2-51 | 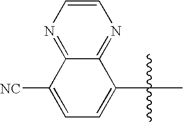 |
| 2-52 | 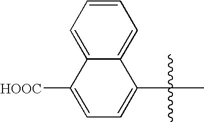 |
| 2-53 | 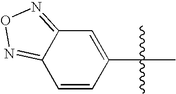 |
| 2-54 | 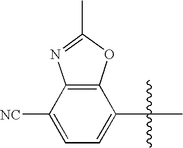 |
| 2-55 | 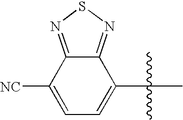 |
| 2-56 | 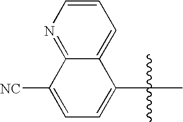 |
| 2-57 | 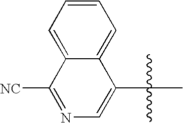 |
| 2-58 | 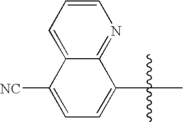 |
| 2-59 | 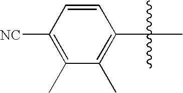 |
| 2-60 | 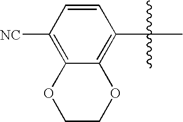 |
| 2-61 | 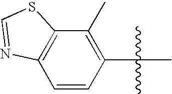 |
| 2-62 | 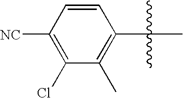 |
| 2-63 | 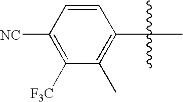 |
| 2-64 | 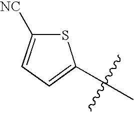 |
| 2-65 | 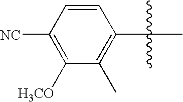 |
| 2-66 | 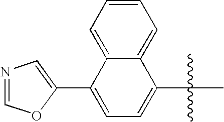 |
| 2-67 | 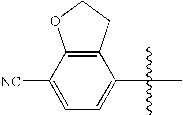 |
| 2-68 | 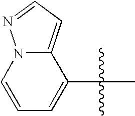 |
| 2-69 | 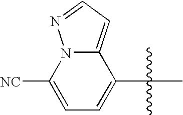 |
| 2-70 | 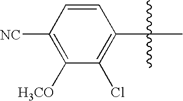 |
| 2-71 | 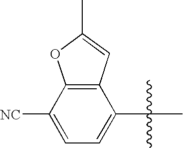 |
| 2-72 | 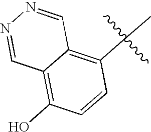 |
| 2-73 | 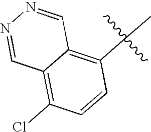 |
| 2-74 | 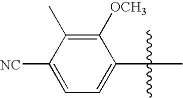 |
| 2-75 | 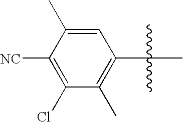 |
| 2-76 | 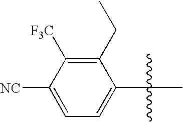 |
| 2-77 | 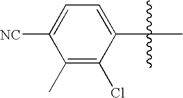 |
| 2-78 | 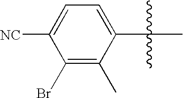 |
| 2-79 | 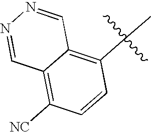 |
| 2-80 | 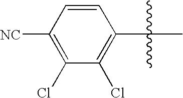 |
| 2-81 | 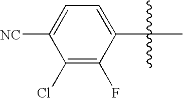 |
| 2-82 | 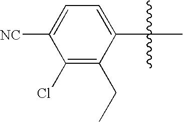 |
| 2-83 | 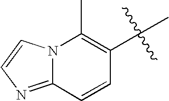 |
| 2-84 | 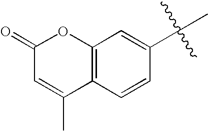 |
| 2-85 | 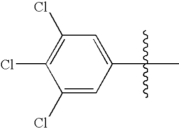 |
| 2-86 | 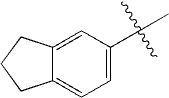 |
| 2-87 | 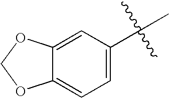 |
| 2-88 | 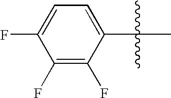 |
| 2-89 | 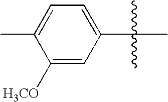 |
| 2-90 | 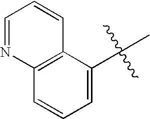 |
| 2-91 | 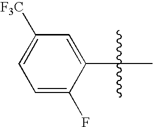 |
| 2-92 | 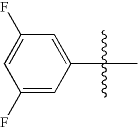 |
| 2-93 | 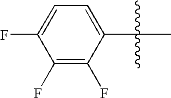 |
| 2-94 | 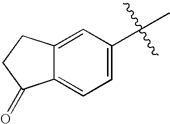 |
| 2-95 | 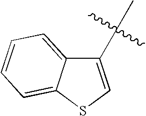 |
| 2-96 | 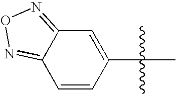 |
| 2-97 | 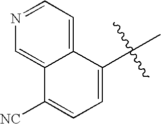 |
| 2-98 | 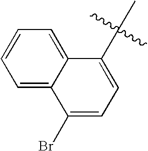 |
| 2-99 | 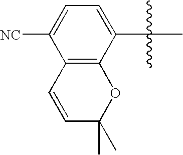 |
| 2-100 | 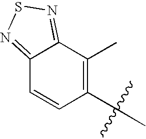 |
| 2-101 | 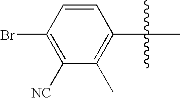 |
| 2-102 | 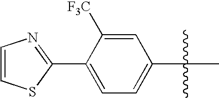 |
| 2-103 | 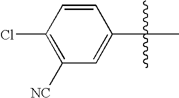 |
| 2-104 | 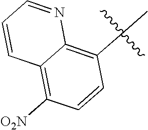 |
| 2-105 | 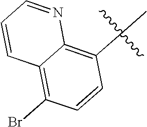 |
| 2-106 | 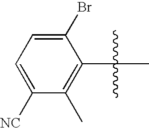 |
| 2-107 | 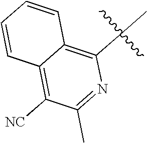 |
| 2-108 | 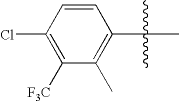 |
| 2-109 | 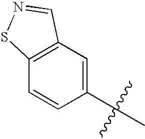 |
| 2-110 | 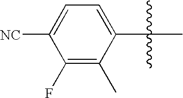 |

### EXAMPLE 3

Further compounds can be prepared by procedures analogous to those described above. Table 2 provides the structures of representative compounds that can be prepared using the procedures described in Example 1.

TABLE 2

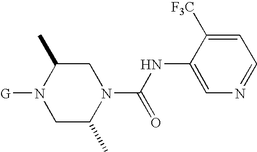

| Example | G = |
|---|---|
| 3-1 | 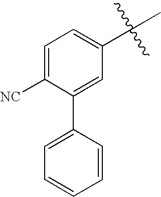 |
| 3-2 | 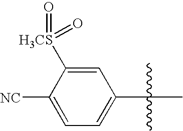 |

When ranges are used herein for physical properties, such as molecular weight, or chemical properties, such as chemical formulae, all combinations and subcombinations of ranges and specific embodiments therein are intended to be included.

The disclosures of each patent, patent application and publication cited or described in this document are hereby incorporated herein by reference, in their entirety.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

1. A method of treating a NHR-associated condition comprising administering to a subject in need of treatment thereof, an effective amount of a compound of Formula (I): 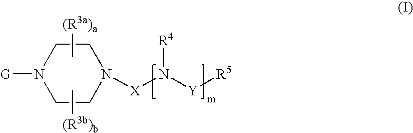
wherein:
G is Ar¹, Ar², or Ar³;
Ar¹ is a bicyclic or tricyclic aryl or heteroaryl optionally substituted with one to four R⁶;
Ar² is a monocyclic five-membered heteroaryl optionally substituted with one to two R⁶; 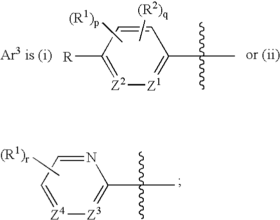
wherein,
Z¹ and Z² are each independently nitrogen or carbon, the carbon atoms of Z¹ and Z² each being bonded to a hydrogen atom or being substituted with a group R¹ or R²;
one of Z³ and Z⁴ is nitrogen and the other of Z³ and Z⁴ is carbon, the carbon atom of Z³ or Z⁴ being bonded to a hydrogen atom or being substituted with a group R¹;
R is R², hydrogen, halogen, haloalkyl, haloalkoxy, alkyl, substituted alkyl, —C(═O)R⁷, —C(═O)—O—R⁷, or —C(═O)NR⁹R¹⁰, and additionally, when either (i) p and q taken together are at least two, and/or (ii) q is at least one, then R may also be cyano or nitro, provided that when Z¹ and Z² are carbon and R is cyano, then the carbon atom of Z¹ is substituted with the group R¹ or R²;
R¹ is alkyl, substituted alkyl, alkenyl, substituted alkenyl, alkynyl, substituted alkynyl, halo, cyano, nitro, cycloalkyl, substituted cycloalkyl, aryl, substituted aryl, heterocyclo, substituted heterocyclo, —OC(═O)R⁷, —C(═O)—O—R⁷, —C(═O)R⁷, —C(═S)R⁷, —C(═O)NR⁹R¹⁰, —CR¹¹R¹²OR⁷, —OR⁷, —NR⁹R¹⁰, —SR⁷, —S(═O)R⁷, —SO₂R⁷, —SO₂OR⁷, and/or —SO₂NR⁷R⁸;
R² is optionally-substituted aryl, cycloalkyl, and/or heterocyclo;
R^{3a} and R^{3b} are each independently alkyl, substituted alkyl, alkenyl, substituted alkenyl, alkynyl, substituted alkynyl, cycloalkyl, substituted cycloalkyl, heterocyclo, substituted heterocyclo, aryl, substituted aryl, cyano, —OR¹³, —C(═O)R¹³, —OC(═O)R¹³, —C(═O)OR¹³, —C(═O)NR¹⁴R¹⁵, —SO₂R¹³, —SO₂OR¹³, —SO₂NR¹⁴R¹⁵, and/or a carbamoyl group which may be substituted by 1 or 2 lower alkyl;
X is —C(═O)—, —C(═S)—, or —SO₂—;
R⁴ is hydrogen, alkyl, substituted alkyl, cyano, —O-lower alkyl, a carbamoyl group which may be substituted by 1 or 2 lower alkyl; a lower alkylene-C(═O)—, or a lower alkylene-O—C(═O)— group;
Y is a bond, lower alkylene, —C(═O)—, or —SO₂—;
R⁵ is alkyl, substituted alkyl, alkenyl, substituted alkenyl, cycloalkyl, substituted cycloalkyl, heterocyclo, substituted heterocyclo, aryl, substituted aryl, —OR⁷, —C(═O)R⁷, —OC(═O)R⁷, —C(═O)OR⁷, or amido which may be substituted by 1 or 2 lower alkyl, aryl, heterocycle or cycloalkyl each of which in turn may optionally be substituted;
alternatively, when m is 1, R⁴ and R⁵ may be linked together to optionally form a five- or six-membered heterocycle;
R⁶ is alkyl, substituted alkyl, alkenyl, substituted alkenyl, alkynyl, substituted alkynyl, halo, cycloalkyl, substituted cycloalkyl, aryl, substituted aryl, heterocyclo, substituted heterocyclo, cyano, nitro, —OC(═O)R⁷, —C(═O)—O—R⁷, —C(═O)R⁷, —C(═S)R⁷, —C(═O)NR⁹R¹⁰, —CR¹¹R¹²OR⁷, —OR⁷, —NR⁹R¹⁰, —SR⁷, —S(═O)R⁷, —SO₂R⁷, —SO₂OR⁷, —SO₂NR⁷R⁸, —P(═O)(OR⁷)(OR⁸), —P(═O)(R⁷)(R⁸), and/or —P(═O)(R⁸)(NHR⁹);
R⁷, R⁸ and R¹³ are each, independently of each other, and at each occurrence, hydrogen, alkyl, substituted alkyl, alkenyl, substituted alkenyl, alkynyl, substituted alkynyl, cycloalkyl, substituted cycloalkyl, heterocyclo, substituted heterocyclo, aryl, and/or substituted aryl, except when R⁷ or R¹³ is attached to a sulfonyl group as in —S(═O)R⁷, —S(═O)R¹³, —SO₂R⁷, —SO₂R¹³, —SO₂OR⁷, and —SO₂OR¹³, then R⁷ and R¹³ are not hydrogen;
R⁹ and R¹⁰ are each independently hydrogen, alkyl, substituted alkyl, alkenyl, substituted alkenyl, alkynyl, substituted alkynyl, cycloalkyl, substituted cycloalkyl, heterocyclo, substituted heterocyclo, aryl, substituted aryl, —C(═O)R⁷, —C(═O)NHR⁷, —SO₂OR⁷, and/or —SO₂NR⁷R⁸;
R¹¹ and R¹² are each independently hydrogen, alkyl, substituted alkyl, alkenyl, substituted alkenyl, alkynyl, substituted alkynyl, cycloalkyl, substituted cycloalkyl, heterocyclo, substituted heterocyclo, aryl, substituted aryl, halo, cyano, hydroxylamine, hydroxamide, alkoxy, substituted alkoxy, —NR⁷R⁸, thiol, alkylthio, and/or substituted alkylthio;
R¹⁴ and R¹⁵ are each independently hydrogen, alkyl, substituted alkyl, cycloalkyl, substituted cycloalkyl, heterocyclo, substituted heterocyclo, aryl, and/or substituted aryl;
one of a and b is 1, and the other of a and b is 0 or 1;
m is 0 or 1;
p and q are 0, 1, and/or 2; and
r is 0, 1, 2, or 3;
or a pharmaceutically acceptable salt, solvate, or N-oxide thereof.

2. The method according to claim 1 in which:
R^{3a} and R^{3b} are each independently lower alkyl and/or substituted lower alkyl;
X is —C(═O)— or —SO₂—;
R⁴ is hydrogen or lower alkyl;
Y is a bond or methylene;
R⁵ is alkyl, substituted alkyl, phenyl, napthyl, cyclohexyl, O-lower alkylene-phenyl, thienyl, furyl, pyridyl, pyrazinyl, pyrimidinyl, ixosazolyl, thiaziazolyl, benzothiazolyl, benzoimidazolyl, tetrazolyl, morpholinyl, tetrahydrofuryl, thiamorpholinyl, benzofurazanyl, or —CO₂(lower alkyl), each of which cyclic groups and alkyl groups of R⁵ may in turn optionally be substituted by one to three groups selected from R¹⁶;
R⁷, R⁸, R⁹, R¹⁰, R¹¹, and R¹² are each independently and at each occurrence hydrogen, lower alkyl, and/or substituted lower alkyl;
R¹⁶ is lower alkyl, halo, nitro, trifluoromethyl, trifluoromethoxy, cyano, alkoxy, phenoxy, alkylthio, hydroxy, carboxy, alkoxycarbonyl, alkylcarbonyloxy, amino, NR¹⁶R¹⁷, carbamoyl, thiol, phenyl, —C(═O)R¹⁷, —C(═O)NR¹⁷R¹⁸, —NR¹⁷SO₂(alkyl), —SO₂NR¹⁷R¹⁸, —S(═O)alkyl, —SO₂alkyl, —O(lower alkylene)-CF₃, phenyl, or morpholinyl; and
R¹⁷ and R¹⁸ are independently hydrogen, alkyl, and/or phenyl;
or a pharmaceutically acceptable salt, solvate, or N-oxide thereof.

3. The method according to claim 1 wherein G is Ar¹.

4. The method according to claim 1 wherein G is Ar².

5. The method according to claim 1, wherein
G is Ar³;
p and q taken together are equal to two or three; and
R is halogen, haloC₁₋₄alkyl, haloC₁₋₄alkoxy, lower alkyl, cyano, or nitro.

6. The method according to claim 1, wherein
G is Ar³;
q is 1; and
R is halogen, haloC₁₋₄alkyl, haloC₁₋₄alkoxy, lower alkyl, cyano, or nitro.

7. The method according to claim 1 wherein G is:

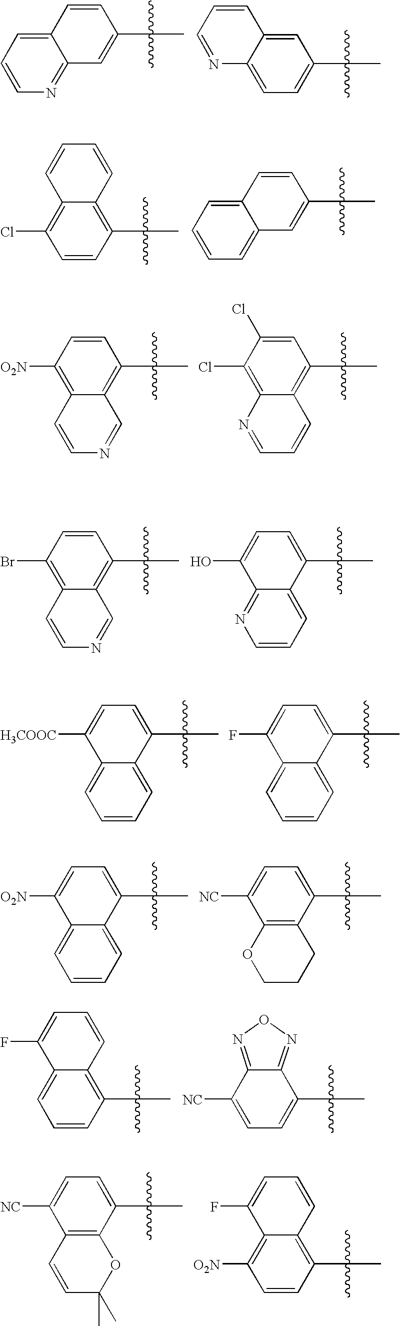

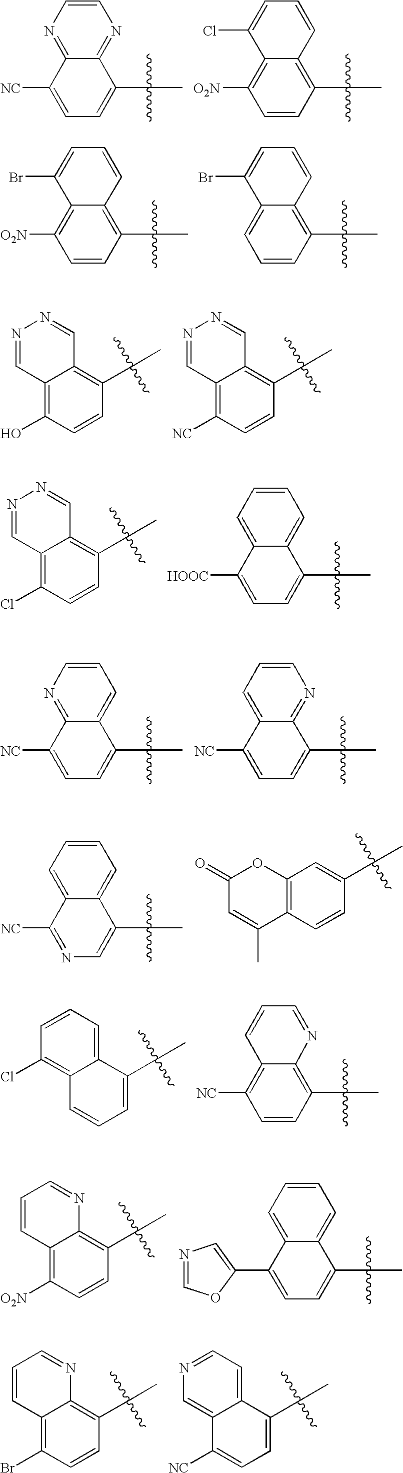

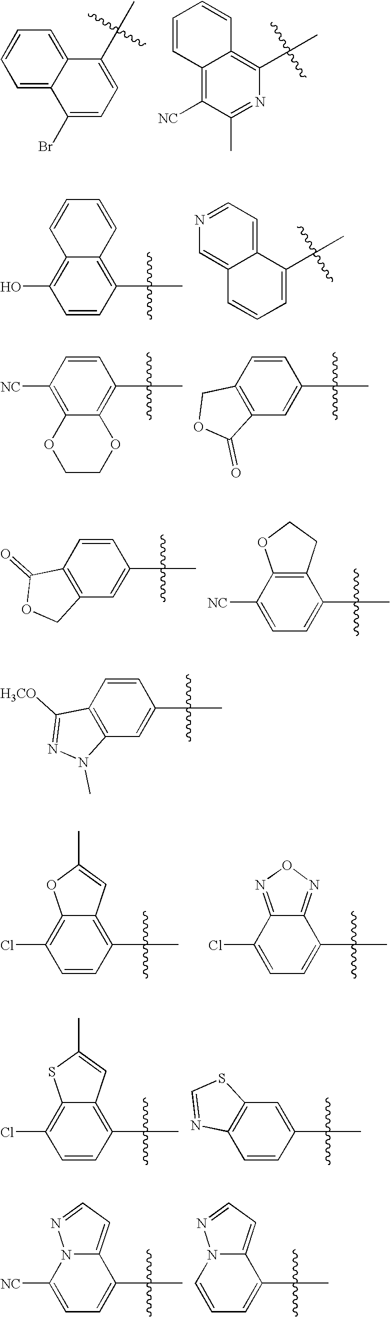

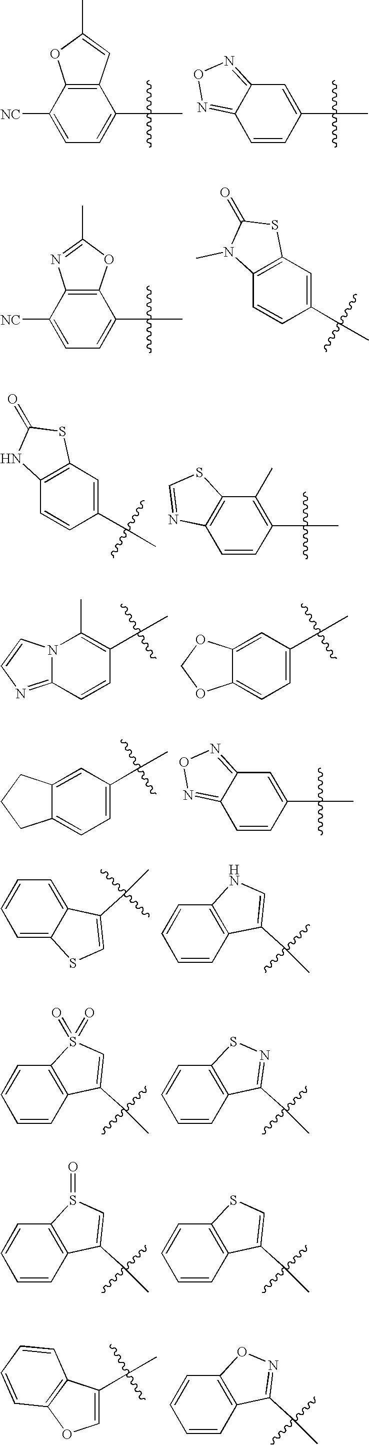

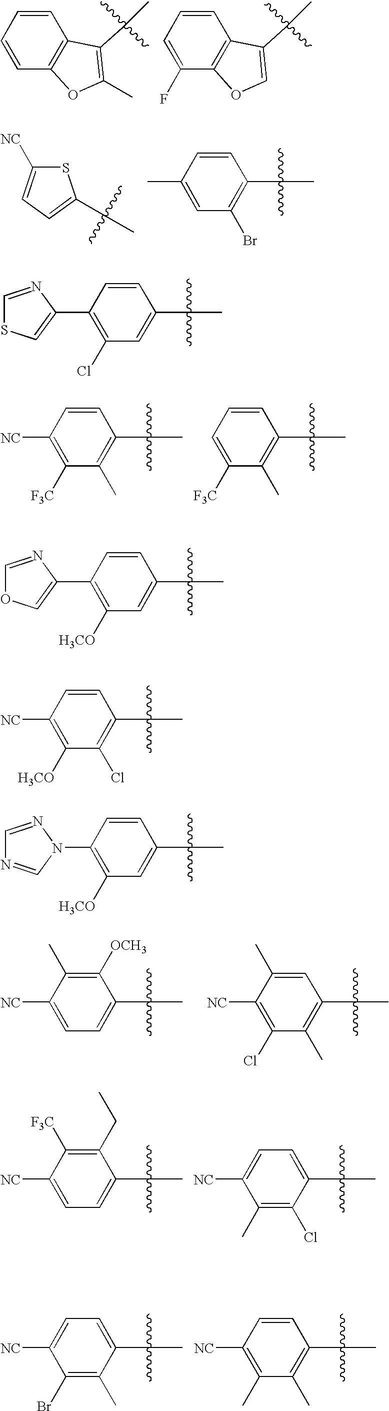

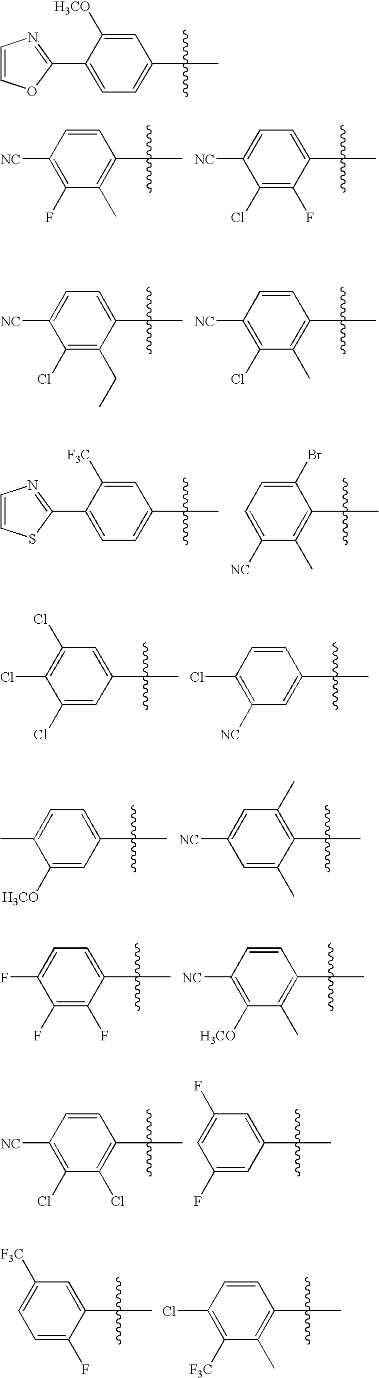

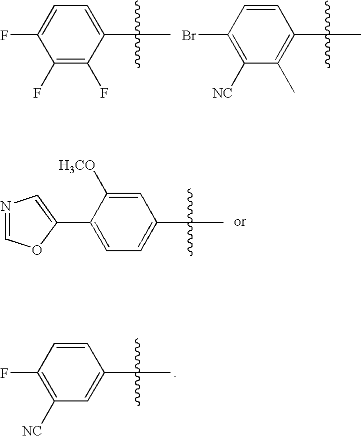

8. A compound having the Formula (I), 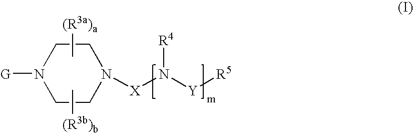
wherein:
G is Ar^{1a}, Ar^{2a}, or Ar^{3a};
Ar^{1a} is a bicyclic or tricyclic aryl or heteroaryl optionally substituted with one to four R⁶, provided, however, that if Ar¹ is 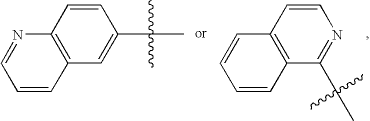
then a and b taken together are at least two;
Ar^{2a} is a monocyclic five-membered heteroaryl optionally substituted with one to two R⁶; 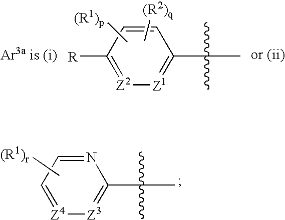
wherein,
Z¹ and Z² are each independently nitrogen or carbon, the carbon atoms of Z¹ and Z² each being bonded to a hydrogen atom or being substituted with a group R¹ or R²;
one of Z³ and Z⁴ is nitrogen and the other of Z³ and Z⁴ is carbon, the carbon atom of Z³ or Z⁴ being bonded to a hydrogen atom or being substituted with a group R¹; provided however, that if Z¹ or Z⁴ is nitrogen, then a and b taken together are at least two;
R is R², hydrogen, halogen, haloalkyl, haloalkoxy, alkyl, substituted alkyl, —C(═O)R⁷, —C(═O)—O—R⁷, or —C(═O)NR⁹R¹⁰, and additionally, when either (i) p and q taken together are at least two, and/or (ii) q is at least one, then R may also be cyano or nitro, provided that if Z¹ and Z² are carbon and R is cyano, then the carbon atom of Z¹ is substituted with the group R¹ or R²;
R¹ is alkyl, substituted alkyl, alkenyl, substituted alkenyl, alkynyl, substituted alkynyl, halo, cyano, nitro, cycloalkyl, substituted cycloalkyl, aryl, substituted aryl, heterocyclo, substituted heterocyclo, —OC(═O)R⁷, —C(═O)—O—R⁷, —C(═O)R⁷, —C(═S)R⁷, —C(═O)NR⁹R¹⁰, —CR¹¹R¹²OR⁷, —OR⁷, —NR⁹R¹⁰, —SR⁷, —S(═O)R⁷, —SO₂R⁷, —SO₂OR⁷, and/or —SO₂NR⁷R⁸;
R² is optionally-substituted aryl, cycloalkyl, and/or heterocyclo;
R^{3a} and R^{3b} are each independently alkyl, substituted alkyl, alkenyl, substituted alkenyl, alkynyl, substituted alkynyl, cycloalkyl, substituted cycloalkyl, heterocyclo, substituted heterocyclo, aryl, substituted aryl, cyano, —OR¹³, —C(═O)R¹³, —OC(═O)R¹³, —C(═O)OR¹³, —C(═O)NR¹⁴R¹⁵, —SO₂R¹³, —SO₂OR¹³, —SO₂NR¹⁴R¹⁵, and/or a carbamoyl group which may be substituted by 1 or 2 lower alkyl;
X is —C(═O)—, —C(═S)—, or —SO₂—;
R⁴ is hydrogen, alkyl, substituted alkyl, cyano, —O-lower alkyl, a carbamoyl group which may be substituted by 1 or 2 lower alkyl; a lower alkylene-C(═O)—, or a lower alkylene-O—C(═O)— group;
Y is a bond, lower alkylene, —C(═O)—, or —SO₂—;
R⁵ is alkyl, substituted alkyl, alkenyl, substituted alkenyl, cycloalkyl, substituted cycloalkyl, heterocyclo, substituted heterocyclo, aryl, substituted aryl, —OR⁷, —C(═O)R⁷, —OC(═O)R⁷, —C(═O)OR⁷, or amido which may be substituted by 1 or 2 lower alkyl, aryl, heterocycle or cycloalkyl each of which in turn may optionally be substituted;
alternatively, when m is 1, R⁴ and R⁵ may be linked together to optionally form a five- or six-membered heterocycle;
R⁶ is alkyl, substituted alkyl, alkenyl, substituted alkenyl, alkynyl, substituted alkynyl, halo, cycloalkyl, substituted cycloalkyl, aryl, substituted aryl, heterocyclo, substituted heterocyclo, cyano, nitro, —OC(═O)R⁷, —C(═O)—O—R⁷, —C(═O)R⁷, —C(═S)R⁷, —C(═O)NR⁹R¹⁰, —CR¹¹R¹²OR⁷, —OR⁷, —NR⁹R¹⁰, —SR⁷, —S(═O)R⁷, —SO₂R⁷, —SO₂OR⁷, —SO₂NR⁷R⁸, —P(═O)(OR⁷)(OR⁸), —P(═O)(R⁷)(R⁸), and/or —P(═O)(R⁸)(NHR⁹);
R⁷, R⁸ and R¹³ are each independently hydrogen, alkyl, substituted alkyl, alkenyl, substituted alkenyl, alkynyl, substituted alkynyl, cycloalkyl, substituted cycloalkyl, heterocyclo, substituted heterocyclo, aryl, and/or substituted aryl, except when R⁷ or R¹³is attached to a sulfonyl group as in —S(═O)R⁷, —S(═O)R¹³, —SO₂R⁷, —SO₂R¹³, —SO₂OR⁷, and —SO₂OR¹³, then R⁷ and R¹³ are not hydrogen;
R⁹ and R¹⁰ are each independently hydrogen, alkyl, substituted alkyl, alkenyl, substituted alkenyl, alkynyl, substituted alkynyl, cycloalkyl, substituted cycloalkyl, heterocyclo, substituted heterocyclo, aryl, substituted aryl, —C(═O)R⁷, —C(═O)NHR⁷, —SO₂OR⁷, and/or —SO₂NR⁷R⁸;
R¹¹ and R¹² are each independently hydrogen, alkyl, substituted alkyl, alkenyl, substituted alkenyl, alkynyl, substituted alkynyl, cycloalkyl, substituted cycloalkyl, heterocyclo, substituted heterocyclo, aryl, substituted aryl, halo, cyano, hydroxylamine, hydroxamide, alkoxy, substituted alkoxy, —NR⁷R⁸, thiol, alkylthio, and/or substituted alkylthio;
R¹⁴ and R¹⁵ are each independently hydrogen, alkyl, substituted alkyl, cycloalkyl, substituted cycloalkyl, heterocyclo, substituted heterocyclo, aryl, and/or substituted aryl;
one of a and b is 1, and the other of a and b is 0 or 1;
m is 0 or 1;
p and q are 0, 1, and/or 2; and
r is 0, 1, 2, or 3;
or a pharmaceutically acceptable salt, solvate, or N-oxide thereof.

9. The compound according to claim 8, or a pharmaceutically acceptable salt, solvate, or N-oxide thereof, wherein a and b are both 1.

10. The compound according to claim 9, or a pharmaceutically acceptable salt, solvate, or N-oxide thereof, having the Formula,

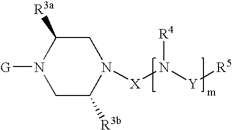

11. The compound according to claim 8, wherein:
X is —C(═O)— or —SO₂—;
R⁴ is hydrogen or lower alkyl;
Y is a bond or methylene;
R⁵ is alkyl, substituted alkyl, phenyl, napthyl, cyclohexyl, O-lower alkylene-phenyl, thienyl, furyl, pyridyl, pyrazinyl, pyrimidinyl, ixosazolyl, thiaziazolyl, benzothiazolyl, benzoimidazolyl, tetrazolyl, morpholinyl, tetrahydrofuryl, thiamorpholinyl, benzofurazanyl, or CO₂(lower alkyl), each of which cyclic groups and alkyl groups of R⁵ may in turn optionally be substituted by one to three groups selected from R¹⁶;
R⁷, R⁸, R⁹, R¹⁰, R¹¹, and R¹² are each independently hydrogen, lower alkyl, or substituted lower alkyl;
R¹⁶ is lower alkyl, halo, nitro, trifluoromethyl, trifluoromethoxy, cyano, alkoxy, phenoxy, alkylthio, hydroxy, carboxy, alkoxycarbonyl, alkylcarbonyloxy, amino, NR¹⁷R¹⁸, carbamoyl, thiol, phenyl, —C(═O)R¹⁷, —C(═O)NR¹⁷R¹⁸, —NR¹⁷SO₂(alkyl), —SO₂NR¹⁷R¹⁸, —S(═O)alkyl, —SO₂alkyl, —O(lower alkylene)-CF₃, phenyl, and/or morpholinyl; and
R¹⁷ and R¹⁸ are independently hydrogen, alkyl, or phenyl;
or a pharmaceutically acceptable salt, solvate, or N-oxide thereof.

12. The compound according to claim 11 having the formula, 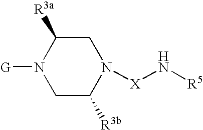
or a pharmaceutically acceptable salt, solvate, or N-oxide thereof.

13. The compound according to claim 8, in which:
G is: 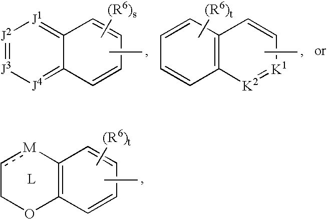
wherein J¹, J², J³ and J⁴ are nitrogen, CH, or CR^{6a}, provided that no more than two of J¹, J^{2,} J³ and J⁴ are nitrogen;
K¹ and K² are nitrogen, CH, or CR^{6a};
M is CH, CR^{6a}, nitrogen, NR^{6a}, or oxygen, wherein when M is oxygen or NR⁶, the bond between M and each adjoining carbon atom is a single bond;
R⁶ is attached to any available carbon atom of the bicyclic group G, and R⁶ and R^{6a} are independently alkyl, substituted alkyl, hydroxy, alkoxy, nitro, cyano, halogen, haloalkyl, haloalkoxy, —OC(═O)(alkyl), —C(═O)—O-(alkyl), —C(═O)H, —C(═O)alkyl, —C(═O)NR⁹R¹⁰, —NR⁹R¹⁰, —SH, —S(alkyl), —S(═O)(alkyl), —SO₂(alkyl), and/or —SO₂NR⁹R¹⁰, wherein R⁹ and R¹⁰ are independently hydrogen, alkyl, cycloalkyl, heterocyclo, and/or aryl, and additionally, R⁶ may be oxo when attached to a saturated or partially-saturated ring L;
s is 0, 1, 2, or 3; and
t is 0, 1, 2, 3, or 4;
or a pharmaceutically acceptable salt, solvate, or N-oxide thereof.

14. The compound according to claim 8, in which:
G is 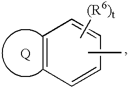
wherein Q is a fused five membered ring selected from one of: 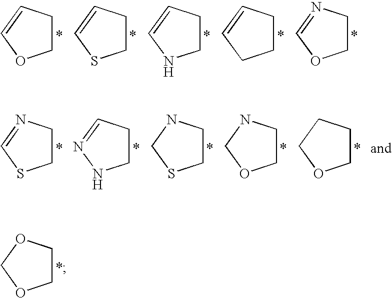
wherein each Q is fused to the phenyl ring along the bond designated with *;
R⁶ is attached to any available carbon atom of the bicyclic group G, including fused ring Q, and is alkyl, substituted alkyl, hydroxy, alkoxy, nitro, cyano, halogen, haloalkyl, haloalkoxy, —OC(═O)(alkyl), —C(═O)—O-(alkyl), —C(═O)H, —C(═O)alkyl, —C(═O)NR⁹R¹⁰, —NR⁹R¹⁰, —SH, —S(alkyl), —S(═O)(alkyl), —SO₂(alkyl), and/or —SO₂NR⁹R¹⁰, wherein R⁹ and R¹⁰ are independently hydrogen, alkyl, cycloalkyl, heterocyclo, and/or aryl, and additionally, where valence allows and R⁶ is attached to ring Q, R⁶ may be oxo; and
t is 0, 1, 2, 3, or 4;
or a pharmaceutically acceptable salt, solvate, or N-oxide thereof.

15. The compound according to claim 8, in which:
G is: 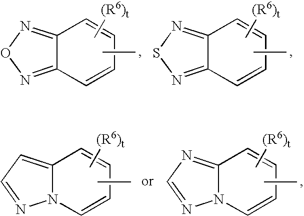
wherein
R6 is attached to any available carbon atom of the bicyclic group G, and is alkyl, substituted alkyl, hydroxy, alkoxy, nitro, cyano, halogen, haloalkyl, haloalkoxy, —OC(═O)(alkyl), —C(═O)—O-(alkyl), —C(═O)H, —C(═O)alkyl, —C(═O)NR⁹R¹⁰, —NR⁹R¹⁰, —SH, —S(alkyl), -S(═O)(alkyl), —SO₂(alkyl), and/or —SO₂—NR⁹R¹⁰, wherein R⁹ and R¹⁰ are independently hydrogen, alkyl, cycloalkyl, heterocyclo, or aryl; and
t is 0, 1, 2, 3, or 4;
or a pharmaceutically acceptable salt, solvate, or N-oxide thereof.

16. The compound according to claim 8, in which:
G is: 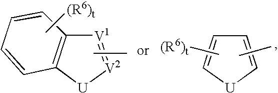
wherein
U is S, O, —S(═O), —S(O)₂, NH, or NR^{6a};
one of V¹ and V² is nitrogen and the other of V¹ and V² is carbon, the group G being attached to the piperazinyl ring via the atom V¹ or V² that is carbon;
R⁶ is attached to any available carbon atom of the group G, and R⁶ and R^{6a} are independently alkyl, hydroxy, alkoxy, nitro, cyano, halogen, haloalkyl, haloalkoxy, —OC(═O)(alkyl), —C(═O)—O-(alkyl), —C(═O)H, —C(═O)alkyl, —C(═O)NR⁹R¹⁰, —NR⁹R¹⁰, —SH, —S(alkyl), —S(═O)(alkyl), —SO₂(alkyl), and/or —SO₂—NR⁹R¹⁰, wherein R⁹ and R¹⁰ are hydrogen, alkyl, cycloalkyl, heterocyclo, and/or aryl; and
t is 0, 1, 2, 3, or 4;
or a pharmaceutically acceptable salt, solvate, or N-oxide thereof.

17. The compound according to claim 8 having the formula, 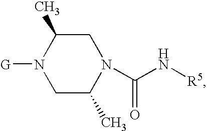
wherein,
R⁵ is an aryl or heteroaryl group optionally substituted with one to three groups selected from R¹⁶;
R¹⁶ is lower alkyl, halo, nitro, trifluoromethyl, trifluoromethoxy, cyano, alkoxy, phenoxy, alkylthio, hydroxy, carboxy, alkoxycarbonyl, alkylcarbonyloxy, amino, NR¹⁷R¹⁸, carbamoyl, thiol, phenyl, —C(═O)R¹⁷, —C(═O)NR¹⁷R¹⁸, —NR¹⁷SO₂(alkyl), —SO₂NR¹⁷R¹⁸, —S(═O)alkyl, —SO₂alkyl, —O(lower alkylene)-CF₃, phenyl, and/or morpholinyl;
R¹⁷ and R¹⁸ are independently hydrogen, alkyl, and/or phenyl; and
G is an optionally substituted bicyclic aryl or heteroaryl;
or a pharmaceutically acceptable salt, solvate, or N-oxide thereof.

18. A pharmaceutical composition comprising an effective amount of a compound of claim 8 and a pharmaceutically acceptable carrier.

19. A method of modulating the function of a nuclear hormone receptor which comprises administering to a mammalian species in need thereof, a pharmaceutically effective amount of a composition according to claim 18.

20. The method of claim 19 wherein said nuclear hormone receptor is a steroid binding nuclear hormone receptor.

21. The method of claim 19 wherein said nuclear hormore receptor is androgen receptor, estrogen receptor, progesterone receptor, glucocorticoid receptor, mineralocorticoid receptor, aldosterone receptor, RORbeta receptor, or COUP-TF2 receptor.

22. A compound having the formula: 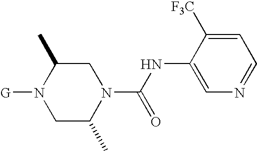
wherein G is: [chemical structure]
